SEQUENCE LISTING (40 sequences)

SEQ ID NO: 1 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 2 - DNA, 936 bp, Homo sapiens
atgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctag

SEQ ID NO: 3 - protein, 402 aa, Homo sapiens
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 4 - DNA, 2226 bp, Homo sapiens
atggggtgtgggctgtcacatggccctgcctaagtaaccacattctcgcttcctccttccacacacagccattgggggttgctcggatccgggactgccgcagggggtgccacagcagtgcctggcagcgtgggctgggaccttgtcactaaagcagagaagccacttcttctgggcccacgaggcagctgtcccatgctctgctgagcacggtggtgccatgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcagtgcctgctggccatcctaatcttggcgctggtggccactatcttcttcgtgtgcactgtggtgctggcggtccgcctctcccgcaagggccacatgtaccccgtgcgtaattactcccccaccgagatggtctgcatctcatccctgttgcctgatgggggtgaggggccctctgccacagccaatgggggcctgtccaaggccaagagcccgggcctgacgccagagcccagggaggaccgtgagggggatgacctcaccctgcacagcttcctcccttagctcactctgccatctgttttggcaagaccccacctccacgggctctcctgggccacccctgagtgcccagaccccattccacagctctgggcttcctcggagacccctggggatggggatcttcagggaaggaactctggccacccaaacaggacaagagcagcctggggccaagcagacgggcaagtggagccacctctttcctccctccgcggatgaagcccagccacatttcagccgaggtccaaggcaggaggccatttacttgagacagattctctcctttttcctgtcccccatcttctctgggtccctctaacatctcccatggctctccccgcttctcctggtcactggagtctcctccccatgtacccaaggaagatggagctcccccatcccacacgcactgcactgccattgtcttttggttgccatggtcaccaaacaggaagtggacattctaagggaggagtactgaagagtgacggacttctgaggctgtttcctgctgctcctctgacttggggcagcttgggtcttcttgggcacctctctgggaaaacccagggtgaggttcagcctgtgagggctgggatgggtttcgtgggcccaagggcagacctttctttgggactgtgtggaccaaggagcttccatctagtgacaagtgacccccagctatcgcctcttgccttcccctgtggccactttccagggtggactctgtcttgttcactgcagtatcccaactgcaggtccagtgcaggcaataaatatgtgatggacaaacgata

SEQ ID NO: 5 - protein, 10 aa, Mus musculus
GFSFTGYTMN

SEQ ID NO: 6 - protein, 8 aa, Mus musculus
GFSFTGYT

SEQ ID NO: 7 - protein, 5 aa, Mus musculus
GYTMN

SEQ ID NO: 8 - protein, 7 aa, Mus musculus
GFSFTGY

SEQ ID NO: 9 - protein, 6 aa, Mus musculus
TGYTMN

SEQ ID NO: 10 - protein, 17 aa, Mus musculus
LISPYNGGTSYNQKFKG

SEQ ID NO: 11 - protein, 8 aa, Mus musculus
ISPYNGGT

SEQ ID NO: 12 - protein, 4 aa, Mus musculus
PYNG

SEQ ID NO: 13 - protein, 13 aa, Mus musculus
WIGLISPYNGGTS

SEQ ID NO: 14 - protein, 10 aa, Mus musculus
LISPYNGGTS

SEQ ID NO: 15 - protein, 9 aa, Mus musculus
RAYGYAMDY

SEQ ID NO: 16 - protein, 11 aa, Mus musculus
ARRAYGYAMDY

SEQ ID NO: 17 - protein, 7 aa, Mus musculus
AYGYAMD

SEQ ID NO: 18 - protein, 10 aa, Mus musculus
ARRAYGYAMD

SEQ ID NO: 19 - protein, 10 aa, Mus musculus
SASSSVSYMY

SEQ ID NO: 20 - protein, 5 aa, Mus musculus
SSVSY

SEQ ID NO: 21 - protein, 6 aa, Mus musculus
SSSVSY

SEQ ID NO: 22 - protein, 6 aa, Mus musculus
SYMYWY

SEQ ID NO: 23 - protein, 7 aa, Mus musculus
DTSNLAS

SEQ ID NO: 24 - protein, 3 aa, Mus musculus
DTS

SEQ ID NO: 25 - protein, 10 aa, Mus musculus
LLIYDTSNLA

SEQ ID NO: 26 - protein, 9 aa, Mus musculus
QQWSSYPFT

SEQ ID NO: 27 - protein, 6 aa, Mus musculus
WSSYPF

SEQ ID NO: 28 - protein, 8 aa, Mus musculus
QQWSSYPF

SEQ ID NO: 29 - protein, 118 aa, Mus musculus
EVQLQQSGPELVKPGASMKISCKASGFSFTGYTMNWVKQSHVKNLEWIGLISPYNGGTSYNQKFKGKATLTVDKSSSTAYMELLSLTSEDSAVYYCARRAYGYAMDYWGQGTSVTVSS

SEQ ID NO: 30 - protein, 106 aa, Mus musculus
QIVLTQSPAIMSASPGEKVTMTCSASSSVSYMYWYQQKPGSSPRLLIYDTSNLASGVPLRFSGSGSGTSYSLTISRMEAEDAATYYCQQWSSYPFTFGSGTKLEIK

SEQ ID NO: 31 - protein, 17 aa, Mus musculus
LISPYDGGTSYNQKFKG

SEQ ID NO: 32 - protein, 8 aa, Mus musculus
ISPYDGGT

SEQ ID NO: 33 - protein, 17 aa, Mus musculus
LISPYDGGTSYNQKFKG

SEQ ID NO: 34 - protein, 13 aa, Mus musculus
WIGLISPYDGGTS

SEQ ID NO: 35 - protein, 10 aa, Mus musculus
LISPYDGGTS

SEQ ID NO: 36 - protein, 118 aa, Mus musculus
EVQLQQSGPELVKPGASMKISCKASGFSFTGYTMNWVKQSHVKNLEWIGLISPYDGGTSYNQKFKGKATLTVDKSSSTAYMELLSLTSEDSAVYYCARRAYGYAMDYWGQGTSVTVSS

SEQ ID NO: 37 - DNA, 1260 bp, Artificial
  VISTA-Fc fusion protein
atgggcgtgcccacagccctggaagctggcagctggaggtggggaagcctgctgttcgccctgtttctggccgcctccctgggacctgtggccgcctttaaggtcgccaccccttacagcctgtacgtgtgccccgagggccagaacgtgaccctgacctgcagactgctgggccctgtggacaagggccacgacgtgaccttctacaagacctggtacaggagcagcaggggcgaggtccagacctgcagcgagaggaggcccatcaggaacctgaccttccaggacctgcacctgcaccacggaggccatcaggccgccaacacctcccacgacctggctcagaggcacggactggagagcgccagcgatcaccacggcaacttcagcatcaccatgaggaacctcaccctgctggacagcggcctgtactgttgcctggtggtggagatcaggcaccaccacagcgagcacagagtgcacggcgccatggaactgcaggtgcagaccggaaaggacgcccccagcaactgcgtggtgtaccccagcagctcccaggacagcgagaacatcaccgccgccagatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 38 - protein, 416 aa, Artificial
  VISTA-Fc fusion protein
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENIRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 521 aa, Artificial
  Fc-fused PSGL-1 with IgG kappa signal sequence and propeptide sequence
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVARSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 523 aa, Artificial
  Fc-fused PSGL-1 with IgG kappa signal sequence and tandem repeat unit
METDTLLLWVLLLWVPGSTGDQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVARSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0243836 A1
(43) Pub. Date: Aug. 3, 2023

(54) RECEPTOR FOR VISTA

(71) Applicant: PIERRE FABRE MEDICAMENT, Boulogne-Billancourt (FR)

(72) Inventors: Pierre FERRÉ, Toulouse (FR); Francisco CRUZALEGUI, Brax (FR); Noureddine LOUKILI, Omex (FR); Olivier DELFOUR, Caraman (FR); Edward Thein Htun VAN DER HORST, Palo Alto, CA (US)

(73) Assignee: PIERRE FABRE MEDICAMENT, Boulogne-Billancourt (FR)

(21) Appl. No.: 17/261,489

(22) PCT Filed: Jul. 22, 2019
(86) PCT No.: PCT/EP2019/069723

§ 371 Date: Nov. 10, 2021

(30) Foreign Application Priority Data
Jul. 20, 2018 (WO) PCT/IB2018/000983

(51) Int. Cl. G01N 33/574; A61P 35/00; C07K 16/28

(52) U.S. Cl. CPC G01N 33/57492; A61P 35/00; G01N 33/57423; G01N 33/5743; C07K 16/2827; C07K 2317/565; C07K 2317/24

(57) ABSTRACT

The present disclosure provides methods for modulating (e.g., preventing, inhibiting, blocking) the interaction of PSGL-1 and VISTA with agents (e.g., antibodies) that bind to PSGL-1 and/or VISTA.

### REFERENCE TO SEQUENCE LISTING

This application includes an electronically submitted sequence listing in .txt format. The .txt file contains a sequence listing entitled “2021-09-17_3493-0752PUS1_ST25.txt” created on Sep. 17, 2021 and is 32,989 bytes in size. The sequence listing contained in this .txt file is part of the specification and is hereby incorporated by reference herein in its entirety.

### INTRODUCTION

Immunotherapy has been a game-changer in the field of cancer therapy. Developments in immune checkpoint-based therapy are progressing at a breathtaking pace. Nevertheless, only a fraction of patients respond to immunotherapies. A particular challenge in cancer immunotherapy has been the identification of mechanism-based biomarkers that could be used to identify candidates for such treatment and guide disease-management decisions (Topalian et al., N Engl J Med, 366(26): 2443-54 (2012)). Therefore, patient selection is an important issue as it will avoid treatment-related toxicity and cost in patients who are unlikely to benefit.

In order to ensure that an immune inflammatory response is not constantly activated once tumor antigens have stimulated a response, multiple controls or “checkpoints” are in place or activated. These checkpoints are mostly represented by T-cell receptor biding to ligands on cells in the surrounding tumor microenvironment, forming immunological synapses which then regulate the function of the T cell.

VISTA (V-Domain Ig Suppressor of T Cell Activation) is a negative checkpoint control protein that regulates T cell activation and immune responses. It is a type I transmembrane protein which contains a single Ig-like V-type domain with homology to similar domains of both the B7 and CD28 families and an intracellular domain. VISTA cytoplasmic tail domain contains two potential protein kinase C binding sites as well as proline residues that could function as docking sites, suggesting that VISTA could potentially function as both a receptor and a ligand.

VISTA is homologous to PDL-1 but displays a unique expression pattern that is restricted to the hematopoietic compartment. VISTA is most highly expressed on myeloid and granulocytic cells, expressed at lower levels on T cells but not present on B cells (Wang et al., JEM 208(3):577-592 (2011); Flies et al., J. Immunology 187(4):1537-1541 (2011)). VISTA is induced on T cells and myeloid cell populations upon activation or immunization, suggesting that inflammation induces its expression (Wang et al., supra). On the other hand, no VISTA expression was detected in tumor cells (Le Mercier et al., Cancer Res; 74:1933-44 (2014)), although it was reported that human gastric cancer cells express VISTA at a low frequency (Böger et al., OncoImmunology, 6:4, e1293215 (2017)). When present, VISTA expression appears restricted to the infiltrating CD11b⁺ cells in the tumor microenvironment of colon or lung cancers. However, it was noted that further studies were required to identify tumor characteristics that may be associated with VISTA expression in the tumor microenvironment (Lines et al., Cancer Immunol Res; 2(6):510-7 (2014)).

VISTA appears to function both as a negative receptor on T cells and as a ligand expressed on APCs interacting with an unknown receptor on T cells.

Several findings suggest that VISTA negatively regulates T cell responses by acting as a ligand that interacts with an unknown receptor on T cells. Like PD-L1, VISTA is a ligand that profoundly suppresses immunity (Lines et al., Cancer Res; 74:1924-32 (2014)), and like PD-L1, blocking VISTA allows for the development of therapeutic immunity to cancer in pre-clinical oncology models (see Le Mercier et al., supra). Whereas blocking VISTA enhances immunity, especially CD8⁺ and CD4⁺ mediated T cell immunity, treatment with a soluble Ig fusion protein of the extracellular domain of VISTA (VISTA-Ig) inhibits T cell proliferation and cytokine production in vitro and overexpression of VISTA on MCA105 tumor cells interferes with the protective antitumor immunity in mice (Wang et al., supra). Moreover, administration of a VISTA-specific monoclonal antibody enhanced CD4⁺ T cell response in vivo and the development of autoimmunity in mice (Wang et al., supra). On the other hand, VISTA appears to have functional activities that are non-redundant with other Ig superfamily members and may play a role in the development of autoimmunity and immune surveillance in cancer. Specifically, although studies using Fc fusion proteins clearly show that VISTA has ligand activity (Wang et al., supra, Lines et al., supra), receptor-like signaling activity has also been described (Flies et al., J Clin Invest; 124:1966-75 (2014)). Indeed, a direct negative role of VISTA as a receptor on T cells is supported by a number of studies.

It is well known that the composition of the immune cell infiltrates varies not only between different tumor entities, but also within tumors of the same anatomic site. Authors have speculated that the response to different immunotherapeutic combinations will probably rely on the patient's immune milieu (Farkona et al., BMC Medicine 14: 73 (2016)). In this regard, PDL-1 expression is known to be induced to evade immune attack (Sharma et al., Cell, 168: 707-23 (2017)). PDL-1 expression shows intratumoral and intertumoral variations (Mino-Kenudson, Cancer Biol Med, 13(2): 157-70 (2016)), but is associated with an objective response to an anti-PD-1 antibody (Topalian et al., supra). On the other hand, the VISTA binding partners that mediate the protein's effects have not been identified yet (Le Mercier et al., Frontiers in Immunology, 6:418 (2015)). Although two phase-I clinical trials with anti-VISTA molecule have been initiated, there is no biomarker capable of predicting a patient's response to these treatments. Thus, there is a need for identifying VISTA's binding partner, as it would facilitate therapeutic development and enable selection of patients susceptible to treatment with anti-VISTA therapeutic agent.

All methods and materials similar or equivalent to those described herein can be used in the practice or testing of the present invention, with suitable methods and materials being described herein. The practice of the invention employs, unless other otherwise indicated, conventional techniques or protein chemistry, molecular virology, microbiology, recombinant DNA technology, and pharmacology, which are within the skill of the art. Such techniques are explained fully in the literature (see e.g., Ausubel et al., Short Protocols in Molecular Biology, Current Protocols; 5th Ed., 2002; Remington's Pharmaceutical Sciences, 17th ed., Mack Publishing Co., Easton, Pa., 1985; and Sambrook et al., Molecular Cloning: A Laboratory Manual, Cold Spring Harbor Laboratory Press; 3rd Ed., 2001). The nomenclatures used in connection with, and the laboratory procedures and techniques of, molecular and cellular biology, protein biochemistry, enzymology and medicinal and pharmaceutical chemistry described herein are those well-known and commonly used in the art. All publications, patent applications, patents, and other references mentioned herein are incorporated by reference in their entirety. Further, the materials, methods, and examples are illustrative only and are not intended to be limiting, unless otherwise specified.

Other features and advantages of the invention will be apparent from the following detailed description, and from the claims.

### FIGURE LEGENDS

FIG. 1 shows a flow chart of the CAPTIREC™ screening procedure using TRICEPS reagents. The Ligand of Interest was a VISTA-Fc fusion protein. The Control ligand was an anti-CD28 antibody.

FIG. 2 shows a Protter illustration of PSGL-1. N-glycosylation sites are represented by residues surrounded by squares and the experimentally observed peptides are represented by filled in circles.

FIG. 3 shows the results of exemplary binding assays for VISTA-Fc to the extracellular domain of PSGL-1 construct A.

FIG. 4 shows the results of exemplary binding assays for VISTA-Fc to the extracellular domain of PSGL-1 construct B.

FIG. 5 shows an exemplary histogram of the detection of PSGL-1 in HL-60 cells by flow cytometry. The isotype and background are represented by the gray shaded peak and the PSGL-1 expressing cells are represented by the white shaded peak.

FIG. 6 shows an exemplary Western Blot detecting the interaction between VISTA and PSGL-1. PSGL-1 is indicated by arrows; incomplete reduction of PSGL-1 is known to result in more than one band.

FIG. 7 shows a bar chart of an anti-VISTA antibody attenuating the interaction between VISTA and PSGL-1. Each bar represents the band intensities corresponding to the PSGL-1 protein. “−” symbol represents no anti-VISTA antibody added. “+” symbol represents pre-incubation with anti-VISTA antibody.

FIG. 8 shows an exemplary histogram of the detection of PSGL-1 in PBMCs by flow cytometry. The isotype and background are represented by the gray shaded peak and the PSGL-1 expressing cells are represented by the white shaded peak.

FIG. 9 shows an exemplary Western Blot showing the co-immunoprecipitation of PSGL-1 using anti-VISTA and anti-PSGL-1 antibodies. PSGL-1 is indicated by an arrow.

FIG. 10 shows PSGL-1 expression in exemplary flow cytometry assays of nave Et resting, effector and exhausted effector T cell subsets.

FIG. 11 shows PSGL-1 expression in exemplary flow cytometry assays of circulating central memory and circulating effector memory T cell subsets.

FIG. 12 shows an example of multiplex staining of mRNA for PSGL1, VISTA and PDL1 on a squamous lung tumor

FIG. 13 shows a bar chart of PSGL-1 inhibiting VISTA-dependent IL-2 release from CD4⁺ T cells.

### DEFINITIONS

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The term “about” or “approximately” refers to the normal range of error for a given value or range known to the person of skills in the art. It usually means within 20%, such as within 10%, or within 5% (or 1% or less) of a given value or range.

As used herein, “administer” or “administration” refers to the act of injecting or otherwise physically delivering a substance as it exists outside the body (e.g., an anti-PSGL-1 antibody and/or anti-VISTA antibody provided herein) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

As used herein, an “antagonist” or “inhibitor” refers to a molecule that is capable of inhibiting or otherwise decreasing one or more of the biological activities of a target protein, such as PSGL-1, VISTA or a different co-inhibitory molecule described herein. In some embodiments, an antagonist of PSGL-1 (e.g., an antagonistic antibody provided herein) can, for example, act by inhibiting or otherwise decreasing the activation and/or cell signaling pathways of the cell expressing PSGL-1 (e.g., a T cell) and/or the cell expressing VISTA (e.g., a VISTA-bearing tumor cell, a regulatory T cell, a myeloid-derived suppressor cell or a suppressive dendritic cell), thereby inhibiting a biological activity of the cell relative to the biological activity in the absence of the antagonist. In some embodiments the antibodies provided herein are antagonistic anti-PSGL-1 antibodies. In some embodiments, an antagonist of a co-inhibitory molecule (e.g., an antagonistic antibody against VISTA, CD86, CD80, PDL-1, PDL-2, CTLA-4, PD1, LAG3, BTNL2, B7-H3, B7-H4, a butyrophilin, CD48, CD244, TIM-3, CD200R, CD200, CD160, BTLA, HVEM, LAIR1, TIM1, Galectin 9, TIM3, CD48, 2B4, CD155, CD112, CD113 or TIGIT) can, for example, act by inhibiting or otherwise decreasing the activation and/or cell signaling pathways of the cell expressing the co-inhibitory molecule (e.g., a T cell or an antigen-presenting cell), thereby inhibiting a biological activity of the cell relative to the biological activity in the absence of the antagonist. In some embodiment, the antagonist molecule is an antagonistic antibody, i.e. an antibody that inhibits or reduces one or more of the biological activities of an antigen, such as PSGL-1, VISTA or a different co-inhibitory molecule described herein. Certain antagonistic antibodies substantially or completely inhibit one or more of the biological activities of said antigen.

As used herein, an “agonist” or “activator” refers to a molecule that is capable of activating or otherwise increasing one or more of the biological activities of a target protein, such as a co-stimulatory molecule. In some embodiments, an agonist of a co-stimulatory molecule (e.g., an agonistic antibody of CD154, TNFRSF25, GITR, 4-1BB, OX40, CD27, TMIGD2, ICOS, CD28, CD40, TL1A, GITRL, 41BBL, OX40L, CD70, HHLA2, ICOSL, a cytokine, LIGHT, HVEM, CD30, CD30L, B7-H2, CD80, CD86, CD40L, TIM4, TIM1, SLAM, CD48, CD58, CD155, CD112, DR3, GITR, CD2, and CD226) may, for example, act by activating or otherwise increasing the activation and/or cell signaling pathways of the cell expressing the co-stimulatory molecule (e.g., a T cell or an antigen-presenting cell), thereby increasing a biological activity of the cell relative to the biological activity in the absence of the agonist. In some embodiment, the agonist molecule is an agonistic antibody, i.e. an antibody that activates or increases one or more of the biological activities of an antigen, such as PSGL-1, VISTA or a different co-inhibitory molecule described herein. Certain agonistic antibodies substantially or completely activate one or more of the biological activities of said antigen.

The terms “antibody” and “immunoglobulin” or “Ig” are used interchangeably herein. These terms are used herein in the broadest sense and specifically cover monoclonal antibodies (including full length monoclonal antibodies) of any isotype such as IgG, IgM, IgA, IgD, and IgE, polyclonal antibodies, multispecific antibodies, chimeric antibodies, and antibody fragments, provided that said fragments retain the desired biological function. An antibody reactive with a specific antigen can be generated by recombinant methods such as selection of libraries of recombinant antibodies in phage or similar vectors, or by immunizing an animal with the antigen or an antigen-encoding nucleic acid. These terms are intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxy-terminal portion of each chain includes a constant region (See, Borrebaeck (ed.) (1995) Antibody Engineering, Second Ed., Oxford University Press.; Kuby (1997) Immunology, Third Ed., W. H. Freeman and Company, New York). In some embodiments, the specific molecular antigen can be bound by an antibody provided herein includes the target PSGL-1 polypeptide, fragment or epitope.

Antibodies also include, but are not limited to, synthetic antibodies, monoclonal antibodies, recombinantly produced antibodies, multispecific antibodies (including bi-specific antibodies), human antibodies, humanized antibodies, camelized antibodies, chimeric antibodies, intrabodies, anti-idiotypic (anti-Id) antibodies, and functional fragments of any of the above, which refers a portion of an antibody heavy or light chain polypeptide that retains some or all of the binding activity of the antibody from which the fragment was derived. Non-limiting examples of functional fragments include single-chain Fvs (scFv) (e.g., including monospecific, bispecific, etc.), Fab fragments, F(ab′) fragments, F(ab)₂ fragments, F(ab′)₂ fragments, disulfide-linked Fvs (sdFv), Fd fragments, Fv fragments, diabody, triabody, tetrabody and minibody. In particular, antibodies provided herein include immunoglobulin molecules and immunologically active portions of immunoglobulin molecules, e.g., antigen binding domains or molecules that contain an antigen-binding site that binds to a VISTA antigen (e.g., one or more complementarity determining regions (CDRs) of an anti-VISTA antibody). Such antibody fragments can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Plückthun and Skerra, Meth. Enzymol., 178:497-515 (1989) and in Day, E. D., Advanced Immunochemistry, Second Ed., Wiley-Liss, Inc., New York, N.Y. (1990). The antibodies provided herein can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule. Anti-PSGL-1 antibodies or anti-VISTA antibodies provided herein can be agonistic antibodies or antagonistic antibodies.

The terms “anti-PSGL-1 antibodies,” “antibodies that bind to PSGL-1,” “antibodies that bind to a PSGL-1 epitope,” and analogous terms are used interchangeably herein and refer to antibodies that bind to a PSGL-1 polypeptide, such as a PSGL-1 antigen or epitope. Such antibodies include humanized antibodies. An antibody that binds to a PSGL-1 antigen may be cross-reactive with related antigens. In some embodiments, an antibody that binds to PSGL-1 does not cross-react with other antigens. In some embodiments, an anti-PSGL-1 antibody described herein does not block or inhibit the binding of PSGL-1 to P-selectin, L-selectin or E-selectin. An antibody that binds to PSGL-1 can be identified, for example, by immunoassays, BIAcore, or other techniques known to those of skill in the art. An antibody binds to PSGL-1, for example, when it binds to PSGL-1 with higher affinity than to any cross-reactive antigen as determined using experimental techniques, such as radioimmunoassays (RIA) and enzyme-linked immunosorbent assays (ELISAs), for example, an antibody that specifically binds to PSGL-1. Typically, a specific or selective reaction will be at least twice background signal or noise and may be more than 10 times background. See, e.g., Paul, ed., 1989, Fundamental Immunology Second Edition, Raven Press, New York at pages 332-336 for a discussion regarding antibody specificity. In some embodiments, an antibody “which binds” an antigen of interest is one that binds the antigen with sufficient affinity such that the antibody is useful as a diagnostic and/or therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein as determined by fluorescence activated cell sorting (FACS) analysis or radioimmunoprecipitation (RIA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{D} for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In some embodiments, the term “specific binding” refers to binding where a molecule binds to a particular polypeptide or epitope on a particular polypeptide without substantially binding to any other polypeptide or polypeptide epitope. In some embodiments, an antibody that binds to PSGL-1 or VISTA has a dissociation constant (K_{D}) of ≤1 μM, ≤100 nM, ≤10 nM, ≤1 nM, or ≤0.1 nM. In some embodiments, anti-PSGL-1 antibody or anti-VISTA antibody binds to an epitope of PSGL-1 or VISTA that is conserved among PSGL-1 or VISTA from different species.

An “antigen” is a predetermined antigen to which an antibody can selectively bind. The target antigen may be a polypeptide, carbohydrate, nucleic acid, lipid, hapten or other naturally occurring or synthetic compound. In some embodiments, the target antigen is a polypeptide, including, for example, a PSGL-1 polypeptide.

The term “antigen binding fragment,” “antigen binding domain,” “antigen binding region,” and similar terms refer to that portion of an antibody which comprises the amino acid residues that interact with an antigen and confer on the binding agent its specificity and affinity for the antigen (e.g., the complementarity determining regions (CDRs)).

The term “antigen-presenting cell” or “APC” refers to a heterogeneous group of immune cells that mediate the cellular immune response by processing and presenting antigens for recognition by certain lymphocytes, such as T cells. APCs include, but are not limited to, dendritic cells, macrophages, Langerhans cells and B cells.

The terms “binds” or “binding” as used herein refer to an interaction between molecules to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions or forces. The strength of the total non-covalent interactions between a single antigen-binding site on an antibody and a single epitope of a target molecule, such as PSGL-1, is the affinity of the antibody or functional fragment for that epitope. The ratio of association (k₁) to dissociation (k₋₁) of an antibody to a monovalent antigen (k₁/k₋₁) is the association constant K, which is a measure of affinity. The value of K varies for different complexes of antibody and antigen and depends on both k₁ and k₋₁. The association constant K for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent PSGL-1, come in contact with antibodies containing multiple binding sites, the interaction of antibody with antigen at one site will increase the probability of a reaction at a second site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The term “biological sample” refers to a sample that has been obtained from a biological source, such as a patient or subject. In some embodiments, a biological sample includes, but is not limited to, whole blood, partially purified blood, PBMCs, tissue biopsies, and the like. Preferably, a biological sample is a tumor sample. In some preferred embodiments, a biological sample is obtained by a tissue biopsy (e.g., tumor biopsy, which can include immune infiltrates).

The term “block,” or a grammatical equivalent thereof, when used in the context of an antibody refers to an antibody that prevents or stops a biological activity of the antigen to which the antibody binds. A blocking antibody includes an antibody that combines with an antigen without eliciting a reaction, but that blocks another protein from later combining or complexing with that antigen. The blocking effect of an antibody can be one which results in a measurable change in the antigen's biological activity. In some embodiments, an anti-PSGL-1 antibody described herein blocks the ability of VISTA to bind PSGL-1, which can result in inhibiting or blocking suppressive signals of VISTA. Certain anti-PSGL-1 antibodies described herein inhibit or block suppressive signals of VISTA on VISTA-expressing cells, including by about 98% to about 100% as compared to the appropriate control (e.g., the control being cells not treated with the antibody being tested). In some embodiments, the anti-PSGL-1 antibody described herein blocks the binding of PSGL-1 to the extracellular domain VISTA and/or blocks the binding of a VISTA-expressing cell to a PSGL-1-expressing cell. In some embodiments, the anti-PSGL-1 antibody described herein does not block the binding of PSGL-1 to a protein other than VISTA, such as P-selectin, L-selectin, and/or E-selectin.

The term “VISTA” or “VISTA polypeptide” and similar terms refers to the polypeptide (“polypeptide,” “peptide” and “protein” are used interchangeably herein) encoded by the human Chromosome 10 Open Reading Frame 54 (VISTA) gene, which is also known in the art as B7-H5, platelet receptor Gi24, GI24, Stress Induced Secreted Protein1, SISP1, and PP2135, for example, comprising the amino acid sequence of:

(SEQ ID NO: 1)

| | |
|---|---|
| 1 | mgvptaleag swrwgsllfa iflaaslgpv |
| | aafkvatpys lyvcpegqnv tltcrllgpv |
| 61 | dkghdvtfyk twyrssrgev qtcserrpir |
| | nltfqdlhlh hgghqaants hdlaqrhgle |
| 121 | sasdhhgnfs itmrnltlld sglycclvve |
| | irhhhsehrv hgamelqvqt gkdapsncvv |
| 181 | ypsssqdsen itaaalatga civgilclpl |
| | illlvykqrq aasnrraqel vrmdsniqgi |
| 241 | enpgfeaspp aqgipeakvr hplsyvaqrq |
| | psesgrhlls epstplsppg pgdvffpsld |
| 301 | pvpdspnfev i |

and related polypeptides, including SNP variants thereof. The VISTA polypeptide has been shown to or is predicted to comprise several distinct regions within the amino acid sequence including: a signal sequence (residues 1-32; see Zhang et al., Protein Sci. 13:2819-2824 (2004)); an immunoglobulin domain—IgV-like (residues 33-162); and a transmembrane region (residues 195-215). The mature VISTA protein includes amino acid residues 33-311 of SEQ ID NO: 1. The extracellular domain of the VISTA protein includes amino acid residues 33-194 of SEQ ID NO: 1. Related polypeptides include allelic variants (e.g., SNP variants); splice variants; fragments; derivatives; substitution, deletion, and insertion variants; fusion polypeptides; and interspecies homologs, preferably, which retain VISTA activity and/or are sufficient to generate an anti-VISTA immune response. VISTA can exist in a native or denatured form. The VISTA polypeptides described herein may be isolated from a variety of sources, such as from human tissue types or from another source, or prepared by recombinant or synthetic methods. A “native sequence VISTA polypeptide” comprises a polypeptide having the same amino acid sequence as the corresponding VISTA polypeptide derived from nature. Such native sequence VISTA polypeptides can be isolated from nature or can be produced by recombinant or synthetic means. The term “native sequence VISTA polypeptide” specifically encompasses naturally-occurring truncated or secreted forms of the specific VISTA polypeptide (e.g., an extracellular domain sequence), naturally-occurring variant forms (e.g., alternatively spliced forms) and naturally-occurring allelic variants of the polypeptide.

A cDNA nucleic acid sequence encoding the VISTA polypeptide, for example, comprises:

(SEQ ID NO: 2)

| | |
|---|---|
| 1 | atgggcgtcc ccacggccct ggaggccggc |
| | agctggcgct ggggatccct gctcttcgct |
| 61 | ctcttcctgg ctgcgtccct aggtccggtg |
| | gcagccttca aggtcgccac gccgtattcc |
| 121 | ctgtatgtct gtcccgaggg gcagaacgtc |
| | accctcacct gcaggctctt gggccctgtg |
| 181 | gacaaagggc acgatgtgac cttctacaag |
| | acgtggtacc gcagctcgag gggcgaggtg |
| 241 | cagacctgct cagagcgccg gcccatccgc |
| | aacctcacgt tccaggacct tcacctgcac |
| 301 | catggaggcc accaggctgc caacaccagc |
| | cacgacctgg ctcagcgcca cgggctggag |
| 361 | tcggcctccg accaccatgg caacttctcc |
| | atcaccatgc gcaacctgac cctgctggat |
| 421 | agcggcctct actgctgcct ggtggtggag |
| | atcaggcacc accactcgga gcacagggtc |
| 481 | catggtgcca tggagctgca ggtgcagaca |
| | ggcaaagatg caccatccaa ctgtgtggtg |
| 541 | tacccatcct cctcccagga tagtgaaaac |
| | atcacggctg cagccctggc tacgggtgcc |
| 601 | tgcatcgtag gaatcctctg cctccccctc |
| | atcctgctcc tggtctacaa gcaaaggcag |
| 661 | gcagcctcca accgccgtgc ccaggagctg |
| | gtgeggatgg acagcaacat tcaagggatt |
| 721 | gaaaaccccg gctttgaagc ctcaccacct |
| | gcccagggga tacccgaggc caaagtcagg |
| 781 | caccccctgt cctatgtggc ccagcggcag |
| | ccttctgagt ctgggcggca tctgctttcg |
| 841 | gagcccagca cccccctgtc tcctccaggc |
| | cccggagacg tcttcttccc atccctggac |
| 901 | cctgtccctg actctccaaa ctttgaggtc |
| | atctag |

As described herein, VISTA is an immunomodulator, that is a negative checkpoint regulator of immune responses (e.g., inhibits or suppresses immune responses). As also described herein, PSGL-1 is a receptor of VISTA. As also described herein, methods for modulating (e.g., preventing, inhibiting, blocking) the interaction of PSGL-1 and VISTA with agents (e.g., antibodies) that bind to PSGL-1 and/or VISTA, are useful, including, for example, for inhibiting or blocking suppressive signals of VISTA. Modulating the interaction of VISTA and PSGL-1 can result in an increased immune response, including an increase in immune activation (e.g., T cell activation such as T cell proliferation). Antibodies that bind to VISTA, useful in methods as described herein, include those disclosed in WO2014/197849 (PCT/US2014/041388).

Orthologs to the VISTA polypeptide are also well known in the art. For example, the mouse ortholog to the VISTA polypeptide is V-region Immunoglobulin-containing Suppressor of T cell Activation (VISTA) (also known as PD-L3, PD-1H, PD-XL, Pro1412 and UNQ730), which shares approximately 70% sequence identity to the human polypeptide. Orthologs of VISTA can also be found in additional organisms including chimpanzee, cow, rat and zebrafish.

A “VISTA-expressing cell,” “a cell having expression of VISTA” or a grammatical equivalent thereof refers to a cell that expresses endogenous or transfected VISTA on the cell surface. VISTA expressing cells include VISTA-bearing tumor cells, regulatory T cells (e.g., CD4⁺ Foxp3⁺ regulatory T cells), myeloid-derived suppressor cells (e.g., CD11b⁺ or CD11b^{high} myeloid-derived suppressor cells) and/or suppressive dendritic cells (e.g., CD11b⁺ or CD11b^{high} dendritic cells). A cell expressing VISTA produces sufficient levels of VISTA on its surface, such that an anti-VISTA antibody can bind thereto and/or PSGL-1 or a cell expressing PSGL-1 can bind thereto. In some aspects, inhibition or blocking of such binding may have a therapeutic effect. A cell that “overexpresses” VISTA is one that has significantly higher levels of VISTA at the cell surface thereof, compared to a cell of the same tissue type that is known to express VISTA. Such overexpression may be caused by gene amplification or by increased transcription or translation. VISTA overexpression may be determined in a diagnostic or prognostic assay by evaluating increased levels of the VISTA protein present on the surface of a cell (e.g. via an immunohistochemistry assay; FACS analysis). Alternatively, or additionally, one may measure levels of VISTA-encoding nucleic acid or mRNA in the cell, e.g. via fluorescent in situ hybridization; (FISH; see WO98/45479 published October, 1998), Southern blotting, Northern blotting, or polymerase chain reaction (PCR) techniques, such as real time quantitative PCR (RT-PCR). Aside from the above assays, various in vivo assays are available to the skilled practitioner. For example, one may expose cells within the body of the patient to an antibody which is optionally labeled with a detectable agent, and binding of the antibody to cells in the patient can be evaluated, e.g. by external scanning for radioactivity or by analyzing a biopsy taken from a patient previously exposed to the antibody. A VISTA-expressing tumor cell includes, but is not limited to, acute myeloid leukemia (AML) tumor cells.

A “VISTA-mediated disease,” “VISTA-mediated disorder” and “VISTA-mediated condition” are used interchangeably and refer to any disease, disorder or condition that is completely or partially caused by or is the result of VISTA. Such diseases, disorders or conditions include those caused by or otherwise associated with VISTA, including by or associated with VISTA-expressing cells (e.g., tumor cells, myeloid-derived suppressor cells (MDSC), suppressive dendritic cells (suppressive DC), and/or regulatory T cells (T-regs)). In some embodiments, VISTA is aberrantly (e.g., highly) expressed on the surface of a cell. In some embodiments, VISTA may be aberrantly upregulated on a particular cell type. In other embodiments, normal, aberrant or excessive cell signaling is caused by binding of VISTA to a VISTA receptor (e.g., PSGL-1), which can bind or otherwise interact with VISTA.

The terms “cell proliferative disorder” and “proliferative disorder” refer to disorders that are associated with some degree of abnormal cell proliferation. In some embodiments, the cell proliferative disorder is a tumor or cancer. “Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, oral cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain cancer, as well as head and neck cancer, and associated metastases. In some embodiments, the cancer is a hematological cancer, which refers to cancer that begins in blood-forming tissue, such as the bone marrow, or in the cells of the immune system. Examples of a hematologic cancer are leukemia (e.g., acute myeloid leukemia (AML), acute lymphoblastic leukemia (ALL), chronic myelogenous leukemia (CML), chronic lymphocytic leukemia (CLL), or acute monocytic leukemia (AMoL)), lymphoma (Hodgkin lymphoma or non-Hodgkin lymphoma), and myeloma (multiple myeloma, plasmacytoma, localized myeloma or extramedullary myeloma).

A “co-inhibitory molecule” (also known as a “negative checkpoint regulator” or “NCR”) refers to a molecule that down-regulates immune responses (e.g., T cell activation) by delivery of a negative signal to T cells following their engagement by ligands or counter-receptors. Exemplary functions of a co-inhibitory molecule is to prevent out-of-proportion immune activation, minimize collateral damage, and/or maintain peripheral self-tolerance. In some embodiments, a co-inhibitory molecule is a ligand or receptor expressed by an antigen presenting cell. In some embodiments, a co-inhibitory molecule is a ligand or receptor expressed by a T cell. In some embodiments, a co-inhibitory molecule is a ligand or receptor expressed by both an antigen presenting cell and a T cell.

A “co-stimulatory molecule” refers to a molecule that up-regulates immune responses (e.g., T cell activation) by delivery of a positive signal to T cells following their engagement by ligands or counter-receptors. For a T cell to become fully activated, two signals are required: 1) an antigen-specific signal is provided through the T cell receptor interacting with peptide-MHC molecules on an antigen presenting cell; and 2) a co-stimulatory signal, which is antigen nonspecific, and is provided by the interaction between co-stimulatory molecules expressed on the membrane of an antigen presenting cell and the T cell. T cell co-stimulation provides for T cell proliferation, differentiation and survival. In some embodiments, a co-stimulatory molecule is a ligand or receptor expressed by an antigen presenting cell. In some embodiments, a co-stimulatory molecule is a ligand or receptor expressed by a T cell. In some embodiments, a co-stimulatory molecule is a ligand or receptor expressed by both an antigen presenting cell and a T cell.

A “chemotherapeutic agent” is a chemical or biological agent (e.g., an agent, including a small molecule drug or biologic, such as an antibody or cell) useful in the treatment of cancer, regardless of mechanism of action. Chemotherapeutic agents include compounds used in targeted therapy and conventional chemotherapy. Examples of chemotherapeutic agents include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, AR1NOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammal I and calicheamicin omega II (see, e.g., Agnew, Chem Intl. Ed. Engl. 33:183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN®, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. Additional chemotherapeutic agents include cytotoxic agents useful as antibody drug conjugates, such as maytansinoids (DM1 and DM4, for example) and auristatins (MMAE and MMAF, for example).

Also included in the definition of chemotherapeutic agent are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY 117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RI VISor® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as ME inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAX1D®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech) (x) immunomodulatory agents such as Bispecific T Cell Engager (BITE) antibodies and chimeric antigen receptor (CAR) T cells; and pharmaceutically acceptable salts, acids and derivatives of any of the above.

A “CDR” refers to one of three hypervariable regions (H1, H2 or H3) within the non-framework region of the immunoglobulin (Ig or antibody) VH ß-sheet framework, or one of three hypervariable regions (L1, L2 or L3) within the non-framework region of the antibody VL ß-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved ß-sheet framework, and thus are able to adapt different conformations (Chothia and Lesk, J. Mol. Biol. 196:901-917 (1987)). Both terminologies are well recognized in the art. CDR region sequences have also been defined by AbM, Contact and IMGT. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

The term “hypervariable region”, “HVR”, or “HV”, when used herein refers to the regions of an antibody variable domain that are hypervariable in sequence and/or form structurally defined loops. Generally, antibodies comprise six hypervariable regions; three in the VH (H1, H2, H3), and three in the VL (LI, L2, L3). A number of hypervariable region delineations are in use and are encompassed herein. The Kabat CDRs are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J Mol. Bioi. 196:901-917 (1987)). The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structuRes. The residues from each of these hypervariable regions are noted below.

Recently, a universal numbering system has been developed and widely adopted, ImMunoGeneTics (IMGT) Information System® (Lafranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. Correspondence between the Kabat numbering and the IMGT unique numbering system is also well known to one skilled in the art (e.g. Lefranc et al., supra). An Exemplary system, shown herein, combines Kabat and Chothia.

TABLE 1
CDR Definitions

| | Exemplary | | | | | |
|---|---|---|---|---|---|---|
| | (Kabat + | | | | | |
| | Chothia) | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (LI), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. The variable domain residues are 25 numbered according to Kabat et al., supra, for each of these definitions. As used herein, the terms “HVR” and “CDR” are used interchangeably.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CL domain of the light chain.

In the context of a polypeptide, the term “derivative” as used herein refers to a polypeptide that comprises an amino acid sequence of a PSGL-1 polypeptide, a fragment of a PSGL-1 polypeptide, or an antibody that binds to a PSGL-1 polypeptide which has been altered by the introduction of amino acid residue substitutions, deletions or additions. The term “derivative” as used herein also refers to a PSGL-1 polypeptide, a fragment of a PSGL-1 polypeptide, or an antibody that binds to a PSGL-1 polypeptide which has been chemically modified, e.g., by the covalent attachment of any type of molecule to the polypeptide. For example, but not by way of limitation, a PSGL-1 polypeptide, a fragment of a PSGL-1 polypeptide, or a PSGL-1 antibody may be chemically modified, e.g., by glycosylation, acetylation, pegylation, phosphorylation, amidation, derivatization by known protecting/blocking groups, proteolytic cleavage, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. A derivative of a PSGL-1 polypeptide, a fragment of a PSGL-1 polypeptide, or a PSGL-1 antibody may be chemically modified by chemical modifications using techniques known to those of skill in the art, including, but not limited to specific chemical cleavage, acetylation, formulation, metabolic synthesis of tunicamycin, etc. Further, a derivative of a PSGL-1 polypeptide, a fragment of a PSGL-1 polypeptide, or a PSGL-1 antibody may contain one or more non-classical amino acids. A polypeptide derivative possesses a similar or identical function as a PSGL-1 polypeptide, a fragment of a PSGL-1 polypeptide, or a PSGL-1 antibody described herein.

The term “detectable probe,” as used herein, refers to a composition that provides a detectable signal. The term includes, without limitation, any fluorophore, chromophore, radiolabel, enzyme, antibody or antibody fragment, and the like, that provide a detectable signal via its activity.

The term “diagnostic agent” refers to a substance administered to a subject that aids in the diagnosis of a disease. Such substances can be used to reveal, pinpoint, and/or define the localization of a disease-causing process. In some embodiments, a diagnostic agent includes a substance that is conjugated to an antibody provided herein, that when administered to a subject or contacted to a sample from a subject, aids in the diagnosis of cancer, tumor formation, or any other VISTA-mediated disease, disorder or condition.

The term “detectable agent” refers to a substance that can be used to ascertain the existence or presence of a desired molecule, such as an antibody provided herein, in a sample or subject. A detectable agent can be a substance that is capable of being visualized or a substance that is otherwise able to be determined and/or measured (e.g., by quantitation).

The term “detecting” as used herein encompasses quantitative or qualitative detection.

The term “encode” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

The term “epitope” as used herein refers to the region of an antigen, such as PSGL-1 polypeptide or PSGL-1 polypeptide fragment, to which an antibody binds. Preferably, an epitope as used herein is a localized region on the surface of an antigen, such as PSGL-1 polypeptide or PSGL-1 polypeptide fragment, that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, for example, by an immunoassay. Antigenic epitopes need not necessarily be immunogenic. Epitopes usually consist of chemically active surface groupings of molecules such as amino acids or sugar side chains and have specific three-dimensional structural characteristics as well as specific charge characteristics. An epitope can be formed by contiguous residues or by non-contiguous residues brought into close proximity by the folding of an antigenic protein. Epitopes formed by contiguous amino acids are typically retained on exposure to denaturing solvents, whereas epitopes formed by non-contiguous amino acids are typically lost under said exposure. In some embodiments, a PSGL-1 epitope is a three-dimensional surface feature of a PSGL-1 polypeptide. In other embodiments, a PSGL-1 epitope is linear feature of a PSGL-1 polypeptide. Generally, an antigen has several or many different epitopes and reacts with many different antibodies.

The term “excipient” as used herein refers to an inert substance which is commonly used as a diluent, vehicle, preservative, binder, or stabilizing agent, and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, Remington's Pharmaceutical Sciences (1990) Mack Publishing Co., Easton, Pa., which is hereby incorporated by reference in its entirety.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In some embodiments, PSGL-1 or VISTA fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a PSGL-1 or VISTA polypeptide or an antibody that binds to a PSGL-1 or VISTA polypeptide. In some embodiments, a fragment of a PSGL-1 or VISTA polypeptide or an antibody that binds to a PSGL-1 or VISTA antigen retains at least 1, at least 2, or at least 3 functions of the polypeptide or antibody.

The term “framework” or “FR” residues refers to those variable domain residues other than the hypervariable region residues herein defined. FR residues are those variable domain residues flanking the CDRs. FR residues are present, e.g., in chimeric, humanized, human, domain antibodies, diabodies, linear antibodies, and bispecific antibodies.

A “functional fragment” of an antibody will exhibit at least one if not some or all of the biological functions attributed to the intact antibody, the function comprising at least specific binding to the target antigen.

The term “fusion protein” as used herein refers to a polypeptide that comprises an amino acid sequence of an antibody and an amino acid sequence of a heterologous polypeptide or protein (e.g., a polypeptide or protein not normally a part of the antibody (e.g., a non-anti-PSGL-1 antibody or a non-anti-VISTA antibody)). The term “fusion” when used in relation to PSGL-1, VISTA, an anti-PSGL-1 antibody, or an anti-VISTA antibody refers to the joining of a peptide or polypeptide, or fragment, variant and/or derivative thereof, with a heterologous peptide or polypeptide. In some embodiments, the fusion protein retains the biological activity of the PSGL-1, the VISTA, the anti-PSGL-1 antibody, or the anti-VISTA antibody. In some embodiments, the fusion protein comprises an anti-PSGL-1 antibody or an anti-VISTA antibody VH domain, VL domain, VH CDR (one, two or three VH CDRs), and/or VL CDR (one, two or three VL CDRs), wherein the fusion protein binds to a PSGL-1 or VISTA epitope.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “hinge region” refers herein to a flexible amino acid stretch in the central part of the heavy chains of the IgG and IgA immunoglobulin classes, which links these 2 chains by disulfide bonds. The hinge region is generally defined as stretching from Glu216 to Pro230 of human IgG1 (Burton, Mol Immunol, 22: 161-206, 1985). Hinge regions of other IgG isotypes may be aligned with the IgG1 sequence by placing the first and last cysteine residues forming inter-heavy chain S—S bonds in the same positions. The “CH2 domain” of a human IgG Fc portion (also referred to as “Cγ2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain (Burton, Mol Immunol, 22: 161-206, 1985). The “CH3 domain” comprises the stretch of residues C-terminal to a CH2 domain in an Fc portion (i.e., from about amino acid residue 341 to about amino acid residue 447 of an IgG).

The term “host” as used herein refers to an animal, such as a mammal (e.g., a human).

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

“Humanized” forms of nonhuman (e.g., murine) antibodies are chimeric antibodies that include human immunoglobulins (recipient antibody) in which the native CDR residues are replaced by residues from the corresponding CDR of a nonhuman species (donor antibody) such as mouse, rat, rabbit or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable domains, in which all or substantially all of the CDRs correspond to those of a nonhuman immunoglobulin and all or substantially all of the FRs are those of a human immunoglobulin sequence. In some embodiments, the humanized antibody will comprise at least a portion of an immunoglobulin constant region (Fc), typically that of a human immunoglobulin. For further details, see, Jones et al., Nature, 321:522-525 (1986); Riechmann et al., Nature, 332:323-329 (1988); and Presta, Curr. Op. Struct. Biol., 2:593-596 (1992); Carter et al., Proc. Natl. Acad. Sci. USA 89:4285-4289 (1992); and U.S. Pat. No. 6,800,738 (issued Oct. 5, 2004), U.S. Pat. No. 6,719,971 (issued Sep. 27, 2005), U.S. Pat. No. 6,639,055 (issued Oct. 28, 2003), U.S. Pat. No. 6,407,213 (issued Jun. 18, 2002), and U.S. Pat. No. 6,054,297 (issued Apr. 25, 2000).

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the agent, the route of administration, etc. In some embodiments, effective amount also refers to the amount of an antibody provided herein to achieve a specified result (e.g., inhibition of a PSGL-1 or VISTA biological activity of a cell, such as modulating T cell activation). In some embodiments, this term refers to the amount of a therapy (e.g., an antibody provided herein) which is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy (e.g., a therapy other than anti-PSGL-1 antibody provided herein). In some embodiments, the effective amount of an antibody is from about 0.1 mg/kg (mg of antibody per kg weight of the subject) to about 100 mg/kg. In some embodiments, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein).

The term “inhibit,” or a grammatical equivalent thereof, when used in the context of an antibody refers to an antibody that suppresses, restrains or decreases a biological activity of the antigen to which the antibody binds. The inhibitory effect of an antibody can be one which results in a measurable change in the antigen's biological activity. In some embodiments, an anti-PSGL-1 antibody described herein inhibits the ability of VISTA to bind PSGL-1, which can result in inhibiting the co-inhibitory activity of VISTA. Certain anti-PSGL-1 antibodies described herein inhibit or block suppressive signals of VISTA on VISTA-expressing cells by greater than 5%, such as from about 5% to about 50%, or by greater than 50% (e.g., from about 50% to about 98%) as compared to the appropriate control (e.g., the control being cells not treated with the antibody being tested). In some embodiments, the anti-PSGL-1 antibody described herein inhibit the binding of PSGL-1 to the extracellular domain VISTA and/or inhibit the binding of a VISTA-expressing cell to a PSGL-1-expressing cell. Additionally, in some embodiments, the anti-PSGL-1 antibody described herein does not inhibit the binding of PSGL-1 to a protein other than VISTA, such as P-selectin, L-selectin, and/or E-selectin.

The term “immune infiltrate” or “tumor immune cells” refers to cells that infiltrate the microenvironment of a tumor, including, but not limited to, lymphocytes (e.g., T cells, B-cells, natural killer (NK) cells), dendritic cells, mast cells, and macrophages.

As used herein, the term “in combination” in the context of the administration of other therapies refers to the use of more than one therapy (e.g., an anti-PSGL-1 antibody and an anti-VISTA antibody). The use of the term “in combination” does not restrict the order or the time in which therapies are administered to a subject (e.g., one therapy before, concurrent with, or after another therapy). A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a VISTA-mediated disease, disorder or condition. Any additional therapy can be administered in any order or time with the other additional therapies (e.g., an anti-PSGL-1 antibody and an anti-VISTA antibody). In some embodiments, the antibodies can be administered in combination with one or more therapies (e.g., therapies that are not the antibodies that are currently administered to prevent, treat, manage, and/or ameliorate a VISTA-mediated disease, disorder or condition). Non-limiting examples of therapies that can be administered in combination with an antibody include an antagonist to a co-inhibitory molecule, an agonist to a co-stimulatory molecule, a chemotherapeutic agent, radiation, analgesic agents, anesthetic agents, antibiotics, or immunomodulatory agents or any other agent listed in the U.S. Pharmacopoeia and/or Physician's Desk Reference.

An “isolated” antibody is substantially free of cellular material or other contaminating proteins from the cell or tissue source and/or other contaminant components from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). In some embodiments, when the antibody is recombinantly produced, it is substantially free of culture medium, e.g., culture medium represents less than about 20%, 10%, or 5% of the volume of the protein preparation. In some embodiments, when the antibody is produced by chemical synthesis, it is substantially free of chemical precursors or other chemicals, e.g., it is separated from chemical precursors or other chemicals which are involved in the synthesis of the protein. Accordingly, such preparations of the antibody have less than about 30%, 20%, 10%, 5% (by dry weight) of chemical precursors or compounds other than the antibody of interest. Contaminant components can also include, but are not limited to, materials that would interfere with therapeutic uses for the antibody, and may include enzymes, hormones, and other proteinaceous or nonproteinaceous solutes. In some embodiments, the antibody will be purified (1) to greater than 95% by weight of antibody as determined by the Lowry method (Lowry et al. J. Bio. Chem. 193: 265-275, 1951), such as 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step. In some embodiments, antibodies provided herein are isolated.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In some embodiments, a nucleic acid molecule(s) encoding an antibody provided herein is isolated or purified.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

As used herein, the terms “manage,” “managing,” and “management” refer to the beneficial effects that a subject derives from a therapy (e.g., a prophylactic or therapeutic agent), which does not result in a cure of the disease. In some embodiments, a subject is administered one or more therapies (e.g., prophylactic or therapeutic agents, such as an antibody provided herein) to “manage” a VISTA-mediated disease, disorder or condition, including one or more symptoms thereof, so as to prevent the progression or worsening of the disease, disorder or condition.

The term “monoclonal antibody” refers to an antibody obtained from a population of homogenous or substantially homogeneous antibodies, i.e. the antibodies forming this population are essentially identical except for possible naturally occurring mutations which might be present in minor amounts. In other words, a monoclonal antibody is a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is generally characterized by heavy chains of one and only one class and subclass, and light chains of only one type. These antibodies are highly specific and are directed against a single antigen. In addition, in contrast with preparations of polyclonal antibodies which typically include various antibodies directed against various determinants, or epitopes, each monoclonal antibody is directed against a single epitope of the antigen. In some embodiments, a “monoclonal antibody,” as used herein, is an antibody produced by a single hybridoma or other cell, wherein the antibody binds to only a VISTA epitope as determined, e.g., by ELISA or other antigen-binding or competitive binding assay known in the art. The term “monoclonal” is not limited to any particular method for making the antibody. For example, monoclonal antibodies provided herein may be made by the hybridoma method as described in Kohler et al.; Nature, 256:495 (1975) or may be isolated from phage libraries using the techniques. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Other exemplary methods of producing other monoclonal antibodies are provided in the Examples herein.

The term “native” when used in connection with biological materials such as nucleic acid molecules, polypeptides, host cells, and the like, refers to those which are found in nature and not manipulated by a human being.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

“Polyclonal antibodies” as used herein refers to an antibody population generated in an immunogenic response to a protein having many epitopes and thus includes a variety of different antibodies directed to the same and to different epitopes within the protein. Methods for producing polyclonal antibodies are known in the art (See, e.g., see, for example, Chapter 11 in: Short Protocols in Molecular Biology, (2002) 5th Ed., Ausubel et al., eds., John Wiley and Sons, New York).

As used herein, the term “polynucleotide,” “nucleotide,” nucleic acid” “nucleic acid molecule” and other similar terms are used interchangeable and include DNA, RNA, mRNA and the like.

As used herein, the terms “prevent,” “preventing,” and “prevention” refer to the total or partial inhibition of the development, recurrence, onset or spread of a VISTA-mediated disease, disorder or condition and/or symptom related thereto, resulting from the administration of a therapy or combination of therapies provided herein (e.g., a combination of prophylactic or therapeutic agents, such as an antibody provided herein).

As used herein, the term “prophylactic agent” refers to any agent that can totally or partially inhibit the development, recurrence, onset or spread of a VISTA-mediated disease, disorder or condition, and/or symptom related thereto in a subject. In some embodiments, the term “prophylactic agent” refers to an anti-PSGL-1 antibody provided herein. In some other embodiments, the term “prophylactic agent” refers to an agent other than an anti-PSGL-1 antibody provided herein. In some embodiments, a prophylactic agent is an agent which is known to be useful to or has been or is currently being used to prevent a VISTA-mediated disease, disorder or condition, and/or a symptom related thereto or impede the onset, development, progression and/or severity of a VISTA-mediated disease, disorder or condition, and/or a symptom related thereto. In some embodiments, the prophylactic agent is a humanized anti-PSGL-1 antibody, such as a humanized anti-PSGL-1 monoclonal antibody.

The term “P-selectin glycoprotein ligand 1” (also known as PSGL-1, PSGL1, selectin P ligand, SELPLG, CLA, and CD162,) refers to a polypeptide (“polypeptide,” “peptide” and “protein” are used interchangeably herein) encoded by the SELPLG gene, for example, comprising the amino acid sequence:

```
(SEQ ID NO: 3)
    1 mplqllllli llgpgnslql wdtwadeaek
        algpllardr rqateyeyld ydfipetepp
  61 emlrnstdtt pltgpgtpes ttvepaarrs
        tgldaggavt elttelanmg nlstdsaame
121 iqttqpaate aqttplaate aqttrltate
        aqttplaate aqttppaate aqttqptgle
181 aqttapaame aqttapaame aqttppaame
        aqttqttame aqttapeate aqttqptate
241 aqttplaame alstepsate alsmepttkr
        glfipfsvss vthkgipmaa snisvnypvg
301 apdhisvkqc llaililalv atiffvctvv
        lavrlsrkgh mypvrnyspt emvcissllp
361 dggegpsata nggls kaksp gltpepredr
        egdditlhs f lp
```

and related polypeptides, including SNP variants thereof. PSLG-1 is a human mucin-type glycoprotein ligand that is known to bind all three selectins (P-selectin, E-selectin, and L-selectin), but it binds P-selectin with the highest affinity (McEver et al., J. Clin. Invest., 100(3):485-492 (1997) and Carlow et al., Immunological Reviews, 230:75-96 (2009)). PSGL-1 is a disulfide-bonded homodimer with two 120-kD subunits and is expressed on the surface of monocytes, lymphocytes, granulocytes, and in some CD34⁺ stem cells. As such, this protein has been known to play a role in leukocyte trafficking during inflammation by tethering of leukocytes to activated platelets or endothelia expressing selectins. PSGL-1 typically has two post-translational modifications, tyrosine sulfation and the addition of the sialyl Lewis x tetrasaccharide (sLex) to its O-linked glycans, for its high-affinity binding activity. Aberrant expression of the SELPLG gene and polymorphisms in this gene are associated with defects in innate and adaptive immune responses.

As those skilled in the art will appreciate, an anti-PSGL-1 antibody provided herein can bind to a PSGL-1 polypeptide, polypeptide fragment, antigen, and/or epitope, as an epitope is part of the larger antigen, for example, which is part of the larger polypeptide fragment, which, in turn, for example, is part of the larger polypeptide. PSGL-1 can exist in a native or denatured form. The PSGL-1 polypeptides described herein may be isolated from a variety of sources, such as from human tissue types or from another source, or prepared by recombinant or synthetic methods. A “native sequence PSGL-1 polypeptide” comprises a polypeptide having the same amino acid sequence as the corresponding PSGL-1 polypeptide derived from nature. Such native sequence PSGL-1 polypeptides can be isolated from nature or can be produced by recombinant or synthetic means. The term “native sequence PSGL-1 polypeptide” specifically encompasses naturally-occurring truncated or secreted forms of the specific PSGL-1 polypeptide (e.g., an extracellular domain sequence), naturally-occurring variant forms (e.g., alternatively spliced forms) and naturally-occurring allelic variants of the polypeptide.

A cDNA nucleic acid sequence encoding a PSGL-1 polypeptide, for example, comprises:

```
(SEQ ID NO: 4)
      1 atggggtgtg ggctgtcaca tggccctgcc taagtaacca cattctcgct tcctccttcc
    61 acacacagcc attgggggtt gctcggatcc gggactgccg cagggggtgc cacagcagtg
  121 cctggcagcg tgggctggga ccttgtcact aaagcagaga agccacttct tctgggccca
  181 cgaggcagct gtcccatgct ctgctgagca cggtggtgcc atgcctctgc aactcctcct
  241 gttgctgatc ctactgggcc ctggcaacag cttgcagctg tgggacacct gggcagatga
  301 agccgagaaa gccttgggtc ccctgcttgc ccgggaccgg agacaggcca ccgaatatga
  361 gtacctagat tatgatttcc tgccagaaac ggagcctcca gaaatgctga ggaacagcac
  421 tgacaccact cctctgactg ggcctggaac ccctgagtct accactgtgg agcctgctgc
  481 aaggcgttct actggcctgg atgcaggagg ggcagtcaca gagctgacca cggagctggc
  541 caacatgggg aacctgtcca cggattcagc agctatggag atacagacca ctcaaccagc
  601 agccacggag gcacagacca ctccactggc agccacagag gcacagacaa ctcgactgac
  661 ggccacggag gcacagacca ctccactggc agccacagag gcacagacca ctccaccagc
  721 agccacggaa gcacagacca ctcaacccac aggcctggag gcacagacca ctgcaccagc
  781 agccatggag gcacagacca ctgcaccagc agccatggaa gcacagacca ctccaccagc
  841 agccatggag gcacagacca ctcaaaccac agccatggag gcacagacca ctgcaccaga
  901 agccacggag gcacagacca ctcaacccac agccacggag gcacagacca ctccactggc
  961 agccatggag gccctgtcca cagaacccag tgccacagag gccctgtcca tggaacctac
1021 taccaaaaga ggtctgttca tacccttttc tgtgtcctct gttactcaca agggcattcc
1081 catggcagcc agcaatttgt ccgtcaacta cccagtgggg gccccagacc acatctctgt
1141 gaagcagtgc ctgctggcca tcctaatctt ggcgctggtg gccactatct tcttcgtgtg
1201 cactgtggtg ctggcggtcc gcctctcccg caagggccac atgtaccccg tgcgtaatta
1261 ctcccccacc gagatggtct gcatctcatc cctgttgcct gatgggggtg aggggccctc
1321 tgccacagcc aatgggggcc tgtccaaggc caagagcccg ggcctgacgc cagagcccag
1381 ggaggaccgt gagggggatg acctcaccct gcacagcttc ctcccttagc tcactctgcc
1441 atctgttttg gcaagacccc acctccacgg gctctcctgg gccacccctg agtgcccaga
1501 ccccattcca cagctctggg cttcctcgga gacccctggg gatggggatc ttcagggaag
1561 gaactctggc cacccaaaca ggacaagagc agcctggggc caagcagacg ggcaagtgga
1621 gccacctctt tcctccctcc gcggatgaag cccagccaca tttcagccga ggtccaaggc
1681 aggaggccat ttacttgaga cagattctct cctttttcct gtcccccatc ttctctgggt
1741 ccctctaaca tctcccatgg ctctccccgc ttctcctggt cactggagtc tcctccccat
1801 gtacccaagg aagatggagc tcccccatcc cacacgcact gcactgccat tgtcttttgg
1861 ttgccatggt caccaaacag gaagtggaca ttctaaggga ggagtactga agagtgacgg
1921 acttctgagg ctgtttcctg ctgctcctct gacttggggc agcttgggtc ttcttgggca
1981 cctctctggg aaaacccagg gtgaggttca gcctgtgagg gctgggatgg gtttcgtggg
2041 cccaagggca gacctttctt tgggactgtg tggaccaagg agcttccatc tagtgacaag
2101 tgacccccag ctatcgcctc ttgccttccc ctgtggccac tttccagggt ggactctgtc
2161 ttgttcactg cagtatccca actgcaggtc cagtgcaggc aataaatatg tgatggacaa
2221 aegata
```

Orthologs to the human PSGL-1 polypeptide are also well known in the art. For example, orthologs of PSGL-1 can be found in organisms such as mouse (Mus musculus), rat (Rattus norvegicus), dog (Canis lupus familiaris), cattle (Bos Taurus), zebrafish (Danio rerio), horse (Equus caballus), chimpanzee (Pan troglodytes), etc.

A “PSGL-1-mediated disease,” “PSGL-1-mediated disorder” and “PSGL-1-mediated condition” are used interchangeably and refer to any disease, disorder or condition that is completely or partially caused by or is the result of PSGL-1. Such diseases, disorders or conditions include those caused by or otherwise associated with PSGL-1, including by or associated with PSGL-1-expressing cells (e.g., tumor cells, myeloid-derived suppressor cells (MDSC), suppressive dendritic cells (suppressive DC), and/or regulatory T cells (T-regs)). In some embodiments, PSGL-1 is aberrantly (e.g., highly) expressed on the surface of a cell. In some embodiments, PSGL-1 may be aberrantly upregulated on a particular cell type. In other embodiments, normal, aberrant or excessive cell signaling is caused by binding of PSGL-1 to a PSGL-1 ligand (e.g., VISTA), which can bind or otherwise interact with PSGL-1. In preferred embodiment, the PSGL-1-mediated disease is caused by binding of PSGL-1 to a specific PSGL-1 ligand (e.g., VISTA) but not to the other ligands (e.g., the selectins).

The term “radiation,” when used in the therapeutic context refers to a type of treatment that uses a beam of intense energy to kill target cells (e.g., cancer cells). Radiation therapy includes the use of X-rays, protons or other forms of energy that are administered through an external beam. Radiation therapy also includes radiation treatment that is placed into a patient's body (e.g., brachytherapy) whereby a small container of radioactive material is implanted directly into or near a tumor.

The terms “relative expression level” refers to a quantification of the expression level of a protein in a given sample relative to another reference protein in the same sample and/or to another reference sample. In the context of the methods described herein, the level of expression of PSGL-1 can be expressed in absolute numbers, such as based on a standard curve, or can be expressed in relative expression levels against one or more other proteins that are assayed in the sample (e.g., VISTA, CD11b, CD33, CD4, or CD8).

The term “recombinant antibody” refers to an antibody that is prepared, expressed, created or isolated by recombinant means. Recombinant antibodies can be antibodies expressed using a recombinant expression vector transfected into a host cell, antibodies isolated from a recombinant, combinatorial antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of immunoglobulin gene sequences to other DNA sequences. Such recombinant antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In some embodiments, however, such recombinant antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, the term “side effects” encompasses unwanted and adverse effects of a therapy (e.g., a prophylactic or therapeutic agent). Unwanted effects are not necessarily adverse. An adverse effect from a therapy (e.g., a prophylactic or therapeutic agent) might be harmful or uncomfortable or risky. Examples of side effects include, diarrhea, cough, gastroenteritis, wheezing, nausea, vomiting, anorexia, abdominal cramping, fever, pain, loss of body weight, dehydration, alopecia, dyspenea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physician's Desk Reference (67^{th} ed., 2013).

As used herein, the terms “subject” and “patient” are used interchangeably. As used herein, in some embodiments, a subject is a mammal, such as a non-primate (e.g., cows, pigs, horses, cats, dogs, rats, etc.) ora primate (e.g., monkey and human). In some embodiments, the subject is a human. In some embodiments, the subject is a mammal (e.g., a human) having a VISTA-mediated disease, disorder or condition and/or a symptom related thereto. In another embodiment, the subject is a mammal (e.g., a human) at risk of developing a VISTA-mediated disease, disorder or condition and/or a symptom related thereto.

As used herein “substantially all” refers to refers to at least about 60%, at least about 70%, at least about 75%, at least about 80%, at least about 85%, at least about 90%, at least about 95%, at least about 98%, at least about 99%, or about 100%.

As used herein, the term “therapeutic agent” refers to any agent that can be used in treating, preventing or alleviating a disease, disorder or condition, including in the treatment, prevention or alleviation of one or more symptoms of a VISTA-mediated disease, disorder, or condition and/or a symptom related thereto. In some embodiments, a therapeutic agent refers to an anti-PSGL-1 antibody provided herein. In some embodiments, a therapeutic agent refers to an agent other than an anti-PSGL-1 antibody provided herein. In some embodiments, a therapeutic agent is an agent which is known to be useful for, or has been or is currently being used for the treatment, prevention or alleviation of one or more symptoms of a VISTA-mediated disease, disorder, condition, and/or a symptom related thereto.

The combination of therapies (e.g., use of therapeutic agents) can be more effective than the additive effects of any two or more single therapies. For example, a synergistic effect of a combination of therapeutic agents permits the use of lower dosages of one or more of the agents and/or less frequent administration of the agents to a subject with a VISTA-mediated disease, disorder or condition and/or a symptom related thereto. The ability to utilize lower dosages of therapeutic therapies and/or to administer the therapies less frequently reduces the toxicity associated with the administration of the therapies to a subject without reducing the efficacy of the therapies in the prevention, treatment or alleviation of one or more symptoms of a VISTA-mediated disease, disorder or condition and/or a symptom related thereto. In addition, a synergistic effect can result in improved efficacy of therapies in the prevention, treatment or alleviation of one or more symptoms of a VISTA-mediated disease, disorder or condition and/or a symptom related thereto. Finally, synergistic effect of a combination of therapies (e.g., therapeutic agents) may avoid or reduce adverse or unwanted side effects associated with the use of any single therapy.

The term “therapeutically effective amount” as used herein refers to the amount of a therapeutic agent (e.g., an anti-PSGL antibody or any other therapeutic agent, including as described herein, including, for example, an anti-VISTA antibody) that is sufficient to reduce and/or ameliorate the severity and/or duration of a given disease, disorder or condition and/or a symptom related thereto. A therapeutically effective amount of a therapeutic agent can be an amount necessary for the reduction or amelioration of the advancement or progression of a given disease, disorder or condition, reduction or amelioration of the recurrence, development or onset of a given disease, disorder or condition and/or to improve or enhance the prophylactic or therapeutic effect of another therapy (e.g., a therapy other than the administration of an anti-PSGL-1 antibody, including as described herein).

As used herein, the term “therapy” refers to any protocol, method and/or agent that can be used in the prevention, management, treatment and/or amelioration of a VISTA-mediated disease, disorder or condition. In some embodiments, the terms “therapies” and “therapy” refer to a biological therapy, supportive therapy, and/or other therapies useful in the treatment, prevention and/or amelioration of a VISTA-mediated disease, disorder or condition known to one of skill in the art such as medical personnel.

As used herein, the terms “treat,” “treatment” and “treating” refer to the reduction or amelioration of the progression, severity, and/or duration of a VISTA-mediated disease, disorder or condition, resulting from the administration of one or more therapies (including, but not limited to, the administration of one or more therapeutic agents, such as an anti-PSGL-1 antibody, including as described herein). In some embodiments, such terms refer to the reduction or inhibition of cancer (e.g., a hematological cancer). In some embodiments, such terms refer to the reduction or amelioration of the progression, severity, and/or duration of a disease, disorder or condition, that is responsive to immune modulation, such modulation resulting from increasing T cell activation.

The term “tumor microenvironment” refers to the cellular environment in which a tumor exists. A tumor microenvironment can include surrounding blood vessels, immune cells, fibroblasts, bone marrow-derived inflammatory cells, lymphocytes, signaling molecules and the extracellular matrix.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains differ extensively in sequence between different antibodies. The variability in sequence is concentrated in the CDRs while the less variable portions in the variable domain are referred to as framework regions (FR). Each variable region comprises three CDRs which are connected to four FR. The CDRs of the light and heavy chains are primarily responsible for the interaction of the antibody with antigen. Although not directly involved in antigen binding, the FR determines the folding of the molecules and thus the amount of CDR that is presented on the surface of the variable region for interaction with the antigen. In some embodiments, the variable region is a human variable region.

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g. residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Unless stated otherwise herein, references to residue numbers in the variable domain of antibodies means residue numbering by the Kabat numbering system. Other numbering systems have been described, including, for example, by AbM, Chothia, Contact and IMGT.

The term “variant” when used in relation to PSGL-1, VISTA or to an anti-PSGL-1 antibody or an anti-VISTA antibody refers to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a PSGL-1 or VISTA variant may result from one or more (such as, for example, about 1 to about 25, about 1 to about 20, about 1 to about 15, about 1 to about 10, or about 1 to about 5) changes to an amino acid sequence of native PSGL-1 or VISTA, respectively. Also by way of example, a variant of an anti-PSGL-1 antibody or anti-VISTA antibody may result from one or more (such as, for example, about 1 to about 25, about 1 to about 20, about 1 to about 15, about 1 to about 10, or about 1 to about 5) changes to an amino acid sequence of a native or previously unmodified anti-PSGL-1 antibody or anti-VISTA antibody. Variants may be naturally occurring, such as allelic or splice variants, or may be artificially constructed. Polypeptide variants may be prepared from the corresponding nucleic acid molecules encoding the variants. In some embodiments, the PSGL-1 variant, VISTA variant or anti-PSGL-1 antibody or an anti-VISTA antibody variant at least retains PSGL-1, VISTA, anti-PSGL-1 antibody or anti-VISTA antibody functional activity, respectively. In some embodiments, an anti-PSGL-1 antibody variant binds PSGL-1 and/or is antagonistic to PSGL-1 activity. In some embodiments, an anti-VISTA antibody variant binds VISTA and/or is antagonistic to VISTA activity. In some embodiments, the variant is encoded by a single nucleotide polymorphism (SNP) variant of a nucleic acid molecule that encodes PSGL-1, VISTA, anti-PSGL-1 antibody or anti-VISTA antibody VH or VL regions or subregions.

The term “vector” refers to a substance that is used to introduce a nucleic acid molecule into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g. both an antibody heavy and light chain), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecule is expressed in a sufficient amount to produce the desired product (e.g. an anti-PSGL-1 antibody provided herein), and it is further understood that expression levels can be optimized to obtain sufficient expression using methods well known in the art.

### DETAILED DESCRIPTION

The practice of the disclosure employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, N.Y.; Ausubel et al., Current Protocols in Molecular Biology, John Wiley Et Sons (1987 and annual updates); Current Protocols in Immunology, John Wiley Et Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

### PSGL-1 Isa Receptor for Vista

During carcinogenesis, tumor cells interact with a complex microenvironment which is composed of extracellular matrix and non-neoplastic host cells, including mesenchymal cells, vascular endothelial cells and inflammatory or immune cells. The microenvironment plays a crucial role in suppressing tumor-specific T-cell responses. In order to ensure that an immune inflammatory response is not constantly activated once tumor antigens have stimulated a response, multiple checkpoints are in place or activated. These checkpoints are mostly represented by T-cell receptor binding to ligands on cells in the surrounding microenvironment, forming immunological synapses which then regulate the functions of the T cell.

VISTA is one these immune checkpoints. The protein is hematopoietically restricted and in multiple cancer models, it was only detected on tumor infiltrating leukocytes and not on tumor cells. VISTA negatively regulates T cell immunity via direct impact on T cells by engaging different receptor/ligand, as, unique among immune checkpoint proteins, it acts both as a ligand and a receptor (Le Mercier, supra).

The present inventors have now identified PSGL-1 as a binding partner (e.g., a ligand or a receptor) of the VISTA protein. PSGL-1 is a homodimeric 120-kDa transmembrane glycoprotein bearing O- and N-linked glycans whose best-known role is in immune cell trafficking via selectin binding. PSGL-1 is expressed in cells of lymphoid, myeloid cells, and dendritic lineages (Laszik et al., Blood, 88(8): 3010-21 (1996)). Naive T cells express the non-selectin-binding form of PSGL-1, which can engage potentially other currently unknown binding partners (Veerman et al., Nat. Immunol. 8(5), 532-539 (2007)). Expression in tumor cells has also been observed. It has been recently demonstrated that PSGL-1 promotes T cell exhaustion, thus facilitating melanoma tumor growth, through the induction of an unknown partner (Tinoco et al., Immunity, 44: 1190-03 (2016)).

The inventors have demonstrated direct binding between the two proteins and shown that PSGL-1 and VISTA cooperate in preventing T cell activation. Indeed, the physical interaction between VISTA and PSGL-1 underlies a functional one, as both genes are co-expressed in a number of tumors. None of the other putative VISTA receptors display such co-localization, emphasizing the specificity of the relationship. Moreover, VISTA and PSGL-1 are expressed in tumor cell microenvironment. More specifically, in situ hybridization revealed that both genes are expressed in within adjacent cells in tumor microenvironment. Every PSGL-1 expressing cell is adjacent to a VISTA-expressing cell in immune infiltrates, indicating that PSGL-1 is a reliable proxy for activated VISTA.

### Diagnosis of Vista-Mediated Disorders

The above data indicate that PSGL-1 is dependable biomarker for diagnosing a VISTA-mediated disorder, such as a VISTA-mediated cancer. Reagents such as labeled nucleic acid probes or antibodies provided herein, which bind to PSGL-1 nucleic acid or protein can thus be used for diagnostic purposes to detect, diagnose, or monitor a VISTA-mediated disease, disorder or condition.

Thus, in a first aspect, the invention relates to an in vitro method for detecting a VISTA-mediated cancer in a subject, said method comprising the steps of:
a) contacting a biological sample of said subject with a reagent capable of binding PSGL-1 protein or nucleic acid; and
b) detecting the binding of said reagent with said biological sample.

According to the present method, the binding of PSGL-1 indicates the presence of VISTA-mediated cancer. Preferably, the binding of PSGL-1 in immune infiltrates of the tumor microenvironment indicates the presence of VISTA-mediated cancer.

The reagent capable of binding PSGL-1 protein or nucleic acid may be any reagent or compound known to the person of skills in the art which is capable of binding specifically to PSGL-1. For example, the skilled person will immediately realize that a DNA or RNA probe which hybridizes specifically with PSGL-1 binds specifically to PSGL-1. Likewise, the skilled person will immediately realize that an anti-PSGL-1 antibody such as those described herein binds specifically to PSGL-1.

The invention also relates to an in vitro method for detecting a VISTA-mediated cancer in a subject, said method comprising the steps of:
a) contacting a biological sample of said subject with a reagent capable of binding PSGL-1 protein or nucleic acid; and
b) quantifying the binding of said reagent with said biological sample.

According to the present method, the binding of PSGL-1 indicates the presence of VISTA-mediated cancer. Preferably, the binding of PSGL-1 in immune infiltrates of the tumor microenvironment indicates the presence of VISTA-mediated cancer.

As will be apparent to the skilled artisan, the level of reagent binding to PSGL-1 may be quantified by any means known to the person of skills in the art, as detailed hereafter. Preferred methods include the use of immunoenzymatic assays, such as ELISA or ELISPOT, immunofluorescence, immunohistochemistry (IHC), radio-immunoassay (RIA), or FACS.

The quantification of step b) of the present method is a direct reflection of the level of PSGL-1 expression in the sample, notably in immune infiltrates of the tumor microenvironment. The present method thus allows for identifying a VISTA-mediated cancer by determining the level of expression of PSGL-1, as described above. In a preferred embodiment, the level of expression of PSGL-1 in said sample, notably in immune infiltrates of the tumor microenvironment, is compared to a reference level.

According to a further preferred embodiment, the invention relates to an in vitro method for detecting a VISTA-mediated cancer in a subject, said method comprising the steps of:
a) determining the level of expression of PSGL-1 in a biological sample of said subject; and
b) comparing the level of expression of step a) with a reference level;
   wherein an increase in the assayed level of PSGL-1 in step a) compared to the reference level is indicative of a VISTA-mediated disease, disorder or condition.

The invention also relates to an in vitro method for diagnosing a VISTA-mediated cancer in a subject, said method comprising the steps of:
a) determining the level of expression of PSGL-1 in a biological sample of said subject; and
b) comparing the level of expression of step a) with a reference level;
   wherein an increase in the assayed level of PSGL-1 in step (b) compared to the reference level is indicative of a VISTA-mediated disease, disorder or condition.

The expression level of PSGL-1 is advantageously compared or measured in relation to levels in a control cell or sample also referred to as a “reference level” or “reference expression level”. “Reference level”, “reference expression level”, “control level” and “control” are used interchangeably in the specification. A “control level” means a separate baseline level measured in a comparable control cell, which is generally disease or cancer free. The said control cell may be from the same individual, since, even in a cancerous patient, the tissue which is the site of the tumor still comprises non-tumor healthy tissue. It may also originate from another individual who is normal or does not present with the same disease from which the diseased or test sample is obtained. Within the context of the present invention, the term “reference level” refers to a “control level” of expression of PSGL-1 used to evaluate a test level of expression of PSGL-1 in a cancer cell-containing sample of a patient. For example, when the level of PSGL-1 in the biological sample of a patient is higher than the reference level of PSGL-1, the cells will be considered to have a high level of expression, or overexpression, of PSGL-1. The reference level can be determined by a plurality of methods. Expression levels may thus define PSGL-1 bearing cells or alternatively the level of expression of PSGL-1 independent of the number of cells expressing PSGL-1. Thus, the reference level for each patient can be prescribed by a reference ratio of PSGL-1, wherein the reference ratio can be determined by any of the methods for determining the reference levels described herein.

For example, the control may be a predetermined value, which can take a variety of forms. It can be a single cut-off value, such as a median or mean. The “reference level” can be a single number, equally applicable to every patient individually, or the reference level can vary, according to specific subpopulations of patients. Thus, for example, older men might have a different reference level than younger men for the same cancer, and women might have a different reference level than men for the same cancer. Alternatively, the “reference level” can be determined by measuring the level of expression of PSGL-1 in non-oncogenic cancer cells from the same tissue as the tissue of the neoplastic cells to be tested. As well, the “reference level” might be a certain ratio of PSGL-1 in the neoplastic cells of a patient relative to the PSGL-1 levels in non-tumor cells within the same patient. The “reference level” can also be a level of PSGL-1 of in vitro cultured cells, which can be manipulated to simulate tumor cells, or can be manipulated in any other manner which yields expression levels which accurately determine the reference level. On the other hand, the “reference level” can be established based upon comparative groups, such as in groups not having elevated PSGL-1 levels and groups having elevated PSGL-1 levels. Another example of comparative groups would be groups having a particular disease, condition or symptoms and groups without the disease. The predetermined value can be arranged, for example, where a tested population is divided equally (or unequally) into groups, such as a low-risk group, a medium-risk group and a high-risk group.

The reference level can also be determined by comparison of the level of PSGL-1 in populations of patients having the same cancer. This can be accomplished, for example, by histogram analysis, in which an entire cohort of patients is graphically presented, wherein a first axis represents the level of PSGL-1, and a second axis represents the number of patients in the cohort whose tumor cells express PSGL-1 at a given level. Two or more separate groups of patients can be determined by identification of subsets populations of the cohort which have the same or similar levels of PSGL-1. Determination of the reference level can then be made based on a level which best distinguishes these separate groups. A reference level also can represent the levels of two or more markers, one of which is PSGL-1. Two or more markers can be represented, for example, by a ratio of values for levels of each marker.

Likewise, an apparently healthy population will have a different ‘normal’ range than will have a population which is known to have a condition associated with expression of PSGL-1. Accordingly, the predetermined value selected may take into account the category in which an individual falls. Appropriate ranges and categories can be selected with no more than routine experimentation by those of ordinary skill in the art. By “elevated” “increased” it is meant high relative to a selected control. Typically, the control will be based on apparently healthy normal individuals in an appropriate age bracket.

It will also be understood that the controls according to the invention may be, in addition to predetermined values, samples of materials tested in parallel with the experimental materials. Examples include tissue or cells obtained at the same time from the same subject, for example, parts of a single biopsy, or parts of a single cell sample from the subject.

Preferably, the reference level of PSGL-1 is the level of expression of PSGL-1 in normal tissue samples (e.g., from a patient not having a VISTA-mediated disease, disorder or condition, or from the same patient before disease onset).

In some embodiments, expression of a given protein is an indication of the presence of a certain type of cell in a sample. For example, the expression of PSGL-1, CD4 and/or CD8 by cells in the sample can indicate the presence of T cells in the sample. Likewise, expression of VISTA alone or in combination with CD11 b or CD33 by cells in the sample can indicate the presence of VISTA-bearing tumor cells, regulatory T cells (e.g., CD4⁺ Foxp3⁺ regulatory T cells), myeloid-derived suppressor cells (e.g., CD11b⁺ or CD11b^{high} and/or CD33⁺ myeloid-derived suppressor cells) and/or suppressive dendritic cells (e.g., CD11 b⁺ or CD11b^{high} dendritic cells). Preferably, expression of VISTA, CD11b, CD33, CD4, and CD8, notably in immune infiltrates of the tumor microenvironment, indicates the presence of a VISTA-mediated cancer in a subject.

According to these specific embodiment, the in vitro method for detecting a VISTA-mediated cancer in a subject comprises the steps of:
a) determining the level of expression of PSGL-1 and at least one of VISTA, CD11 b, CD33, CD4, and CD8 in a biological sample of said subject; and
b) comparing the level of expression of PSGL-1 and at least one of VISTA, CD11b, CD33, CD4, and CD8 of step a) with a reference level;
   wherein an increase in the assayed level of PSGL-1 in step (b) compared to the reference level is indicative of a VISTA-mediated disease, disorder or condition.

The invention also relates to an in vitro method for diagnosing a VISTA-mediated cancer in a subject, said method comprising the steps of:
a) determining the level of expression of PSGL-1 and at least one of VISTA, CD11 b, CD33, CD4, and CD8 in a biological sample of said subject; and
b) comparing the level of expression of PSGL-1 and at least one of VISTA, CD11b, CD33, CD4, and CD8 of step a) with a reference level;
   wherein an increase in the assayed level of PSGL-1 in step (b) compared to the reference level is indicative of a VISTA-mediated disease, disorder or condition.

A more definitive diagnosis of a VISTA-mediated disease, disorder or condition may allow health professionals to employ preventative measures or aggressive treatment earlier thereby preventing the development or further progression of the VISTA-mediated disease, disorder or condition.

### Identification of Patients Susceptible to Respond to Anti-Vista Therapeutic Agents

The above data indicate that PSGL-1 is a dependable biomarker for diagnosing a VISTA-mediated disorder, such as a VISTA-mediated cancer. Patients thus identified are susceptible to respond to anti-VISTA therapeutic agents.

In another aspect, the invention relates to an in vitro method for identifying tumor patients which are susceptible to be treated by an anti-VISTA therapeutic agent. Advantageously, said patients express PSGL-1, notably in immune infiltrates, and the expression of PSGL-1 indicates that said patients are susceptible to be treated by an anti-VISTA therapeutic agent.

In a first embodiment, the present invention relates to an in vitro method of diagnosing in a patient a cancer which is susceptible to treatment with a VISTA-blocking agent, said method comprising the steps of:
a) determining the level of expression of PSGL-1 in a biological sample of said patient; and
b) comparing the level of expression of step a) with a reference level; and
c) diagnosing that the cancer is susceptible to treatment with a VISTA-blocking agent from said comparison.

In another embodiment, said reference level is the level of expression of PSGL-1 in a second biological sample from a second patient having the same VISTA-mediated cancer as the first patient, wherein the second patient is responsive to the treatment. In a preferred embodiment, a similar level of expression of PSGL-1 in the first biological sample compared to the level of expression of PSGL-1 in the second biological sample indicates that the first patient will be responsive to treatment.

In another embodiment, step a) comprises determining the level of expression of PSGL-1 and at least one of VISTA, CD11 b, CD33, CD4, and CD8 in said biological sample, preferably by immune infiltrates. Advantageously, the level of expression of PSGL-1 and at least one of VISTA, CD11b, CD33, CD4, and CD8, or the relative expression levels thereof, in the first biological sample with the level of expression of PSGL-1 is compared with at least one of VISTA, CD11b, CD33, CD4, and CD8, or the relative expression levels thereof, in a second biological sample from a second patient having the same VISTA-mediated cancer as the first patient, wherein the second patient is responsive to the treatment. In a preferred embodiment, a similar level of expression of PSGL-1 and at least one of VISTA, CD11b, CD33, CD4, and CD8, or the relative expression levels thereof, in the first biological sample compared to the level of expression of PSGL-1 and at least one of VISTA, CD11b, CD33, CD4, and CD8, or the relative expression levels thereof, in the second biological sample indicates that the first patient will be responsive to treatment.

### Measuring PSGL-1 Expression

PSGL-1 expression can be measured by any means available to the person of skills in the art. The expression of PSGL-1 can thus be measured by measuring the level of a PSGL-1 nucleic acid (e.g., PSGL-1 mRNA or the corresponding cDNA) or by measuring the level of the PSGL-1 protein.

In this case, the method according to the invention may comprise one or a plurality of intermediate steps between sampling the biological sample and measuring the expression of PSGL-1, said steps corresponding to the extraction from said biological sample of an mRNA sample (or of the corresponding cDNA) or a protein sample. The preparation or extraction of mRNA (and the retrotranscription thereof to cDNA) or proteins from a cell sample are merely routine procedures well-known to those skilled in the art.

Once the mRNA (or corresponding cDNA) or protein sample is obtained, the expression of PSGL-1, in respect of either the mRNA (i.e., in all the mRNA or cDNA present in the sample), or proteins (i.e., in all the proteins present in the sample), may be measured. The method used for this purpose is then dependent on the type of transformation (mRNA, cDNA or protein) and the type of sample available.

When the expression of the marker is measured in respect of mRNA (or corresponding cDNA), any method commonly used by those skilled in the art may be applied. These technologies for analyzing the level of gene expression, such as for example transcriptome analysis, include well-known methods such as PCR (Polymerase Chain Reaction, if using DNA), RT-PCR (Reverse Transcription-PCR, if using RNA) or quantitative RT-PCR or nucleic acid arrays (including DNA arrays and oligonucleotide arrays) for a greater throughput.

The term “nucleic acid arrays” as used herein refers to a plurality of different nucleic acid probes attached to a substrate, which may be a microchip, a glass slide, or a bead having the size of a microsphere. The microchip may consist of polymers, plastics, resins, polysaccharides, silica or a material based on silica, carbon, metals, inorganic glass, or nitrocellulose.

The probes may be nucleic acids such as cDNA (“cDNA array”), mRNA (“mRNA array”) or oligonucleotides (“oligonucleotide array”), said oligonucleotides typically being suitable for having a length between approximately 25 and 60 nucleotides.

To determine the expression profile of a particular gene, a nucleic acid corresponding to all or part of said gene is labelled, then placed in contact with the array under hybridization conditions, resulting in the formation of complexes between said labelled target nucleic acid and the probes attached to the chip surface which are complementary to this nucleic acid. The presence of labelled hybridized complexes is then detected.

These technologies are suitable for monitoring the level of expression of one gene in particular or of a plurality of genes or even all the genes of the genome (full genome or full transcriptome) in a biological sample (cells, tissues, etc.).

In a preferred embodiment, the expression profile is determined using quantitative PCR. Quantitative, or real-time, PCR is a well-known and easily available technology for those skilled in the art and does not need a precise description.

In a particular embodiment, which should not be considered as limiting the scope of the invention, the determination of the expression profile using quantitative PCR may be performed as follows. Briefly, the real-time PCR reactions are carried out using the TaqMan Universal PCR Master Mix (Applied Biosystems). 6 μL cDNA is added to a 9 μL PCR mixture containing 7.5 μL TaqMan Universal PCR Master Mix, 0.75 μL of a 20× mixture of probe and primers and 0.75 μl water. The reaction consisted of one initiating step of 2 min at 50 deg. C., followed by 10 min at 95 deg. C., and 40 cycles of amplification including 15 sec at 95 deg. C. and 1 min at 60 deg. C. The reaction and data acquisition can be performed using the ABI PRISM 7900 Sequence Detection System (Applied Biosystems). The number of template transcript molecules in a sample is determined by recording the amplification cycle in the exponential phase (cycle threshold or C_{T}), at which time the fluorescence signal can be detected above background fluorescence. Thus, the starting number of template transcript molecules is inversely related to C_{T}.

In another preferred embodiment, the expression profile is determined by the use of a nucleic microarray.

The present invention further relates to a microarray dedicated to the implementation of the methods according to the invention, comprising at most 500, preferably at most 300, at most 200, more preferably at most 150, at most 100, even more preferably at most 75, at most 50, at most 40, at most 30, at most 20, at most 10 distinct probes, at least 1 of which specifically binds to PSGL-1 mRNA (or corresponding cDNA) or protein. In a preferred embodiment, said microarray is a nucleic acid microarray, comprising at most 500, preferably at most 300, at most 200, more preferably at most 150, at most 100, even more preferably at most 75, at most 50, at most 40, at most 30, at most 20, at most 10 distinct probes (thus excluding for instance pangenomic microarrays), at least 1 of which specifically hybridizes to PSGL-1 mRNA (or corresponding cDNA). Said microarray may also contain at least one probe which specifically hybridizes to a housekeeping gene in addition to the probe specifically hybridizing to PSGL-1. For example, the housekeeping gene is the beta-2-microglobulin gene.

Alternatively, it is possible to use any current or future technology suitable for determining gene expression on the basis of the quantity of mRNA in the sample. For example, those skilled in the art can measure the expression of a gene by hybridization with a labelled nucleic acid probe, such as for example by means of Northern Blot (for mRNA) or Southern Blot (for cDNA), but also using techniques such as the serial analysis of gene expression (SAGE) method and the derivatives thereof, such as LongSAGE, SuperSAGE, DeepSAGE, etc.

It is also possible to use tissue microarrays (also known as TMAs) as a starting material. TMAs consist of paraffin blocks in which up to 1000 separate tissue cores are assembled in array fashion to allow multiplex histological analysis. In the tissue microarray technique, a hollow needle is used to remove tissue cores as small as 0.6 mm in diameter from regions of interest in paraffin-embedded tissues such as clinical biopsies or tumor samples. These tissue cores are then inserted in a recipient paraffin block in a precisely spaced, array pattern. Sections from this block are cut using a microtome, mounted on a microscope slide and then analyzed by any method of standard histological analysis. Each microarray block can be cut into 100-500 sections, which can be subjected to independent tests. Tests commonly employed in tissue microarray include immunohistochemistry, and fluorescent in situ hybridization. For analysis at the mRNA level, tissue microarray technology may be coupled to fluorescent in situ hybridization. An example of in situ hybridization using the RNAscope 2.5 (Advanced Cell Diagnostics, Hayward, Calif.) is shown in the examples.

Finally, mass parallel sequencing can be used to determine the quantity of mRNA in the sample (RNA-Seq or “Whole Transcriptome Shotgun Sequencing”). For this purpose, a plurality of mass parallel sequencing methods are available. Such methods are described in, for example, U.S. Pat. Nos. 4,882,127, 4,849,077; 7,556,922; 6,723,513; WO 03/066896; WO 2007/111924 US 2008/0020392; WO 2006/084132; US 2009/0186349; US 2009/0181860; US 2009/0181385; US 2006/0275782; EP-B1-1141399; Shendure [symbol] Ji, Nat Biotechnol., 26(10): 1135-45 (2008); Pihlak et al., Nat Biotechnol., 26(6): 676-684 (2008); Fuller et al., Nature Biotechnol., 27(11): 1013-1023 (2009); Mardis, Genome Med., 1(4): 40 (2009); Metzker, Nature Rev. Genet., 11(1): 31-46 (2010).

When the expression of the marker is measured in respect to protein, it is possible to use specific antibodies against PSGL-1. The binding of the anti-PSGL-1 antibody may be detected and/or quantified and/or determined by various assays available to the skilled artisan, such as e.g. immunoprecipitation, immunochemistry (IHC), Western Blot, Dot Blot, ELISA, ELISPOT, protein arrays, antibody arrays, or tissue arrays coupled with immunohistochemistry. Other techniques which may be used include Fluorescence Activated Cell Sorting (FACS), FRET or BRET techniques, microscopy or histochemistry methods, particularly including confocal microscopy and electron microscopy methods, methods based on the use of one or a plurality of excitation wavelengths and a suitable optical method, such as an electrochemical method (voltammetry and amperometry techniques), atomic force microscopy, and radiofrequency methods, such as multipolar, confocal and non-confocal resonance spectroscopy, detection of fluorescence, luminescence, chemoluminescence, absorbance, reflectance, transmittance, and birefringence or refractive index (for example, by means of surface plasmon resonance, by ellipsometry, by means of the resonant mirror method, etc.), flow cytometry, radioisotopic or magnetic resonance imaging, analysis by means of polyacrylamide gel electrophoresis (SDS-PAGE); by means of HPLC-Mass spectrophotometry, by means of liquid chromatography/mass spectrophotometry/mass spectrometry (LC-MS/MS). All these techniques are well-known to those skilled in the art and it is not necessary to detail them herein.

Although any of the above methods is suitable for carrying out the present methods, FACS, ELISA, ELISPOT, western blotting and IHC can be mentioned in particular. Preferred methods include ELISPOT, FACS and IHC.

### Determination of the PSGL-1 Status of Tumor

Determination of the binding of a reagent to PSGL-1 (as described above) allows for the determination of the PSGL-1 status of the tumor to be treated. The PSGL-1 status can be determined by any method or technique known or currently used by the person skilled in the art, generally based on the determination of the expression level of PSGL-1. Based on the PSGL-1 status of the tumor; it is then possible to predict whether a patient will respond to an anti-VISTA therapeutic agent.

Recently, it has become apparent that that the immunological data (the type, density, and location of immune cells within the tumor samples) are a better predictor of patient survival than the histopathological methods currently used to stage colorectal cancer.

Furthermore, increasing evidence from clinical trials supports the potential of therapies that target immune activity in certain types of cancer (Robert et al., Stagg et al.). This has led to the development of more standardized methods of characterizing tumor immune infiltrate in cancers such as the “immune score” that aims to quantify the in situ immune infiltrate in addition to standardized clinical parameters to aid prognostication and patient selection for immunotherapy in various cancers (see e.g. Galon et al., J Pathol 232(2): 199-209 (2014); Galon et al., J Transl Med 14: 273 (2016)).

The method of detecting or diagnosing a VISTA-mediated cancer described herein thus includes determining the PSGL-1 score of the tumor.

According to this embodiment, the method comprises the steps of:
a) contacting a biological sample of said subject with a reagent capable of binding PSGL-1 protein or nucleic acid;
b) quantifying the binding of said reagent with said biological sample; and
c) scoring the tumoral cells by comparing the quantified level obtained in step a) to an appropriate scale based on two parameters which are the intensity of the staining and the percentage of positive cells.

In a preferred embodiment, step b) comprises quantifying the binding of said reagent with PSGL-1 in immune infiltrates of the tumor microenvironment in said biological sample.

According to this preferred embodiment, the method comprises the steps of:
a) contacting a biological sample of said subject with a reagent capable of binding PSGL-1 protein or nucleic acid;
b) quantifying the binding of said reagent with said biological sample; and
c) scoring the tumor immune cells by comparing the quantified level obtained in step a) to an appropriate scale based on two parameters which are the intensity of the staining and the percentage of positive cells.

The tumor immune cells (or immune infiltrates) comprise the immune cells present in the tumor microenvironment, notably the immunosuppressive cells of the tumor microenvironment, like some macrophages, monocytes etc. In a preferred embodiment, immune infiltrates include lymphocytes (e.g., T cells, B-cells, natural killer (NK) cells), dendritic cells, mast cells, and macrophages. Accordingly, in this embodiment step b) comprises quantifying the binding of said reagent with PSGL-1 on lymphocytes (e.g., T cells, B-cells, natural killer (NK) cells), dendritic cells, mast cells, and macrophages present in the tumor microenvironment in said biological sample.

Any conventional hazard analysis method may be used to estimate the prognostic value of PSGL-1. Representative analysis methods include Cox regression analysis, which is a semiparametric method for modeling survival or time-to-event data in the presence of censored cases (Hosmer and Lemeshow, 1999; Cox, 1972). In contrast to other survival analyses, e.g. Life Tables or Kaplan-Meyer, Cox allows the inclusion of predictor variables (covariates) in the models. Using a convention analysis method, e.g., Cox one may be able to test hypotheses regarding the correlation of PSGL-1 expression status of in a primary tumor to time-to-onset of either disease relapse (disease-free survival time, or time to metastatic disease), or time to death from the disease (overall survival time). Cox regression analysis is also known as Cox proportional hazard analysis. This method is standard for testing the prognostic value of a tumor marker on patient survival time. When used in multivariate mode, the effect of several covariates are tested in parallel so that individual covariates that have independent prognostic value can be identified, i.e. the most useful markers. The term negative or positive “PSGL-1 status” can also be referred as [PSGL-1 (−)] or [PSGL-1 (+)].

A sample may be “scored” during the diagnosis or monitoring of cancer. In its simplest form, scoring may be categorical negative or positive as judged by visual examination of samples by immunohistochemistry. More quantitative scoring involves judging the two parameters intensity of staining and the proportion of stained (“positive”) cells that are sampled.

In an embodiment, to ensure standardization, samples may be scored for PSGL-1 expression levels on different scales, most of them being based on an assessment of the intensity of the reaction product and the percentage of positive cells (Payne et al., Predictive markers in breast cancer—the present, Histopathology 2008, 52, 82-90).

In another embodiment, said scoring comprises using an appropriate scale based on the intensity of the staining and the percentage of positive cells.

As a first example, by analogy with the Quick Allred scoring for IHC assessment of oestrogen receptor and progesterone receptor, samples may be scored for PSGL-1 expression levels on a global scale from 0 to 8 combining scores for intensity of reactivity and for the proportion of cells stained (Harvey J M, Clarck G M, Osborne C K, Allred D C; J. Clin. Oncol. 1999; 17; 1474-1481). More particularly, the first criteria of intensity of reactivity is scored on a scale from 0 to 3, 0 corresponding to “No reactivity” and 3 corresponding to “Strong reactivity”. The second criteria of proportion reactive is scored on a scale from 0 to 5, 0 corresponding to “No reactivity” and 5 to “67-100% proportion reactive”. The intensity of the reactivity score and the proportion reactive score are then summed to produce total score of 0 through 8. A total score of 0-2 is regarded as negative while a total score of 3-8 is regarded as positive.

According to this scale, the terms negative or positive “PSGL-1 status” of tumors used in the present description refers to levels of expression of PSGL-1 that correspond to scores 0-2 or 3-8 on the Allred scale, respectively.

Table 2 hereinafter illustrates the guidelines for interpreting IHC results according to Allred method.

TABLE 2

| Intensity of immunoreactivity | Score 1 | Proportion reactive | Score 2 |
|---|---|---|---|
| No reactivity | 0 | No reactivity | 0 |
| Weak reactivity | 1 | <1% | 1 |
| Moderate reactivity | 2 | 1-10% | 2 |
| Strong reactivity | 3 | 11-33% | 3 |
| | — | 34-66% | 4 |
| | — | 67-100% | 5 |

| Total Score | |
|---|---|
| (Score 1 + Score 2) | Interpretation |
| 0-2 | Negative |
| 3-8 | Positive |

According to the invention, the said appropriate scale may be a scale of 0 to 8 wherein no reactivity is scored 0, and a strong reactivity in a proportion of 67-100% proportion reactive is scored 8

In other words, it is described a process of determining in vitro or ex vivo the status of a tumor from a subject, wherein said process comprises the steps of (a) scoring a tumor from a subject according to the Allred scale; and (b) determining that the status of the tumor is [PSGL-1(+)] with an Allred score of 3 to 8; or (c) determining that the status of the tumor is [PSGL-1(−)] with an Allred score of 0 to 2.

In a particular aspect of the invention, a tumor is [PSGL-1 (+)] with an Allred score of 3.

In a particular aspect of the invention, a tumor is [PSGL-1 (+)] with an Allred score of 4.

In a particular aspect of the invention, a tumor is [PSGL-1 (+)] with an Allred score of 5.

In a particular aspect of the invention, a tumor is [PSGL-1 (+)] with an Allred score of 6.

In a particular aspect of the invention, a tumor is [PSGL-1 (+)] with an Allred score of 7.

In a particular aspect of the invention, a tumor is [PSGL-1 (+)] with an Allred score of 8.

In another particular aspect of the invention, a tumor is [PSGL-1 (+)] with an Allred score of 3 to 8.

Another particular method herein described for determining in vitro or ex vivo the PSGL-1 status of tumoral cells in a subject, is characterized in that it comprises the steps of:

(a) scoring PSGL-1 tumoral cells as above described; and

(b) determining that the PSGL-1 status of tumoral cells is [PSGL-1(+)] with a score of 3 to 8; or

(c) determining that the PSGL-1 status of tumoral cells is [PSGL-1(−)] with a score of 0 to 2.

Another particular method herein described for determining in vitro or ex vivo the PSGL-1 status of tumor immune cells in a subject, is characterized in that it comprises the steps of:

(a) scoring PSGL-1 tumor immune cell as above described; and

(b) determining that the PSGL-1 status of tumor immune cells is [PSGL-1(+)] with a score of 3 to 8; or

(c) determining that the PSGL-1 status of tumor immune cells is [PSGL-1(−)] with a score of 0 to 2.

In a preferred embodiment, tumor immune cells (i.e. immune infiltrates) include lymphocytes (e.g., T cells, B-cells, natural killer (NK) cells), dendritic cells, mast cells, and macrophages. Accordingly, in this embodiment step a) comprises quantifying the binding of said reagent with PSGL-1 on lymphocytes (e.g., T cells, B-cells, natural killer (NK) cells), dendritic cells, mast cells, and macrophages present in the tumor microenvironment in said biological sample.

As a second example, by analogy with the conventional scoring for IHC assessment of HER-2 receptor for example, samples may be scored for PSGL-1 expression levels on a somewhat simpler scoring method integrating the intensity of staining (preferentially membranous staining) and the proportion of cells that display staining into a combined scale from 0 to 3+.

In this scale, referred as the simplified scale, 0 and 1+ are negative whereas 2+ and 3+ represents positive staining. Nevertheless, scores 1+-3+ can be recoded as positive because each positive score may be associated with significantly higher risk for relapse and fatal disease when compared to score 0 (negative), but increasing intensity among the positive scores may provide additional risk reduction.

Generally speaking, the terms negative or positive “PSGL-1 status” of tumors used in the present description refers to levels of expression of PSGL-1 that correspond to scores 0-1+ or 2+-3+ on the simplified scale, respectively. Only complete circumferential membranous reactivity of the invasive tumor should be considered and often resembled a “chicken wire” appearance. Under current guidelines, samples scored as borderline (score of 2+ or 3+) for PSGL-1 are required to undergo further assessment. The IHC analysis should be rejected, and either repeated or tested by FISH or any other method if, as non limitative example, controls are not as expected, artifacts involve most of the sample and the sample has strong membranous positivity of normal breast ducts (internal controls) suggesting excessive antigen retrieval.

For more clarity, Table 3 hereinafter summarizes these parameters.

TABLE 3

| PSGL-1 status | IHC description |
|---|---|
| 0 | No reactivity or membranous reactivity |
| | in less than 10% of tumor cells or |
| | tumor immune cells |
| 1⁺ | Faint/barely perceptible membranous |
| | reactivity is detected in more than |
| | 10% of tumor cells or tumor immune cells. |
| | The cells are immunoreactive only in part |
| | of the membrane. |
| 2⁺ | Weak to moderate complete membranous |
| | reactivity is seen in more than 10% |
| | of tumor cells or tumor immune cells. |
| 3⁺ | Strong complete reactivity is seen in |
| | more than 10% of tumor cells or tumor immune cells. |

The appropriate scale may be a scale of 0 to 3′ wherein no membranous reactivity of tumor cells or tumor immune cells is scored 0 and strong complete reactivity in more than 10% of tumor cells is scored 3′.

In more details, as above described, said appropriate scale is a scale of 0 to 3 wherein no membranous reactivity of tumor cells or tumor immune cells is scored 0; faint perceptible membranous reactivity in more than 10% of tumor cells or tumor immune cells is scored 1+; weak to moderate complete membranous reactivity in more than 10% of tumor cells or tumor immune cells is scored 2+; and strong complete reactivity in more than 10% of tumor cells or tumor immune cells is scored 3+.

In other words, it is described a process of determining in vitro or ex vivo the status of a tumor from a subject, wherein said process comprises the steps of (a) scoring a tumor from a subject according to the simplified scale as above described; and (b) determining that the status of the tumor is [PSGL-1(+)] with a score of 2+ or 3+; or (c) determining that the status of the tumor is [PSGL-1(−)] with a score of 0 or 1+.

In a particular aspect of the invention, a tumor is [PSGL-1 (+)] with a score of 2+.

In a particular aspect of the invention, a tumor is [PSGL-1 (+)] with a score of 3+.

In another particular aspect of the invention, a tumor is [PSGL-1 (+)] with a score of 2+ or 3+.

In another embodiment, the method for determining in vitro or ex vivo the PSGL-1 status tumoral cells in a subject may comprise the steps of:

(a) scoring PSGL-1 tumor cells from the said subject according to the method described above; and

(b) determining that the PSGL-1 status of tumor cells is [PSGL-1(+)] with a score of 2⁺ or 3⁺; or

(c) determining that the PSGL-1 status of tumor cells is [PSGL-1(−)] with a score of 0 or 1⁺.

In another embodiment, the method for determining in vitro or ex vivo the PSGL-1 status tumor immune cells in a subject may comprise the steps of:

(a) scoring PSGL-1 tumor immune cells from the said subject according to the method described above; and

(b) determining that the PSGL-1 status of tumor immune cells is [PSGL-1(+)] with a score of 2⁺ or 3⁺; or

(c) determining that the PSGL-1 status of tumor immune cells is [PSGL-1(−)] with a score of 0 or 1⁺.

In a preferred embodiment, tumor immune cells (i.e. immune infiltrates) include lymphocytes (e.g., T cells, B-cells, natural killer (NK) cells), dendritic cells, mast cells, and macrophages. Accordingly, in this embodiment step a) comprises quantifying the binding of said reagent with PSGL-1 on lymphocytes (e.g., T cells, B-cells, natural killer (NK) cells), dendritic cells, mast cells, and macrophages present in the tumor microenvironment in said biological sample.

Generally, the results of a test or assay can be presented in any of a variety of formats. The results can be presented qualitatively. For example, the test report may indicate only whether or not a particular polypeptide was detected, perhaps also with an indication of the limits of detection. The results may be displayed as semi-quantitative. For example, various ranges may be defined, and the ranges may be assigned a score (e.g., 0 to 3+ or 0 to 8 depending on the used scale) that provides a certain degree of quantitative information. Such a score may reflect various factors, e.g., the number of cells in which PSGL-1 is detected, the intensity of the signal (which may indicate the level of expression of PSGL-1 or PSGL-1-bearing cells), etc. The results may be displayed in a quantitative way, e.g., as a percentage of cells in which PSGL-1 is detected, as a protein concentration, etc.

As will be appreciated by one of ordinary skill in the art, the type of output provided by a test will vary depending upon the technical limitations of the test and the biological significance associated with detection of the polypeptide. For example, in the case of certain polypeptides a purely qualitative output (e.g., whether or not the polypeptide is detected at a certain detection level) provides significant information. In other cases, a more quantitative output (e.g., a ratio of the level of expression of the polypeptide in the sample being tested versus the normal level) is necessary.

### Anti-PSGL-1 Antibodies

The antibodies for use in the present methods are antibodies that bind to PSGL-1, including a PSGL-1 polypeptide, a PSGL-1 polypeptide fragment, or a PSGL-1 epitope. Anti-PSGL-1 antibodies include humanized anti-PSGL-1 antibodies. Also provided are antibodies (e.g., humanized anti-PSGL-1 antibodies) that competitively block an anti-PSGL-1 antibody provided herein from binding to a PSGL-1 polypeptide.

The present disclosure also provides antibodies that binds PSGL-1 and agonize or antagonize the interaction between PSGL-1 and VISTA. Preferably, the anti-PSGL-1 antibody inhibits or blocks the binding of PSGL-1 to VISTA, notably to the extracellular domain of VISTA. In some embodiments, the anti-PSGL-1 antibody inhibits or blocks the binding of a VISTA-expressing cell to a PSGL-1-expressing T cell, such as, e.g., a myeloid cell, a dendritic cell, a macrophage or a T cell. In some embodiments, the anti-PSGL-1 antibody does not block or inhibit the binding of PSGL-1 to P-selectin, L-selectin or E-selectin.

The anti-PSGL-1 antibodies (e.g., humanized anti-PSGL-1 antibodies) provided herein can also be conjugated or recombinantly fused to a diagnostic agent, detectable agent or therapeutic agent (e.g., antibody-drug conjugate). For example, a detectable agent may be a detectable probe. Further provided are compositions, including pharmaceutical compositions, comprising an anti-PSGL-1 antibody (e.g., a humanized anti-PSGL-1 antibody).

Antibodies provided herein that bind to an antigen, e.g. PSGL-1, can be produced by any method known in the art for the synthesis of antibodies, in particular, by chemical synthesis or by recombinant expression techniques. For example, several anti-PSGL-1 antibodies and methods of producing such antibodies have been previously described (see, e.g., WO 2005/110475, WO 2003/013603; U.S. Patent Application Publication Nos. 2009/0198044, 2005/0266003, 2009/0285812, 2013/0011391, and 2015/0183870; and U.S. Pat. Nos. 7,833,530, and 8,361,472).

Polyclonal antibodies that bind to an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981) (the references incorporated by reference in their entireties). The term “monoclonal antibody” as used herein is not limited to antibodies produced through hybridoma technology. Other exemplary methods of producing monoclonal antibodies are discussed elsewhere herein, such as e.g., use of the KM Mouse™. Additional exemplary methods of producing monoclonal antibodies are provided in the Examples herein. Alternatively, it is also possible to use an anti-PSGL-1 antibody such as e.g., the antibodies described in WO 2003/013603, WO 2005/110475, WO 2009/140623, Dimitroff et al., Cancer Res, 65(13): 5750-60 (2005), Veerman et al., Nature Immunol 8(5):532-9 (2007), Tinocco et al., Immunity 44: 1190-1203 (2016).

Methods for producing and screening for specific antibodies using hybridoma technology are routine and well known in the art. Briefly, mice can be immunized with a PSGL-1 antigen and once an immune response is detected, e.g., antibodies specific for PSGL-1 antigen are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution.

Additionally, a RIMMS (repetitive immunization multiple sites) technique can be used to immunize an animal (Kilptrack et al., 1997 Hybridoma 16:381-9, incorporated by reference in its entirety). The hybridoma clones are then assayed by methods known in the art for cells that secrete antibodies capable of binding a given polypeptide. Ascites fluid, which generally contains high levels of antibodies, can be generated by immunizing mice with positive hybridoma clones.

Accordingly, also provided herein are methods of generating antibodies by culturing a hybridoma cell secreting a modified antibody provided herein wherein, in some embodiments, the hybridoma is generated by fusing splenocytes isolated from a mouse immunized with PSGL-1, including a PSGL-1 polypeptide, a PSGL-1 polypeptide fragment or a PSGL-1 epitope, with myeloma cells and then screening the hybridomas resulting from the fusion for hybridoma clones that secrete an antibody able to bind to PSGL-1.

Anti-PSGL-1 antibodies capable of modulating (e.g., increasing or inhibiting) the interaction between PSGL-1 and VISTA can be identified by any method known to the person of skills in the art. Examples of assays for detecting and measuring the interaction between PSGL-1 and VISTA are described in the Experimental Section. Any of these assays may be used to test whether an anti-PSGL-1 antibody can modulate the interaction between PSGL-1 and VISTA.

Antibody fragments which recognize (e.g., bind to) PSGL-1 may be generated by any technique known to those of skill in the art. For example, Fab and F(ab′)₂ fragments provided herein may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. Further, the antibodies provided herein can also be generated using various phage display methods known in the art.

For example, antibodies can also be generated using various phage display methods. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies provided herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT/GB91/01134; WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (the references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, human or chimeric antibodies can be used. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and WO 98/46645, WO 98/50433, WO 98/24893, WO 98/16654, WO 96/34096, WO 96/33735, and WO 91/10741; each of which is incorporated herein by reference in its entirety.

In some embodiments, human antibodies are produced. Human antibodies and/or fully human antibodies can be produced using any method known in the art. For example, transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered nonfunctional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of the polypeptide. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. Other methods are detailed in the Examples herein. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)₂, Fabc, Fv) in which all or substantially all of the CDR regions correspond to those of a non-human immunoglobulin (e.g., donor antibody) and all or substantially all of the framework regions are those of a human immunoglobulin consensus sequence. In some embodiments, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibits cytotoxic activity, and the class is typically IgG1. Where such cytotoxic activity is not desirable, the constant domain may be of the IgG2 class. Examples of VL and VH constant domains that can be used in some embodiments include, but are not limited to, C-kappa and C-gamma-1 (nG1m) described in Johnson et al. (1997) J. Infect. Dis. 176, 1215-1224 and those described in U.S. Pat. No. 5,824,307. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental FR and CDR sequences, more often 90%, or greater than 95%. Humanized antibodies can be produced using variety of techniques known in the art, including but not limited to, CDR-grafting (EP 239 400; WO 91/09967; and U.S. Pat. Nos. 5,225,539, 5,530,101, and 5,585,089), veneering or resurfacing (EP 592 106 and EP 519 596; Padlan, 1991, Molecular Immunology 28(4/5):489-498; Studnicka et al., 1994, Protein Engineering 7(6):805-814; and Roguska et al., 1994, Proc Natl Acad Sci 91:969-973), chain shuffling (U.S. Pat. No. 5,565,332), and techniques disclosed in, e.g., U.S. Pat. Nos. 6,407,213, 5,766,886, WO 93/17105, Tan et al., J. Immunol. 169:111925 (2002), Caldas et al., Protein Eng. 13(5):353-60 (2000), Morea et al., Methods 20(3):26779 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1, which is incorporated by reference herein in its entirety. Often, framework residues in the framework regions will be substituted with the corresponding residue from the CDR donor antibody to alter (e.g., improve) antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Reichmann et al., 1988, Nature 332:323, which are incorporated herein by reference in their entireties.)

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and Nos. WO 94/04678, WO 94/25591, and WO 01/44301, each of which is incorporated herein by reference in its entirety.

Further, the antibodies that bind to PSGL-1 can, in turn, be utilized to generate anti-idiotype antibodies that “mimic” an antigen using techniques well known to those skilled in the art. (See, e.g., Greenspan Et Bona, 1989, FASEB J. 7(5):437-444; Nissinoff, 1991, J. Immunol. 147(8):2429-2438).

Antibodies provided herein include, but are not limited to, synthetic antibodies, monoclonal antibodies, recombinantly produced antibodies, multispecific antibodies (including bi-specific antibodies), human antibodies, humanized antibodies, camelized antibodies, chimeric antibodies, intrabodies, anti-idiotypic (anti-Id) antibodies, and functional fragments of any of the above. Non-limiting examples of functional fragments include single-chain Fvs (scFv) (e.g., including monospecific, bispecific, etc.), Fab fragments, F(ab′) fragments, F(ab)₂ fragments, F(ab′)₂ fragments, disulfide-linked Fvs (sdFv), Fd fragments, Fv fragments, diabody, triabody, tetrabody and minibody.

In particular, antibodies provided herein include immunoglobulin molecules and immunologically active portions of immunoglobulin molecules, e.g., molecules that contain an antigen binding site that bind to PSGL-1 (e.g., PSGL-1 polypeptide, PSGL-1 polypeptide fragment, PSGL-1 epitope). The immunoglobulin molecules provided herein can be of any type (e.g., IgG, IgE, IgM, IgD, IgA and IgY), class (e.g., IgG1, IgG2, IgG3, IgG4, IgA1 and IgA2) or subclass of immunoglobulin molecule.

Variants and derivatives of antibodies include antibody functional fragments that retain the ability to bind to PSGL-1 (e.g., PSGL-1 polypeptide, PSGL-1 polypeptide fragment, PSGL-1 epitope). Exemplary functional fragments include Fab fragments (an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)₂ (two Fab′ molecules joined by interchain disulfide bonds in the hinge regions of the heavy chains; the Fab′ molecules may be directed toward the same or different epitopes); a bispecific Fab (a Fab molecule having two antigen binding domains, each of which may be directed to a different epitope); a single chain Fab chain comprising a variable region, also known as, a sFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific sFv (a sFv or a dsFv molecule having two antigen-binding domains, each of which may be directed to a different epitope); a diabody (a dimerized sFv formed when the VH domain of a first sFv assembles with the VL domain of a second sFv and the VL domain of the first sFv assembles with the VH domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes). Derivatives of antibodies also include one or more CDR sequences of an antibody combining site. The CDR sequences may be linked together on a scaffold when two or more CDR sequences are present. In some embodiments, the antibody comprises a single-chain Fv (“scFv”). scFvs are antibody fragments comprising the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFvs see Pluckthun in The Pharmacology of Monoclonal Antibodies, vol. 113, Rosenburg and Moore eds. Springer-Verlag, New York, pp. 269-315 (1994).

The antibodies provided herein may be monospecific, bispecific, trispecific or of greater multispecificity. Multispecific antibodies may be specific for different epitopes of a PSGL-1 polypeptide or may be specific for both a PSGL-1 polypeptide as well as for a heterologous epitope, such as a heterologous polypeptide or solid support material. In some embodiments, the antibodies provided herein are monospecific for a given epitope of a PSGL-1 polypeptide and do not bind to other epitopes.

Also provided herein are fusion proteins comprising an antibody provided herein that binds to a PSGL-1 and a heterologous polypeptide. In some embodiments, the heterologous polypeptide to which the antibody is fused is useful for targeting the antibody to cells having cell surface-expressed PSGL-1.

Also provided herein are panels of antibodies that bind to a PSGL-1. In some embodiments, panels of antibodies have different association rate constants different dissociation rate constants, different affinities for PSGL-1, and/or different specificities for a PSGL-1. In some embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

### Diagnostic Use of PSGL-1 Binding Reagents

Anti-PSGL-1 antibodies provided herein can be used to assay PSGL-1 levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin.

Also provided herein is detection and diagnosis of a VISTA-mediated disease, disorder or condition in a human. In some embodiment, diagnosis comprises: a) administering (for example, parenterally, subcutaneously, or intraperitoneally) to a subject an effective amount of a labeled antibody that binds to a PSGL-1; b) waiting for a time interval following the administering for permitting the labeled antibody to preferentially concentrate at sites in the subject where the PSGL-1 is expressed (and for unbound labeled molecule to be cleared to background level); c) determining background level; and d) detecting the labeled antibody in the subject, such that detection of labeled antibody above the background level indicates that the subject has a VISTA-mediated disease, disorder or condition. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to preferentially concentrate at sites in the subject and for unbound labeled antibody to be cleared to background level is 6 to 48 hours or 6 to 24 hours or 6 to 12 hours. In another embodiment, the time interval following administration is 5 to 20 days or 5 to 10 days.

In some embodiments, monitoring of a VISTA-mediated disease, disorder or condition is carried out by repeating the method for diagnosing the VISTA-mediated disease, disorder or condition, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In some embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### Anti-Vista Therapeutic Agents

In a first embodiment, the anti-VISTA therapeutic agent is an agent which inhibits VISTA checkpoint inhibitor function. Inhibition of VISTA inhibitory function can be performed at the DNA, RNA or protein level. In embodiments, an inhibitory nucleic acid (e.g., a dsRNA, siRNA or shRNA), can be used to inhibit expression of VISTA. In other embodiments, the inhibitor of VISTA inhibitory signal is, a polypeptide e.g., a soluble ligand (e.g., PSGL-1-Fc), or an antibody or antigen-binding fragment thereof (also referred to herein as “an antibody molecule”), that binds to VISTA. Preferably, the anti-VISTA therapeutic agent is an antibody.

Antibodies inhibiting VISTA function are particularly useful for treating cancer. The present inventors have previously described antibodies directed against VISTA which induce strong tumor growth inhibition (see WO 2014/197849 and WO 2016/094837, both incorporated herein by reference). Other anti-VISTA antibodies with anti-cancer properties have also been described in the art (see e.g., WO 2014/039983A1, WO 2015/145360A1, WO 2015/097536, WO 2017/137830, WO 2017/181139, all of which are hereby incorporated by reference in their entireties).

Such highly specific and/or specific anti-VISTA antibodies (referred to herein as “anti-VISTA antibodies”) may be polyclonal (“anti-VISTA PAbs”) or monoclonal (“anti-VISTA MAbs”), although for therapeutic uses and, in some instances, diagnostic or other in vitro uses, monoclonal antibodies are preferred.

In specific embodiments, the antibody is a humanized antibody, a monoclonal antibody, a recombinant antibody, an antigen binding fragment or any combination thereof. In particular embodiments, the antibody is a humanized monoclonal antibody as described in WO 2016/094837 (e.g., 5B, 46A, 97A, 128A, 146C, 208A, 215A, 26A, 164A, 230A, 76E1, 53A, 259A, 33A, 39A, 124A, 175A, 321D, 141A, 51A, 353A, or 305A described therein (e.g., Tables 12-33 of WO 2016/094837) with a V_{H} domain, V_{L} domain, V_{H} CDR1, V_{H} CDR2, V_{H} CDR3, V_{L} CDR1, VL CDR2, and/or VL CDR3), or antigen binding fragment thereof, that binds to a VISTA polypeptide (e.g., a cell surface-expressed or soluble VISTA), a VISTA fragment, or a VISTA epitope.

In other embodiments, the anti-VISTA antibodies used in the method of the invention are antibodies (i) that competitively block (e.g., in a dose-dependent manner) an anti-VISTA antibody as described in WO 2016/094837 from binding to a VISTA polypeptide (e.g., a cell surface-expressed or soluble VISTA), a VISTA fragment, or a VISTA epitope and/or (ii) that bind to a VISTA epitope that is bound by an anti-VISTA antibody (e.g., humanized anti-VISTA antibodies) as described in WO 2016/094837. In other embodiments, the antibody competitively blocks (e.g., in a dose-dependent manner) monoclonal antibody 5B, 46A, 97A, 128A, 146C, 208A, 215A, 26A, 164A, 230A, 76E1, 53A, 259A, 33A, 39A, 124A, 175A, 321D, 141A, 51A, 353A, or 305A described herein (e.g., Tables 12-33) or a humanized variant thereof from binding to a VISTA polypeptide (e.g., a cell surface-expressed or soluble VISTA), a VISTA fragment, or a VISTA epitope. In other embodiments, the antibody binds to a VISTA epitope that is bound (e.g., recognized) by monoclonal antibody 5B, 46A, 97A, 128A, 146C, 208A, 215A, 26A, 164A, 230A, 76E1, 53A, 259A, 33A, 39A, 124A, 175A, 321D, 141A, 51A, 353A, or 305A described in WO 2016/094837 (e.g., Tables 12-33 of WO 2016/094837) or a humanized variant thereof (e.g. humanized anti-VISTA antibodies).

More preferably, the anti-VISTA antibody of the method of the invention is the antibody 26A described in WO 2016/094837. In a first embodiment, this antibody comprises a heavy chain comprising 3 CDRs and light chain comprising 3 CDRs, wherein said CDRS are shown in Table 4. In another embodiment, the anti-VISTA antibody comprises a heavy chain comprising 3 CDRs and light chain comprising 3 CDRs, wherein said CDRS are shown in Table 5.

TABLE 4

| | | Exemplary* | IMGT | Kabat | Chothia | Contact | AbM |
|---|---|---|---|---|---|---|---|
| VH CDR | VH | GFSFTGY | GFSFTGY | GYTMN | GFSFTGY | TGYTMN | GFSFTGY |
| Seq. | CDR1 | TMN | T | (SEQ ID | (SEQ ID | (SEQ ID | TMN |
| | | (SEQ ID | (SEQ ID | NO: 7) | NO: 8) | NO: 9) | (SEQ ID |
| | | NO: 5) | NO: 6) | | | | NO: 5) |
| | VH | LISPYNG | ISPYNGG | LISPYNG | PYNG | WIGLISP | LISPYNG |
| | CDR2 | GTSYNQK | T | GTSYNQK | (SEQ ID | YNGGTS | GTS |
| | | FKG | (SEQ ID | FKG | NO: 12) | (SEQ ID | (SEQ ID |
| | | (SEQ ID | NO: 11) | (SEQ ID | | NO: 13) | NO: 14) |
| | | NO: 10) | | NO: 10) | | | |
| | VH | RAYGYAM | ARRAYGY | RAYGYAM | AYGYAMD | ARRAYGY | RAYGYAM |
| | CDR3 | DY | AMDY | DY | (SEQ ID | AMD | DY |
| | | (SEQ ID | (SEQ ID | (SEQ ID | NO: 17) | (SEQ ID | (SEQ ID |
| | | NO: 15) | NO: 16) | NO: 15) | | NO: 18) | NO: 15) |
| VL CDR | VL | SASSSVS | SSVSY | SASSSVS | SSSVSY | SYMYWY | SASSSVS |
| Seq. | CDR1 | YMY | (SEQ ID | YMY | (SEQ ID | (SEQ ID | YMY |
| | | (SEQ ID | NO: 20) | (SEQ ID | NO: 21) | NO: 22) | (SEQ ID |
| | | NO: 19) | | NO: 19) | | | NO: 19) |
| | VL | DTSNLAS | DTS | DTSNLAS | DTS | LLIYDTS | DTSNLAS |
| | CDR2 | (SEQ ID | (SEQ ID | (SEQ ID | (SEQ ID | NLA | (SEQ ID |
| | | NO: 23) | NO: 24) | NO: 23) | NO: 24) | (SEQ ID | NO: 23) |
| | | | | | | NO: 25) | |
| | VL | QQWSSYP | QQWSSYP | QQWSSYP | WSSYPF | QQWSSYP | QQWSSYP |
| | CDR3 | FT | FT | FT | (SEQ ID | F | FT |
| | | (SEQ ID | (SEQ ID | (SEQ ID | NO: 27) | (SEQ ID | (SEQ ID |
| | | NO: 26) | NO: 26) | NO: 26) | | NO: 28) | NO: 26) |

VH Sequence:
EVQLQQSGPELVKPGASMKISCKASGFSFTGYTMNW
VKQSHVKNLEWIGLISPYNGGTSYNQKFKGKATLTV
DKSSSTAYMELLSLTSEDSAVYY
CARRAYGYAMDYWGQGTSVTVSS (SEQ ID NO: 29)
VL Sequence:
QIVLTQSPAIMSASPGEKVTMTCSASSSVSYMYWYQQ
KPGSSPRLLIYDTSNLASGVPLRFSGSGSGTSYSLTI
SRMEAEDAATYYCQQWSSYPF
TFGSGTKLEIK (SEQ ID NO: 30)

TABLE 5

| | | Exemplary* | IMGT | Kabat | Chothia | Contact | AbM |
|---|---|---|---|---|---|---|---|
| VH CDR | VH | GFSFTG | GFSFTGYT | GYTMN | GFSFTGY | TGYTMN | GFSFTG |
| Seq. | CDR1 | YTMN | (SEQ ID | (SEQ ID | (SEQ ID | (SEQ ID | YTMN |
| | | (SEQ ID | NO: 6) | NO: 7) | NO: 8) | NO: 9) | (SEQ ID |
| | | NO: 5) | | | | | NO: 5) |
| | VH | LISPYD | ISPYDGGT | LISPYD | PYDG | WIGLISP | LISPYD |
| | CDR2 | GGTS | (SEQ ID | GGTSYN | (SEQ ID | YDGGT | GGTS |
| | | YNQKFKG | NO: 32) | QKFKG | NO: 33) | S | (SEQ ID |
| | | (SEQ ID | | (SEQ ID | | (SEQ ID | NO: 35) |
| | | NO: 31) | | NO: 31) | | NO: 34) | |
| | VH | RAYGYA | ARRAYGY | RAYGY | AYGYAMD | ARRAYG | RAYGY |
| | CDR3 | MDY | AMDY | AMDY | (SEQ ID | YAMD | AMDY |
| | | (SEQ ID | (SEQ ID | (SEQ ID | NO: 17) | (SEQ ID | (SEQ ID |
| | | NO: 15) | NO: 16) | NO: 15) | | NO: 18) | NO: 15) |
| VL CDR | VL | SASSSV | SSVSY | SASSSV | SSSVSY | SYMYWY | SASSSV |
| Seq. | CDR1 | SYMY | (SEQ ID | SYMY | (SEQ ID | (SEQ ID | SYMY |
| | | (SEQ ID | NO: 20) | (SEQ ID | NO: 21) | NO: 22) | (SEQ ID |
| | | NO: 19) | | NO: 19) | | | NO: 19) |
| | VL | DTSNLAS | DTS | DTSNLAS | DTS | LLIYDT | DTSNLAS |
| | CDR2 | (SEQ ID | (SEQ ID | (SEQ ID | (SEQ ID | SNLA | (SEQ ID |
| | | NO: 23) | NO: 24) | NO: 23) | NO: 24) | (SEQ ID | NO: 23) |
| | | | | | | NO: 25) | |
| | VL | QQWSSY | QQWSS | QQWSSY | WSSYPF | QQWSSYPF | QQWSS |
| | CDR3 | PFT | YPFT | PFT | (SEQ ID | (SEQ ID | YPFT |
| | | (SEQ ID | (SEQ ID | (SEQ ID | NO: 27) | NO: 28) | (SEQ ID |
| | | NO: 26) | NO: 26) | NO: 26) | | | NO: 26) |

VH Sequence:
EVQLQQSGPELVKPGASMKISCKASGFSFTGYTMNWVKQSHVKNLEWIGLISPYDGGT
SYNQKFKGKATLTVDKSSSTAYMELLSLTSEDSAVYYCARRAYGYAMDYWGQGTSVTV
SS (SEQ ID NO: 36)
VL Sequence:
QIVLTQSPAIMSASPGEKVTMTCSASSSVSYMYWYQQKPGSSPRLLIYDTSNLASGV
PLRFSGSGSGTSYSLTISRMEAEDAATYYCQQWSSYPFTFGSGTKLEIK
(SEQ ID NO: 30)

Anti-VISTA monoclonal antibodies of the disclosure include both intact molecules, and antibody fragments (such as, for example, Fab and F(ab′)2 fragments) which are capable of specifically binding to VISTA. Fab and F(ab′)2 fragments lack the Fc fragment of intact antibody, clear more rapidly from the circulation of the animal or plant, and may have less non-specific tissue binding than an intact antibody (Wahl et al., 1983, J. Nucl. Med. 24:316). Antibody fragments are therefore useful in therapeutic applications among other applications.

The term “antibody fragment” refers to a portion of a full-length antibody, generally the target binding or variable region. Examples of antibody fragments include Fab, Fab′, F(ab′)2 and Fv fragments. An “Fv” fragment is the minimum antibody fragment which contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target, although at a lower affinity than the entire binding site. “Single-chain Fv” or “scFv” antibody fragments comprise the VH and VL domains of an antibody, wherein these domains are present in a single polypeptide chain. Generally, the Fv polypeptide further comprises a polypeptide linker between the VH and VL domains which enables the scFv to form the desired structure for target binding. “Single domain antibodies” are composed of a single VH or VL domains which exhibit sufficient affinity to VISTA. In a specific embodiment, the single domain antibody is a camelized antibody (See, e.g., Riechmann, 1999, Journal of Immunological Methods 231:25-38).

The Fab fragment contains the constant domain of the light chain and the first constant domain (CH1) of the heavy chain. Fab′ fragments differ from Fab fragments by the addition of a few residues at the carboxyl terminus of the heavy chain CHI domain including one or more cysteines from the antibody hinge region. F(ab′) fragments are produced by cleavage of the disulfide bond at the hinge cysteines of the F(ab′)2 pepsin digestion product. Additional chemical couplings of antibody fragments are known to those of ordinary skill in the art.

The Anti-VISTA monoclonal antibodies of the disclosure can be chimeric antibodies. The term “chimeric” antibody as used herein refers to an antibody having variable sequences derived from a non-human immunoglobulins, such as rat or mouse antibody, and human immunoglobulins constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816397, which are incorporated herein by reference in their entireties.

The Anti-VISTA monoclonal antibodies of the disclosure can be humanized. “Humanized” forms of non-human (e.g., murine) antibodies are chimeric immunoglobulins, immunoglobulin chains or fragments thereof (such as Fv, Fab, Fab′, F(ab′)2 or other target-binding subsequences of antibodies) which contain minimal sequences derived from non-human immunoglobulins. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence, and can be referred to as “CDR-grafted.” The humanized antibody can also comprise at least a portion of an immunoglobulin constant region (Fc), typically that of a human immunoglobulin consensus sequence. Methods of antibody humanization, including methods of designing humanized antibodies, are known in the art. See, e.g., Lefranc et al., 2003, Dev. Comp. Immunol. 27:55-77; Lefranc et al., 2009, Nucl. Acids Res. 37: D1006-1012; Lefranc, 2008, Mol. Biotechnol. 40: 101-111; Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are hereby incorporated by reference in their entireties.

### Polynucleotides Encoding an Antibody

Also provided herein are polynucleotides comprising a nucleotide sequence encoding an antibody provided herein that binds to PSGL-1 (e.g., PSGL-1 polypeptide, PSGL-1 polypeptide fragment, PSGL-1 epitope). Also provided herein are polynucleotides that hybridize under high stringency, intermediate or lower stringency hybridization conditions, e.g., as defined supra, to polynucleotides that encode a antibody or modified antibody provided herein.

Also provided herein are polynucleotides comprising a nucleotide sequence encoding an antibody provided herein that binds to VISTA (e.g., VISTA polypeptide, VISTA polypeptide fragment, VISTA epitope). Also provided herein are polynucleotides that hybridize under high stringency, intermediate or lower stringency hybridization conditions, e.g., as defined supra, to polynucleotides that encode a antibody or modified antibody provided herein.

In certain embodiments, nucleic acid molecules provided herein comprise or consist of a nucleic acid sequence encoding a V_{H} and/or V_{L} amino acid sequence disclosed herein, or any combination thereof (e.g., as a nucleotide sequence encoding an antibody provided herein, such as a full-length antibody, heavy and/or light chain of an antibody, or a single chain antibody provided herein).

### Recombinant Expression of an Antibody

A variety of expression systems may be used to express the present antibodies, e.g., an anti-PSGL-1 antibody or an anti-VISTA antibody as described herein. In one aspect, such expression systems represent vehicles by which the coding sequences of interest may be produced and subsequently purified, but also represent cells which may, when transiently transfected with the appropriate nucleotide coding sequences, express an antibody of the invention in situ.

The invention provides vectors comprising the polynucleotides described herein. In one embodiment, the vector contains a polynucleotide encoding a heavy chain of an IgG antibody of the invention, i.e. an antibody which carries a mutation in the Fc domain. In another embodiment, said polynucleotide encodes the light chain of an IgG antibody of the invention. The invention also provides vectors comprising polynucleotide molecules encoding fusion proteins, modified antibodies, antibody fragments, and probes thereof.

In order to express the heavy and/or light chain of an antibody disclosed herein, such as an anti-PSGL-1 antibody or an anti-VISTA antibody, the polynucleotides encoding said heavy and/or light chains are inserted into expression vectors such that the genes are operatively linked to transcriptional and translational sequences.

“Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing and can also include additional components whose presence is advantageous, for example, leader sequences and fusion partner sequences.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e. g., bacterial vectors having a bacterial origin of replication and episomal mammalian vectors). Other vectors (e. g., non-episomal mammalian vectors) can be integrated into the genome of a host cell upon introduction into the host cell, and thereby are replicated along with the host genome.

Certain vectors are capable of directing the expression of genes to which they are operatively linked. Such vectors are referred to herein as “recombinant expression vectors” (or simply, “expression vectors”). In general, expression vectors of utility in recombinant DNA techniques are in the form of plasmids. In the present specification, “plasmid” and “vector” may be used interchangeably as the plasmid is the most commonly used form of vector. However, the invention is intended to include such forms of expression vectors, such as bacterial plasmids, YACs, cosmids, retrovirus, EBV-derived episomes, and all the other vectors that the skilled man will know to be convenient for ensuring the expression of the heavy and/or light chains of the antibodies of the invention. The skilled man will realize that the polynucleotides encoding the heavy and the light chains can be cloned into different vectors or in the same vector. In a preferred embodiment, said polynucleotides are cloned into two vectors.

Polynucleotides of the invention and vectors comprising these molecules can be used for the transformation of a suitable host cell. The term “host cell”, as used herein, is intended to refer to a cell into which a recombinant expression vector has been introduced in order to express the present antibody (e.g., an anti-PSGL-1 antibody or an anti-VISTA antibody). It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

Transformation can be performed by any known method for introducing polynucleotides into a cell host. Such methods are well known of the man skilled in the art and include dextran-mediated transformation, calcium phosphate precipitation, polybrene-mediated transfection, protoplast fusion, electroporation, encapsulation of the polynucleotide into liposomes, biolistic injection and direct microinjection of DNA into nuclei.

The host cell may be co-transfected with two or more expression vectors, including the vector expressing the protein of the invention. In particular, the other expression vectors may encode enzymes involved in post-translational modifications, such as glycosylation. For example, a host cell can be transfected with a first vector encoding an antibody as described above (e.g., an anti-PSGL-1 antibody or an anti-VISTA antibody), and a second vector encoding a glycosyltransferase polypeptide. Alternatively, the host cell can be transformed with a first vector encoding an antibody (e.g., an anti-PSGL-1 antibody or an anti-VISTA antibody), a second vector encoding a glycosyltransferase, as described above, and a third vector encoding another glycosyltransferase. Mammalian cells are commonly used for the expression of a recombinant therapeutic immunoglobulins, especially for the expression of whole recombinant antibodies. For example, mammalian cells such as HEK293 or CHO cells, in conjunction with a vector, containing the expression signal such as one carrying the major intermediate early gene promoter element from human cytomegalovirus, are an effective system for expressing the present antibody, notably an anti-PSGL-1 antibody or an anti-VISTA antibody (Foecking et al., 1986, Gene 45:101; Cockett et al., 1990, Bio/Technology 8:2).

It is also possible to select a host cell which modulates the expression of the inserted sequences or modifies and processes the gene product in the specific fashion desired. Such modifications (e.g., glycosylation) and processing of protein products may be important for the function of the protein. Different host cells have features and specific mechanisms for the post-translational processing and modification of proteins and gene products. Appropriate cell lines or host systems are chosen to ensure the correct modification and processing of the expressed antibody of interest. Hence, eukaryotic host cells which possess the cellular machinery for proper processing of the primary transcript, glycosylation of the gene product may be used. Such mammalian host cells include, but are not limited to, CHO, COS, HEK293, NS/0, BHK, Y2/0, 3T3 or myeloma cells (all these cell lines are available from public depositeries such as the Collection Nationale des Cultures de Microorganismes, Paris, France, or at the American Type Culture Collection, Manassas, Va., U.S.A.).

For long-term, high-yield production of recombinant proteins, stable expression is preferred. In one embodiment of the invention, cell lines which stably express the antibody (e.g., an anti-PSGL-1 antibody or an anti-VISTA antibody) may be engineered. Rather than using expression vectors which contain viral origins of replication, host cells are transformed with DNA under the control of the appropriate expression regulatory elements, including promoters, enhancers, transcription terminators, polyadenylation sites, and other appropriate sequences known to the person skilled in art, and a selectable marker. Following the introduction of the foreign DNA, engineered cells may be allowed to grow for one to two days in an enriched media, and then are moved to a selective media. The selectable marker on the recombinant plasmid confers resistance to the selection and allows cells to stably integrate the plasmid into a chromosome and be expanded into a cell line. Other methods for constructing stable cell lines are known in the art. In particular, methods for site-specific integration have been developed. According to these methods, the transformed DNA under the control of the appropriate expression regulatory elements, including promoters, enhancers, transcription terminators, polyadenylation sites, and other appropriate sequences is integrated in the host cell genome at a specific target site which has previously been cleaved (Moele et al., Proc. Natl. Acad. Sci. U.S.A., 104(9): 3055-3060; U.S. Pat. Nos. 5,792,632; 5,830,729; 6,238,924; WO 2009/054985; WO 03/025183; WO 2004/067753, all of which are incorporated herein by reference).

A number of selection systems may be used, including but not limited to the Herpes simplex virus thymidine kinase (Wigler et al., Cell 11:223, 1977), hypoxanthine-guanine phosphoribosyltransferase (Szybalska et al., Proc Nati Acad Sci USA 48:202, 1992), glutamate synthase selection in the presence of methionine sulfoximide (Adv Drug Del Rev, 58:671, 2006, and website or literature of Lonza Group Ltd.) and adenine phosphoribosyltransferase (Lowy et al., Cell 22:817, 1980) genes in tk, hgprt or aprt cells, respectively. Also, antimetabolite resistance can be used as the basis of selection for the following genes: dhfr, which confers resistance to methotrexate (Wigler et al., Proc Nati Acad Sci USA 77: 357, 1980); gpt, which confers resistance to mycophenolic acid (Mulligan et al., Proc Nati Acad Sci USA 78: 2072, 1981); neo, which confers resistance to the aminoglycoside, G-418 (Wu et al., Biotherapy 3: 87, 1991); and hygro, which confers resistance to hygromycin (Santerre et al., Gene 30: 147, 1984). Methods known in the art of recombinant DNA technology may be routinely applied to select the desired recombinant clone, and such methods are described, for example, in Ausubel et al., eds., Current Protocols in Molecular Biology, John Wiley Et Sons (1993). The expression levels of an antibody can be increased by vector amplification. When a marker in the vector system expressing an antibody is amplifiable, an increase in the level of inhibitor present in the culture will increase the number of copies of the marker gene. Since the amplified region is associated with the gene encoding the antibody of interest (e.g., an anti-PSGL-1 antibody or an anti-VISTA antibody), production of said antibody will also increase (Crouse et al., Mol Cell Biol 3: 257, 1983). Alternative methods of expressing the gene of the invention exist and are known to the person of skills in the art. For example, a modified zinc finger protein can be engineered that is capable of binding the expression regulatory elements upstream of the gene of the invention; expression of the said engineered zinc finger protein (ZFN) in the host cell of the invention leads to increases in protein production (see e.g., Reik et al., Biotechnol. Bioeng., 97(5), 1180-1189, 2006). Moreover, ZFN can stimulate the integration of a DNA into a predetermined genomic location, resulting in high-efficiency site-specific gene addition (Moehle et al, Proc Nati Acad Sci USA 104:3055, 2007).

The antibody of the invention may be prepared by growing a culture of the transformed host cells under culture conditions necessary to express the desired antibody. The resulting expressed antibody may then be purified from the culture medium or cell extracts. Soluble forms of the antibody can be recovered from the culture supernatant. It may then be purified by any method known in the art for purification of an immunoglobulin molecule, for example, by chromatography (e.g., ion exchange, affinity, particularly by Protein A affinity for Fc, and so on), centrifugation, differential solubility or by any other standard technique for the purification of proteins. Suitable methods of purification will be apparent to a person of ordinary skills in the art.

### Antibody Conjugates and Fusion Proteins

In some embodiments, antibodies provided herein are conjugated or recombinantly fused to a diagnostic, detectable or therapeutic agent or any other molecule. The conjugated or recombinantly fused antibodies can be useful, e.g., for monitoring or prognosing the onset, development, progression and/or severity of a VISTA-mediated disease, disorder or condition as part of a clinical testing procedure, such as determining the efficacy of a particular therapy.

Such diagnosis and detection can accomplished, for example, by coupling the antibody (e.g., an anti-PSGL-1 antibody) to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; chemiluminescent material, such as but not limited to, an acridinium based compound or a HALOTAG; radioactive materials, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Sn; and positron emitting metals using various positron emission tomographies, and non-radioactive paramagnetic metal ions.

Also provided herein are antibodies that are conjugated or recombinantly fused to a therapeutic moiety (or one or more therapeutic moieties), as well as uses thereof. The antibody may be conjugated or recombinantly fused to a therapeutic moiety, such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP), and cisplatin); anthracyclines (e.g., daunorubicin (formerly daunomycin) and doxorubicin); antibiotics (e.g., d actinomycin (formerly actinomycin), bleomycin, mithramycin, and anthramycin (AMC)); Auristatin molecules (e.g., auristatin PHE, auristatin F, monomethyl auristatin E, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN 1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

Further, an antibody provided herein may be conjugated or recombinantly fused to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, γ-interferon, α-interferon, nerve growth factor, platelet derived growth factor, tissue plasminogen activator, an apoptotic agent, e.g., TNF-γ, TNF-γ, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), an anti-angiogenic agent, e.g., angiostatin, endostatin or a component of the coagulation pathway (e.g., tissue factor); or, a biological response modifier such as, for example, a lymphokine (e.g., interferon gamma, interleukin-1 (“IL-1”), interleukin-2 (“IL-2”), interleukin-5 (“IL-5”), interleukin-6 (“IL-6”), interleukin-7 (“IL-7”), interleukin 9 (“IL-9”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid, and fibrin monomer).

Also provided herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90 or about 100 amino acids) to generate fusion proteins, as well as uses thereof. In particular, provided herein are fusion proteins comprising an antigen-binding fragment of an antibody provided herein (e.g., a Fab fragment, Fd fragment, Fv fragment, F(ab)₂ fragment, a VH domain, a VH CDR, a VL domain or a VL CDR) and a heterologous protein, polypeptide, or peptide. In some embodiments, the heterologous protein, polypeptide, or peptide that the antibody is fused to is useful for targeting the antibody to a particular cell type, such as a cell that expresses PSGL-1 or VISTA. For example, an antibody that binds to a cell surface receptor expressed by a particular cell type (e.g., an immune cell) may be fused or conjugated to a modified antibody provided herein.

In addition, an antibody provided herein can be conjugated to therapeutic moieties such as a radioactive metal ion, such as alpha-emitters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In some embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Moreover, antibodies provided herein can be fused to marker sequences, such as a peptide to facilitate purification. In some embodiments, the marker amino acid sequence is a hexa-histidine peptide, such as the tag provided in a pQE vector (QIAGEN, Inc.), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “FLAG” tag.

Methods for fusing or conjugating therapeutic moieties (including polypeptides) to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., 1982, Immunol. Rev. 62:119-58; U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-10539, 1991; Traunecker et al., Nature, 331:84-86, 1988; Zheng et al., J. Immunol., 154: 5590-5600, 1995; Vil et al., Proc. Natl. Acad. Sci. USA, 89:11337-11341, 1992, which are incorporated herein by reference in their entireties.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of antibodies provided herein (e.g., antibodies with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-313 (each of these patents and publications are hereby incorporated by reference in its entirety). Antibodies, or the encoded antibodies, may be altered by being subjected to random mutagenesis by error-prone PCR, random nucleotide insertion or other methods prior to recombination. A polynucleotide encoding an antibody provided herein may be recombined with one or more components, motifs, sections, parts, domains, fragments, etc. of one or more heterologous molecules.

An antibody provided herein can also be conjugated to a second antibody to form an antibody heteroconjugate as described in U.S. Pat. No. 4,676,980, which is incorporated herein by reference in its entirety.

The therapeutic moiety or drug conjugated or recombinantly fused to an antibody provided herein that binds to a PSGL-1 should be chosen to achieve the desired prophylactic or therapeutic effect(s). In some embodiments, the antibody is a modified antibody. A clinician or other medical personnel should consider the following when deciding on which therapeutic moiety or drug to conjugate or recombinantly fuse to an antibody described herein: the nature of the disease, the severity of the disease, and the condition of the subject.

Antibodies provided herein (e.g., an anti-PSGL-1 antibody or an anti-VISTA) may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Pharmaceutical Compositions

Pharmaceutical compositions, including therapeutic formulations, containing one or more of the therapeutic agents provided herein (e.g., an anti-VISTA therapeutic agent, such as an anti-VISTA antibody) can be prepared for storage by mixing the antibody having the desired degree of purity with optional physiologically acceptable carriers, excipients and/or stabilizers (Remington's Pharmaceutical Sciences (1990) Mack Publishing Co., Easton, Pa.), in the form of lyophilized formulations or aqueous solutions. Acceptable carriers, excipients, and/or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The anti-VISTA therapeutic agents provided herein, notably the anti-VISTA antibodies, can also, for example, be formulated in liposomes. Liposomes containing the molecule of interest are prepared by methods known in the art, such as described in Epstein et al. (1985) Proc. Natl. Acad. Sci. USA 82:3688; Hwang et al. (1980) Proc. Natl. Acad. Sci. USA 77:4030; and U.S. Pat. Nos. 4,485,045 and 4,544,545. Liposomes with enhanced circulation time are disclosed in U.S. Pat. No. 5,013,556.

Particularly useful immunoliposomes can be generated by the reverse phase evaporation method with a lipid composition containing phosphatidylcholine, cholesterol and PEG-derivatized phosphatidylethanolamine (PEG-PE). Liposomes are extruded through filters of defined pore size to yield liposomes with the desired diameter. Fab′ fragments of an antibody provided herein can be conjugated to the liposomes as described in Martin et al. (1982) J. Biol. Chem. 257:286-288 via a disulfide interchange reaction. A chemotherapeutic agent (such as Doxorubicin) is optionally contained within the liposome; See Gabizon et al., (1989) J. National Cancer Inst. 81(19):1484.

Formulations, such as those described herein, can also contain more than one active compound as necessary for the particular indication being treated. In some embodiments, formulations comprise an anti-VISTA therapeutic agent (e.g., an anti-VISTA antibody) provided herein and one or more active compounds with complementary activities that do not adversely affect each other. Such molecules are suitably present in combination in amounts that are effective for the purpose intended. For example, an antibody provided herein can be combined with one or more other therapeutic agents. Such combined therapy can be administered to the patient serially or simultaneously or in sequence.

An anti-VISTA therapeutic agent provided herein (e.g., an anti-VISTA antibody) can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1990) Mack Publishing Co., Easton, Pa.

The formulations to be used for in vivo administration can be sterile. This is readily accomplished by filtration through, e.g., sterile filtration membranes.

Sustained-release preparations can also be prepared. Suitable examples of sustained-release preparations include semipermeable matrices of solid hydrophobic polymers containing the polypeptide, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

In some embodiments, the pharmaceutical compositions provided herein contain therapeutically effective amounts of one or more of the anti-VISTA therapeutic agents provided herein (e.g., an anti-VISTA antibody), and optionally one or more additional prophylactic of therapeutic agents, in a pharmaceutically acceptable carrier. Such pharmaceutical compositions are useful in the prevention, treatment, or alleviation of one or more symptoms of a VISTA-mediated disease, disorder or condition.

Pharmaceutical carriers suitable for administration of the compounds provided herein include any such carriers known to those skilled in the art to be suitable for the particular mode of administration.

In addition, the anti-VISTA therapeutic agents provided herein, notably the anti-VISTA antibodies, may be formulated as the sole pharmaceutically active ingredient in the composition or may be combined with other active ingredients (such as one or more other prophylactic or therapeutic agents).

The compositions can contain one or more antibodies provided herein. In some embodiments, the anti-VISTA therapeutic agents provided herein (e.g., anti-VISTA antibodies) are formulated into suitable pharmaceutical preparations, such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In some embodiments, the anti-VISTA therapeutic agents provided herein (e.g., the anti-VISTA antibodies) described above are formulated into pharmaceutical compositions using techniques and procedures well known in the art (see, e.g., Ansel (1985) Introduction to Pharmaceutical Dosage Forms, 4^{th} Ed., p. 126).

In some embodiments of the compositions, effective concentrations of one or more anti-VISTA therapeutic agents (e.g., an anti-VISTA antibody) is (are) mixed with a suitable pharmaceutical carrier. In some embodiments, concentrations of the compounds in the compositions are effective for delivery of an amount, upon administration, that treats, prevents, or ameliorates a VISTA-mediated disease, disorder or condition, or symptom thereof.

In some embodiments, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

In some embodiments, the anti-VISTA therapeutic agent provided herein (e.g., an anti-VISTA antibody) is included in the pharmaceutically acceptable carrier in an effective amount sufficient to exert a therapeutically useful effect in the absence of undesirable side effects on the patient treated. The therapeutically effective concentration can be determined empirically by testing the compounds in in vitro and in vivo systems using routine methods and then extrapolated therefrom for dosages for humans.

The concentration of the anti-VISTA therapeutic agent (e.g., an anti-VISTA antibody) in the pharmaceutical composition will depend on, e.g., the physicochemical characteristics of the therapeutic agent, the dosage schedule, and amount administered as well as other factors known to those of skill in the art.

In some embodiments, a therapeutically effective dosage produces a serum concentration of anti-VISTA therapeutic agent (e.g., an anti-VISTA antibody) of from about 0.1 ng/ml to about 50-100 μg/ml. The pharmaceutical compositions, in another embodiment, provide a dosage of from about 0.001 mg to about 2000 mg of therapeutic agent (e.g., of antibody) per kilogram of body weight per day. Pharmaceutical dosage unit forms can be prepared to provide from about 0.01 mg, 0.1 mg or 1 mg to about 500 mg, 1000 mg or 2000 mg, and in some embodiments from about 10 mg to about 500 mg of the anti-VISTA therapeutic agent (e.g., an anti-VISTA antibody) and/or a combination of other optional essential ingredients per dosage unit form.

The anti-VISTA therapeutic agent (e.g., an anti-VISTA antibody) can be administered at once, or may be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens can be adjusted over time according to the individual need and the professional judgment of the person administering or supervising the administration of the compositions, and that the concentration ranges set forth herein are exemplary only and are not intended to limit the scope or practice of the claimed compositions.

Upon mixing or addition of the anti-VISTA therapeutic agent, the resulting mixture can be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

In some embodiments, the pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The anti-VISTA therapeutic agent (e.g., an anti-VISTA antibody) is, in some embodiments, formulated and administered in unit-dosage forms or multiple-dosage forms. “Unit-dose” forms as used herein refers to physically discrete units suitable for human and animal subjects and packaged individually as is known in the art. Each unit-dose contains a predetermined quantity of the therapeutic agent sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A “multiple-dose” form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

In some embodiments, one or more anti-VISTA therapeutic agents (e.g., an anti-VISTA antibody) provided herein are in a liquid pharmaceutical formulation. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences (1990) Mack Publishing Co., Easton, Pa.

Dosage forms or compositions containing a therapeutic agent, specifically an antibody, in the range of 0.005% to 100% with the balance made up from non-toxic carrier can be prepared. Methods for preparation of these compositions are known to those skilled in the art.

Oral pharmaceutical dosage forms are either solid, gel or liquid. The solid dosage forms include tablets, capsules, granules, and bulk powders. Types of oral tablets include compressed, chewable lozenges and tablets which may be enteric-coated, sugar-coated or film-coated. Capsules can be hard or soft gelatin capsules, while granules and powders can be provided in non-effervescent or effervescent form with the combination of other ingredients known to those skilled in the art.

In some embodiments, the formulations are solid dosage forms. In some embodiments, the formulations are capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water-soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The anti-VISTA therapeutic agents (e.g., anti-VISTA antibodies) provided herein can be provided in a composition that protects it from the acidic environment of the stomach. For example, the composition can be formulated in an enteric coating that maintains its integrity in the stomach and releases the active compound in the intestine. The composition can also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The therapeutic agent can also be mixed with other active materials which do not impair the desired action, or with materials that supplement the desired action, such as antacids, H2 blockers, and diuretics. The active ingredient is an anti-VISTA therapeutic agent, notably an antibody, or pharmaceutically acceptable derivative thereof as described herein. Higher concentrations, up to about 98% by weight of the active ingredient may be included.

In some embodiments, tablets and capsules formulations can be coated as known by those of skill in the art in order to modify or sustain dissolution of the active ingredient. Thus, for example, they may be coated with a conventional enterically digestible coating, such as phenylsalicylate, waxes and cellulose acetate phthalate.

In some embodiments, the formulations are liquid dosage forms. Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is, in some embodiments, encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, can be diluted with a sufficient quantity of a pharmaceutically acceptable liquid carrier, e.g., water, to be easily measured for administration.

Alternatively, liquid or semi-solid oral formulations can be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

Parenteral administration, in some embodiments, is characterized by injection, either subcutaneously, intramuscularly, intratumorally, or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The therapeutic agent (e.g., an antibody) diffuses through the outer polymeric membrane in a release rate controlling step. The amount of therapeutic agent contained in such parenteral compositions is highly dependent on the specific nature thereof, as well as the activity of the compound and the needs of the subject.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active anti-VISTA therapeutic agent (e.g., an anti-VISTA antibody) is adjusted so that an injection provides an effective amount to produce the desired pharmacological effect. The exact dose depends on the age, weight and condition of the patient or animal as is known in the art.

The unit-dose parenteral preparations can be packaged in an ampoule, a vial or a syringe with a needle. All preparations for parenteral administration can be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In some embodiments, a therapeutically effective dosage is formulated to contain a concentration of at least about 0.1% w/w up to about 90% w/w or more, in some embodiments more than 1% w/w of the active compound to the treated tissue(s).

The therapeutic agent, such as an antibody, can be suspended in micronized or other suitable form. The form of the resulting mixture depends upon a number of factors, including the intended mode of administration and the solubility of the compound in the selected carrier or vehicle. The effective concentration is sufficient for ameliorating the symptoms of a VISTA-mediated disease, disorder or condition, and may be empirically determined.

In some embodiments, the pharmaceutical formulations are lyophilized powders, which can be reconstituted for administration as solutions, emulsions and other mixtuRes. They may also be reconstituted and formulated as solids or gels.

The lyophilized powder is prepared by dissolving a therapeutic agent, such as an antibody, provided herein, or a pharmaceutically acceptable derivative thereof, in a suitable solvent. In some embodiments, the lyophilized powder is sterile. The solvent may contain an excipient which improves the stability or other pharmacological component of the powder or reconstituted solution, prepared from the powder.

Excipients that may be used include, but are not limited to, dextrose, sorbitol, fructose, corn syrup, xylitol, glycerin, glucose, sucrose or any other suitable agent. The solvent may also contain a buffer, such as citrate, sodium or potassium phosphate or other such buffer known to those of skill in the art at, in some embodiments, about neutral pH. Subsequent sterile filtration of the solution followed by lyophilization under standard conditions known to those of skill in the art provides the desired formulation. In some embodiments, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or any other suitable carrier. The precise amount depends upon the selected compound. Such amount can be empirically determined.

Topical mixtures are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The therapeutic agents provided herein can be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in some embodiments, have diameters of less than 50 microns, in some embodiments less than 10 microns.

The therapeutic agents can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

Pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in some embodiments, is about 2 to 3 gm.

Tablets and capsules for rectal administration can be manufactured using the same pharmaceutically acceptable substance and by the same methods as for formulations for oral administration.

The therapeutic agents (for example, antibodies) and other compositions provided herein may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some embodiment, liposomal suspensions, including tissue-targeted liposomes, such as tumor-targeted liposomes, may also be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

Methods of Treatment, Prevention and/or Alleviation

In another aspect, the present invention also relates to an anti-VISTA therapeutic agent (e.g., an anti-VISTA antibody) for use in the treatment of a VISTA-mediated disease disorder or condition in a patient. Provided herein is an anti-VISTA-therapy (e.g., an anti-VISTA antibody) provided herein for use in the prevention, treatment and/or alleviation of one or more symptoms of a disease, disorder or condition, such as a VISTA-mediated disease, disorder or condition, notably a VISTA-mediated cancer. Advantageously, said VISTA-mediated disease, disorder or condition has been previously been detected or diagnosed by one of the methods provided herein.

In an embodiment, the present invention relates to an anti-VISTA therapeutic agent (e.g., an anti-VISTA antibody) for use in the treatment of a VISTA-mediated disease, disorder, or condition in a patient, wherein said VISTA-mediated disease, disorder or condition has been previously been detected or diagnosed by one of the methods provided herein. In other words, the invention thus relates to an anti-VISTA therapeutic agent to treat a VISTA-mediated disease, disorder, or condition in a patient, wherein the Anti-VISTA therapeutic agent is administered to a patient who has been diagnosed with a VISTA-mediated disease, disorder, or condition using a method described above.

In some embodiments, provided herein are compositions comprising one or more antibodies (e.g., an anti-VISTA antibody) provided herein for use in the management, prevention, or treatment of a VISTA-mediated disease, disorder or condition and/or the alleviation of one or more symptoms of a VISTA-mediated disease, disorder or condition. Exemplary VISTA-mediated diseases, disorders or conditions include a cell proliferative disorder, tumor, and graft-versus-host disease (GVHD), or a symptom thereof. Preferably, said VISTA-mediated disease, disorder or condition is a cancer.

It is thus herein provided an anti-VISTA therapeutic agent (e.g., an anti-VISTA antibody) for use in the treatment of a VISTA-mediated cancer in a patient, said use comprising:
a) contacting a biological sample of said subject with a reagent capable of binding specifically to PSGL-1 nucleic acid or protein; and
b) quantifying the binding of said reagent with said biological sample, thus determining the level of expression of PSGL-1 in said sample.

According to a preferred embodiment, the present use further comprises a step of scoring the tumor by comparing the level of expression of PSGL-1 in the biological sample of the subject (e.g. by immune infiltrates of a tumor microenvironment) to an appropriate scale based on two parameters which are the intensity of staining and the percentage of positive cells.

In another embodiment, the present invention relates to an anti-VISTA therapeutic agent (e.g., an anti-VISTA antibody) for use in the treatment of a VISTA-mediated cancer in a patient, wherein said use comprises the prior determination of the PSGL-1 status of said tumor, as described above. According to this embodiment, a tumor which is [PSGL-1 (+)] is indicative of a VISTA-mediated cancer and is thus susceptible to respond to treatment with an anti-VISTA therapeutic agent (e.g., an anti-VISTA antibody).

According to another preferred embodiment, the present use further comprises comparing the level of expression of PSGL-1 in the biological sample of the subject (e.g. by immune infiltrates of a tumor microenvironment) with a reference level.

According to this preferred embodiment, the anti-VISTA therapeutic agent (e.g., an anti-VISTA antibody) is for use in the treatment of a VISTA-mediated cancer in a patient, said use comprising:
a) determining the level of expression of PSGL-1 in a biological sample of said subject, e.g., by immune infiltrates of a tumor microenvironment in said biological sample;
b) comparing the level of expression of step a) with a reference level; and
c) determining a VISTA-mediated cancer when the level of expression of step a) is higher than the reference level.

According to another preferred embodiment, the anti-VISTA therapeutic agent (e.g., an anti-VISTA antibody) is for use in the treatment of a VISTA-mediated cancer in a patient, said use comprising:
a) determining the level of expression of PSGL-1 in a biological sample of said subject, e.g., by immune infiltrates of a tumor microenvironment in said biological sample;
b) comparing the level of expression of step a) with a reference level; and
c) diagnosing a VISTA-mediated cancer when the level of expression of step a) is higher than the reference level.

Advantageously, the method of the invention comprises both steps of:
scoring the tumor by comparing the level of expression of PSGL-1 in the biological sample of the subject (e.g. by immune infiltrates of a tumor microenvironment) to an appropriate scale based on two parameters which are the intensity of staining and the percentage of positive cells; and
comparing the level of expression of PSGL-1 in the biological sample of the subject (e.g. by immune infiltrates of a tumor microenvironment) with a reference level.

Advantageously, the above use of the anti-VISTA therapeutic agent will further comprise determining the level of expression of at least one of VISTA, CD11b, CD33, CD4, and CD8, as detailed above. In such cases, a level of expression of PSGL-1 and at least one of VISTA, CD11b, CD33, CD4, and CD8, or the relative expression levels thereof, higher than the reference level indicates a VISTA-mediated cancer.

According to another embodiment, the invention is drawn to an anti-VISTA therapeutic agent (e.g., an anti-VISTA antibody) for use in the treatment of a VISTA-mediated cancer in a patient, said use comprising:
a) contacting a biological sample of said subject with a reagent capable of binding specifically to PSGL-1 nucleic acid or protein; and
b) quantifying the binding of said reagent with said biological sample, thus determining the level of expression of PSGL-1 in said sample; and
c) adapting the treatment of the anti-VISTA therapeutic agent based on the level of step a).

According to a preferred embodiment, the present use further comprises a step of scoring the tumor by comparing the level of expression of PSGL-1 in the biological sample of the subject (e.g. by immune infiltrates of a tumor microenvironment) to an appropriate scale based on two parameters which are the intensity of staining and the percentage of positive cells.

In another embodiment, the present invention relates to an anti-VISTA therapeutic agent (e.g., an anti-VISTA antibody) for use in the treatment of a VISTA-mediated cancer in a patient, wherein said use comprises the prior determination of the PSGL-1 status of said tumor, as described above. According to this embodiment, a tumor which is [PSGL-1 (+)] is indicative of a VISTA-mediated cancer and is thus susceptible to respond to treatment with an anti-VISTA therapeutic agent (e.g., an anti-VISTA antibody).

According to another preferred embodiment, the present use further comprises comparing the level of expression of PSGL-1 in the biological sample of the subject (e.g. by immune infiltrates of a tumor microenvironment) with a reference level.

Said adaptation of the anti-VISTA therapeutic agent treatment may consist in:
a reduction or suppression of the said anti-VISTA therapeutic agent treatment if the patient has been diagnosed as non-responding to the anti-VISTA therapeutic agent, or
the continuation of the said anti-VISTA therapeutic agent treatment if the patient has been diagnosed as responding to the anti-VISTA therapeutic agent.

A patient is responding to said treatment if there is a difference of PSGL-1 expression between the expression level of step a) and the reference level. For example, a difference of PSGL-1 expression between the expression level of step a) and the expression level of PSGL-1 in a second biological sample from the patient obtained prior to being treated indicates whether said patient is responding to said treatment. Advantageously, a higher level of PSGL-1 expression in step a) compared to the expression level of PSGL-1 in a second biological sample from the patient obtained prior to being treated indicates that said patient is responding to said treatment.

In some embodiments, the above use comprises the determining the level of expression of at least one of VISTA, CD11b, CD33, CD4, and CD8, in addition to PSGL-1 and comparing the level of expression of PSGL-1 and at least one of VISTA, CD11b, CD33, CD4, and CD8, or the relative expression levels thereof, in the first biological sample with the level of expression of PSGL-1 and at least one of VISTA, CD11 b, CD33, CD4, and CD8, or the relative expression levels thereof, in a second biological sample from the patient obtained prior to being treated. In this case, a differential level of expression or relative expression levels of PSGL-1 and at least one of VISTA, CD11b, CD33, CD4, and CD8 in the first biological sample compared to the level of expression or relative expression levels of PSGL-1 and at least one of VISTA, CD11b, CD33, CD4, and CD8 in the second biological sample indicates that the patient is responding to treatment.

In some aspects of this method, the treatment includes administering an anti-VISTA antibody and/or an anti-PSGL-1 antibody as described herein.

In some aspects, the method includes wherein the first biological sample comprises immune infiltrates of a tumor microenvironment.

In some embodiment, provided herein are methods for preventing or treating a disease, disorder or condition described herein by administering to a subject a therapeutically effective amount of an anti-VISTA therapeutic agent (e.g., an anti-VISTA antibody), including as described herein, or a composition thereof. In some embodiments, a method for treating the disease, disorder or condition comprises administering to subject a therapeutically effective amount of a pharmaceutical composition comprising an anti-VISTA antibody and a pharmaceutically acceptable carrier, excipient and/or stabilizer. A method provided herein can also optionally include at least one additional therapeutic agent, such as those described herein (e.g., an anti-VISTA antibody), either as a separate treatment or in combination. Also described herein are compositions, including pharmaceutical compositions, comprising an anti-VISTA therapeutic agent (e.g., an anti-VISTA antibody) provided herein for use in the treatment, prevention, and/or alleviation of one or more symptom of a disease, disorder or condition, such as a VISTA-mediated disease, disorder or condition. An exemplary VISTA-mediated disease, disorder or condition includes a cell proliferative disorder (e.g., cancer or tumor) or a symptom thereof.

In some embodiments, described herein are compositions comprising an anti-VISTA therapeutic agent (e.g., an anti-VISTA antibody) for use in the prevention, treatment and/or alleviation of one or more symptoms of a VISTA-mediated disease, disorder, or condition such as a cell proliferative disorder. A cell proliferative disorder includes cancer or tumor formation, or a symptom thereof. In some embodiments, the cell proliferative disorder is associated with increased expression and/or activity of VISTA. In some embodiments, the cell proliferative disorder is associated with increased expression of VISTA on the surface of a cancer cell.

In another aspect, the present invention also relates to an anti-VISTA therapeutic agent (e.g., an anti-VISTA antibody) for use in the treatment of a PSGL-1-mediated disease disorder or condition in a patient. Provided herein is an anti-VISTA-therapy (e.g., an anti-VISTA antibody) provided herein for use in the prevention, treatment and/or alleviation of one or more symptoms of a disease, disorder or condition, such as a PSGL-1-mediated disease, disorder or condition, notably a PSGL-1-mediated cancer. Advantageously, said PSGL-1-mediated disease, disorder or condition has been previously been detected or diagnosed by one of the methods provided herein.

In an embodiment, the present invention relates to an anti-VISTA therapeutic agent (e.g., an anti-VISTA antibody) for use in the treatment of a PSGL-1-mediated disease, disorder, or condition in a patient, wherein said PSGL-1-mediated disease, disorder or condition has been previously been detected or diagnosed by one of the methods provided herein. In other words, the invention thus relates to an anti-VISTA therapeutic agent to treat a PSGL-1-mediated disease, disorder, or condition in a patient, wherein the Anti-VISTA therapeutic agent is administered to a patient who has been diagnosed with a PSGL-1-mediated disease, disorder, or condition using a method described above.

In some embodiments, provided herein are compositions comprising one or more antibodies (e.g., an anti-VISTA antibody) provided herein for use in the management, prevention, or treatment of a PSGL-1-mediated disease, disorder or condition and/or the alleviation of one or more symptoms of a PSGL-1-mediated disease, disorder or condition. Exemplary PSGL-1-mediated diseases, disorders or conditions include a cell proliferative disorder, tumor, and graft-versus-host disease (GVHD), ora symptom thereof. Preferably, said PSGL-1-mediated disease, disorder or condition is a cancer.

It is thus herein provided an anti-VISTA therapeutic agent (e.g., an anti-VISTA antibody) for use in the treatment of a PSGL-1-mediated cancer in a patient, said use comprising:
c) contacting a biological sample of said subject with a reagent capable of binding specifically to PSGL-1 nucleic acid or protein; and
d) quantifying the binding of said reagent with said biological sample, thus determining the level of expression of PSGL-1 in said sample.

According to a preferred embodiment, the present use further comprises a step of scoring the tumor by comparing the level of expression of PSGL-1 in the biological sample of the subject (e.g. by immune infiltrates of a tumor microenvironment) to an appropriate scale based on two parameters which are the intensity of staining and the percentage of positive cells.

In another embodiment, the present invention relates to an anti-VISTA therapeutic agent (e.g., an anti-VISTA antibody) for use in the treatment of a PSGL-1-mediated cancer in a patient, wherein said use comprises the prior determination of the PSGL-1 status of said tumor, as described above. According to this embodiment, a tumor which is [PSGL-1 (+)] is indicative of a PSGL-1-mediated cancer and is thus susceptible to respond to treatment with an anti-VISTA therapeutic agent (e.g., an anti-VISTA antibody).

According to another preferred embodiment, the present use further comprises comparing the level of expression of PSGL-1 in the biological sample of the subject (e.g. by immune infiltrates of a tumor microenvironment) with a reference level.

According to this preferred embodiment, the anti-VISTA therapeutic agent (e.g., an anti-VISTA antibody) is for use in the treatment of a PSGL-1-mediated cancer in a patient, said use comprising:
d) determining the level of expression of PSGL-1 in a biological sample of said subject, e.g., by immune infiltrates of a tumor microenvironment in said biological sample;
e) comparing the level of expression of step a) with a reference level; and
f) determining a PSGL-1-mediated cancer when the level of expression of step a) is higher than the reference level.

According to another preferred embodiment, the anti-VISTA therapeutic agent (e.g., an anti-VISTA antibody) is for use in the treatment of a PSGL-1-mediated cancer in a patient, said use comprising:
d) determining the level of expression of PSGL-1 in a biological sample of said subject, e.g., by immune infiltrates of a tumor microenvironment in said biological sample;
e) comparing the level of expression of step a) with a reference level; and
f) diagnosing a PSGL-1-mediated cancer when the level of expression of step a) is higher than the reference level.

Advantageously, the method of the invention comprises both steps of:
scoring the tumor by comparing the level of expression of PSGL-1 in the biological sample of the subject (e.g. by immune infiltrates of a tumor microenvironment) to an appropriate scale based on two parameters which are the intensity of staining and the percentage of positive cells; and
comparing the level of expression of PSGL-1 in the biological sample of the subject (e.g. by immune infiltrates of a tumor microenvironment) with a reference level.

Advantageously, the above use of the anti-VISTA therapeutic agent will further comprise determining the level of expression of at least one of VISTA, CD11b, CD33, CD4, and CD8, as detailed above. In such cases, a level of expression of PSGL-1 and at least one of VISTA, CD11b, CD33, CD4, and CD8, or the relative expression levels thereof, higher than the reference level indicates a PSGL-1-mediated cancer.

According to another embodiment, the invention is drawn to an anti-VISTA therapeutic agent (e.g., an anti-VISTA antibody) for use in the treatment of a PSGL mediated cancer in a patient, said use comprising:
d) contacting a biological sample of said subject with a reagent capable of binding specifically to PSGL-1 nucleic acid or protein; and
e) quantifying the binding of said reagent with said biological sample, thus determining the level of expression of PSGL-1 in said sample; and
f) adapting the treatment of the anti-VISTA therapeutic agent based on the level of step a).

According to a preferred embodiment, the present use further comprises a step of scoring the tumor by comparing the level of expression of PSGL-1 in the biological sample of the subject (e.g. by immune infiltrates of a tumor microenvironment) to an appropriate scale based on two parameters which are the intensity of staining and the percentage of positive cells.

In another embodiment, the present invention relates to an anti-VISTA therapeutic agent (e.g., an anti-VISTA antibody) for use in the treatment of a PSGL-1-mediated cancer in a patient, wherein said use comprises the prior determination of the PSGL-1 status of said tumor, as described above. According to this embodiment, a tumor which is [PSGL-1 (+)] is indicative of a PSGL-1-mediated cancer and is thus susceptible to respond to treatment with an anti-VISTA therapeutic agent (e.g., an anti-VISTA antibody).

According to another preferred embodiment, the present use further comprises comparing the level of expression of PSGL-1 in the biological sample of the subject (e.g. by immune infiltrates of a tumor microenvironment) with a reference level.

Said adaptation of the anti-VISTA therapeutic agent treatment may consist in:
a reduction or suppression of the said anti-VISTA therapeutic agent treatment if the patient has been diagnosed as non-responding to the anti-VISTA therapeutic agent, or
the continuation of the said anti-VISTA therapeutic agent treatment if the patient has been diagnosed as responding to the anti-VISTA therapeutic agent.

A patient is responding to said treatment if there is a difference of PSGL-1 expression between the expression level of step a) and the reference level. For example, a difference of PSGL-1 expression between the expression level of step a) and the expression level of PSGL-1 in a second biological sample from the patient obtained prior to being treated indicates whether said patient is responding to said treatment. Advantageously, a higher level of PSGL-1 expression in step a) compared to the expression level of PSGL-1 in a second biological sample from the patient obtained prior to being treated indicates that said patient is responding to said treatment.

Examples of cell proliferative disorders to be treated, prevented, or symptoms of which can be alleviated by the antibodies provided herein include, but are not limited to, hematological cancers (e.g., leukemias, lymphomas, or myelomas), bladder, breast, colon, connective tissue, rectal, gastric, esophageal, lung, larynx, kidney, oral, ovarian, or prostate cancers, or sarcomas, melanomas, or gliomas, or metastases of any of these cancers. Exemplary hematological cancers include, but are not limited to, acute myeloid leukemia (AML), acute lymphoblastic leukemia (ALL), chronic myelogenous leukemia (CML), chronic lymphocytic leukemia (CLL), acute monocytic leukemia (AMoL), Hodgkin lymphoma, non-Hodgkin lymphoma, multiple myeloma, plasmacytoma, localized myeloma or extramedullary myeloma.

In some embodiments, the hematological cancer is a lymphoma. In other embodiments, the hematological cancer is a leukemia. In some embodiments, the hematological cancer is a myeloma. In another embodiment, the hematological cancer is acute myeloid leukemia (AML). In another embodiment, the hematological cancer is acute lymphoblastic leukemia (ALL). In another embodiment, the hematological cancer is chronic myelogenous leukemia (CML). In another embodiment, the hematological cancer is chronic lymphocytic leukemia (CLL). In another embodiment, the hematological cancer is acute monocytic leukemia (AMoL). In another embodiment, the hematological cancer is Hodgkin lymphoma. In another embodiment, the hematological cancer is a non-Hodgkin lymphoma. In another embodiment, the hematological cancer is multiple myeloma. In another embodiment, the hematological cancer is plasmacytoma. In another embodiment, the hematological cancer is localized myeloma. In another embodiment, the hematological cancer is extramedullary myeloma.

In some embodiments, the hematological cancer is myelodysplastic syndrome, an acute leukemia, e.g., acute T cell leukemia, acute myelogenous leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), or acute biphenotypic leukemia; a chronic leukemia, e.g., chronic myeloid lymphoma, chronic myelogenous leukemia (CML), chronic monocytic leukemia, small lymphocytic lymphoma, or B-cell prolymphocytic leukemia; hairy cell lymphoma; T cell prolymphocytic leukemia; or a lymphoma, e.g., histiocytic lymphoma, lymphoplasmacytic lymphoma (e.g., Waldenström macroglobulinemia), splenic marginal zone lymphoma, plasma cell neoplasm (e.g., plasma cell myeloma, plasmacytoma, a monoclonal immunoglobulin deposition disease, or a heavy chain disease), extranodal marginal zone B cell lymphoma (MALT lymphoma), nodal marginal zone B cell lymphoma (NMZL), follicular lymphoma, mantle cell lymphoma, diffuse large B cell lymphoma, mediastinal (thymic) large B cell lymphoma, intravascular large B cell lymphoma, primary effusion lymphoma, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides (Sezary syndrome), a primary cutaneous CD30-positive T cell lymphoproliferative disorder (e.g., primary cutaneous anaplastic large cell lymphoma or lymphomatoid papulosis), angioimmunoblastic T cell lymphoma, peripheral T cell lymphoma, unspecified, anaplastic large cell lymphoma, a Hodgkin's lymphoma or a nodular lymphocyte-predominant Hodgkin's lymphoma.

An anti-VISTA therapeutic agent (e.g., an anti-VISTA antibody) described herein can be administered to a human for therapeutic purposes. Moreover, an anti-VISTA therapeutic agent (e.g., an anti-VISTA antibody) can be administered to a non-human mammal expressing VISTA with which the antibody cross-reacts (e.g., a primate, pig, rat, or mouse) for veterinary purposes or as an animal model of human disease. Regarding the latter, such animal models may be useful for evaluating the therapeutic efficacy of antibodies provided herein (e.g., testing of dosages and time courses of administration).

In some embodiments, the anti-VISTA therapeutic agent is an antibody which can be used in a method of modulating T cell function mediated by binding of VISTA to PSGL-1. Such methods can include contacting the T cell with an anti-VISTA antibody described herein. In some embodiments, the anti-PSGL-1 antibody does not block or inhibit the binding of PSGL-1 to P-selectin, L-selectin or E-selectin. In some embodiments, the method for modulating T cell function includes administering an effective amount of a composition comprising an anti-VISTA antibody provided herein to a subject. In some aspects, the T cell function that is modulated includes increasing T cell activation. Such T cell activation can further include increasing T cell proliferation. Methods for assaying the modulation of an immune response are well known to one of skill in the art, and it is understood that a skilled artisan would be able to readily conduct such assays.

In some embodiments, an anti-VISTA therapeutic agent (e.g., an anti-VISTA antibody) or a composition comprising an anti-VISTA therapeutic agent (e.g., an anti-VISTA antibody), including as described herein, can be used either alone or in combination with another compound or treatment. For example, in some embodiments, the other compound is an antagonist to a co-inhibitory molecule or an agonist to a co-stimulatory molecule. In such embodiments, the combined therapy leads to reinvigoration or de novo activation of the immune system through activated T cells that is greater than the administration of either compound or treatment individually. This activation of the immune system will result in a highly beneficial physiological response in the treatment of a VISTA mediated disease, disorder or condition, including in the context of cancer treatment (e.g., hematological cancer treatment).

In some embodiments, the methods described herein can include administering a therapeutically effective amount of an anti-VISTA antibody in combination with a therapeutically effective amount of an antagonist to a co-inhibitory molecule. In some embodiments, the co-inhibitory molecule is selected from the group consisting of CD86, CD80, PDL-1, PDL-2, CTLA-4, PD1, LAG3, BTNL2, B7-H3, B7-H4, a butyrophilin, CD48, CD244, TIM-3, CD200R, CD200, CD160, BTLA, HVEM, LAIR1, TIM1, Galectin 9, TIM3, CD48, 2B4, CD155, CD112, CD113 and TIGIT. The antagonist to the co-inhibitory molecule includes an antibody against the co-inhibitory molecule. It is recognized that antagonist to other co-inhibitory molecules are well known in the art, such as those described in Mercier et al., Frontiers in Immunology, 6:418 (2015), Kyi et al., FEBS Letters, 588:368-376 (2014) and Pardoll, Nature Reviews, 12:252-264 (2012). According to this embodiment, the invention relates to the use of an anti-VISTA therapeutic agent (e.g., an anti-VISTA antibody) for use in treatment of VISTA-mediated tumor as described above, said use further comprising the administration of an antagonist to a co-inhibitory molecule, wherein said co-inhibitory molecule is selected from the group consisting of CD86, CD80, PDL-1, PDL-2, CTLA-4, PD1, LAG3, BTNL2, B7-H3, B7-H4, a butyrophilin, CD48, CD244, TIM-3, CD200R, CD200, CD160, BTLA, HVEM, LAIR1, TIM1, Galectin 9, TIM3, CD48, 2B4, CD155, CD112, CD113 and TIGIT.

In some embodiments, the methods described herein can include administering a therapeutically effective amount of an anti-VISTA antibody in combination with a therapeutically effective amount of an agonist to a co-stimulatory molecule. In some embodiments, the co-stimulatory molecule is selected from the group consisting of CD154, TNFRSF25, GITR, 4-1BB, OX40, CD27, TMIGD2, ICOS, CD28, CD40, TL1A, GITRL, 41BBL, OX40L, CD70, HHLA2, ICOSL, a cytokine, LIGHT, HVEM, CD30, CD30L, B7-H2, CD80, CD86, CD40L, TIM4, TIM1, SLAM, CD48, CD58, CD155, CD112, DR3, GITR, CD2, and CD226. The agonist to the co-stimulatory molecule includes an agonistic antibody against the co-stimulatory molecule. It is recognized that agonists to co-stimulatory molecules are well known in the art, such as those described in Mercier et al., Frontiers in Immunology, 6:418 (2015), Kyi et al., FEBS Letters, 588:368-376 (2014) and Capece et al., J. Biomed. Biotechnol. 2012:926321, 17 pages (2012). According to this embodiment, the invention relates to the use of an anti-VISTA therapeutic agent (e.g., an anti-VISTA antibody) for use in treatment of VISTA-mediated tumor as described above, said use further comprising the administration of an agonist to a co-stimulatory molecule, wherein said co-stimulatory molecule is selected from the group consisting of CD154, TNFRSF25, GITR, 4-1BB, OX40, CD27, TMIGD2, ICOS, CD28, CD40, TL1A, GITRL, 41BBL, OX40L, CD70, HHLA2, ICOSL, a cytokine, LIGHT, HVEM, CD30, CD30L, B7-H2, CD80, CD86, CD40L, TIM4, TIM1, SLAM, CD48, CD58, CD155, CD112, DR3, GITR, CD2, and CD226.

In some embodiments, the methods described herein can include administering a therapeutically effective amount of an anti-VISTA therapeutic agent (e.g., an anti-VISTA antibody) in combination with a conventional form of therapy for the treatment of cancer, such as a therapeutically effective amount of a chemotherapeutic agent described herein or a radiation therapy described herein. According to this embodiment, the invention relates to the use of an anti-VISTA therapeutic agent (e.g., an anti-VISTA antibody) for use in treatment of VISTA-mediated tumor as described above, said use further comprising the administration of a conventional form of therapy for the treatment of cancer, such as a therapeutically effective amount of a chemotherapeutic agent described herein or a radiation therapy described herein.

Various delivery systems are known and can be used to administer an anti-VISTA therapeutic agent (e.g., an anti-VISTA antibody as described herein), including, but not limited to, encapsulation in liposomes, microparticles, microcapsules, recombinant cells capable of expressing the antibody, receptor-mediated endocytosis (see, e.g., Wu and Wu, J. Biol. Chem. 262:4429-4432 (1987)), construction of a nucleic acid as part of a retroviral or other vector, etc. Methods of administering a therapeutic agent (e.g., an anti-VISTA antibody provided herein), or pharmaceutical composition include, but are not limited to, parenteral administration (e.g., intradermal, intramuscular, intraperitoneal, intravenous, intratumoral, and subcutaneous), epidural, and mucosal (e.g., intranasal and oral routes). In some embodiments, a therapeutic agent (e.g., an anti-VISTA antibody provided herein), or a pharmaceutical composition is administered intranasally, intramuscularly, intravenously, intratumorally, or subcutaneously. The therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In some embodiments, it may be desirable to administer a therapeutic agent, or a pharmaceutical composition provided herein locally to the area in need of treatment. This may be achieved by, for example, and not by way of limitation, local infusion, by topical administration (e.g., by intranasal spray), by injection (notably, an intratumoral injection), or by means of an implant, the implant being of a porous, non-porous, or gelatinous material, including membranes, such as sialastic membranes, or fibers. In some embodiments, when administering an antibody provided herein, care must be taken to use materials to which the antibody does not absorb.

In some embodiments, a therapeutic agent provided herein can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In some embodiments, a therapeutic agent provided herein can be delivered in a controlled release or sustained release system. In some embodiments, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of a therapeutic agent (e.g., an antibody provided herein) or a composition provided herein (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., MacroMol. Sci. Rev. MacroMol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In some embodiments, the polymer used in a sustained release formulation is inert, free of leachable impurities, stable on storage, sterile, and biodegradable. In yet another embodiment, a controlled or sustained release system can be placed in proximity of the therapeutic target, e.g., the nasal passages or lungs, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising one or more antibodies provided herein. See, e.g., U.S. Pat. No. 4,526,938, PCT publication WO 91/05548, PCT publication WO 96/20698, Ning et al., 1996, “Intratumoral Radioimmunotherapy of a Human Colon Cancer Xenograft Using a Sustained-Release Gel,” Radiotherapy Et Oncology 39:179-189, Song et al., 1995, “Antibody Mediated Lung Targeting of Long-Circulating Emulsions,” PDA Journal of Pharmaceutical Science Et Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760.

In some embodiments, a composition useful in a method provided herein comprises one, two or more antibodies provided herein (e.g., an anti-VISTA antibody). In another embodiment, a composition useful in a method provided herein comprises one, two or more antibodies provided herein and a therapeutic agent other than an antibody provided herein. In some embodiments, the agents are known to be useful for or have been or are currently used for the prevention, treatment and/or alleviation of one or more symptoms of a VISTA-mediated disease, disorder or condition. In addition to therapeutic agents, the compositions provided herein may also comprise a carrier.

The compositions provided herein include bulk compositions useful in the manufacture of pharmaceutical compositions (e.g., compositions that are suitable for administration to a subject or patient) that can be used in the preparation of unit dosage forms. In some embodiments, a composition provided herein is a pharmaceutical composition. Such compositions comprise a prophylactically or therapeutically effective amount of one or more therapeutic agents (e.g., an anti-VISTA therapeutic agent, such as an anti-VISTA antibody provided herein, or other therapeutic agent), and a pharmaceutically acceptable carrier. In some embodiments, the pharmaceutical compositions are formulated to be suitable for the route of administration to a subject.

In some embodiments, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine or lignocaine to ease pain at the site of the injection. Such compositions, however, may be administered by a route other than intravenous.

The ingredients of compositions provided herein may be supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In some embodiments, an antibody provided herein is packaged in a hermetically sealed container such as an ampoule or sachette indicating the quantity of antibody. In some embodiments, the antibody is supplied as a dry sterilized lyophilized powder or water free concentrate in a hermetically sealed container and can be reconstituted, e.g., with water or saline to the appropriate concentration for administration to a subject. In some embodiments, the antibody is supplied as a dry sterile lyophilized powder in a hermetically sealed container at a unit dosage of at least 0.1 mg, at least 0.5 mg, at least 1 mg, at least 2 mg, or at least 3 mg, such as at least 5 mg, at least 10 mg, at least 15 mg, at least 25 mg, at least 30 mg, at least 35 mg, at least 45 mg, at least 50 mg, at least 60 mg, at least 75 mg, at least 80 mg, at least 85 mg, at least 90 mg, at least 95 mg, or at least 100 mg. The lyophilized antibody can be stored at between 2 and 8° C. in its original container and the antibody can be administered within 12 hours, such as within 6 hours, within 5 hours, within 3 hours, or within 1 hour after being reconstituted. In an alternative embodiment, an antibody is supplied in liquid form in a hermetically sealed container indicating the quantity and concentration of the antibody. In some embodiments, the liquid form of the antibody is supplied in a hermetically sealed container at least 0.1 mg/ml, at least 0.5 mg/ml, or at least 1 mg/ml, such as at least 5 mg/ml, at least 10 mg/ml, at least 15 mg/ml, at least 25 mg/ml, at least 30 mg/ml, at least 40 mg/ml, at least 50 mg/ml, at least 60 mg/ml, at least 70 mg/ml, at least 80 mg/ml, at least 90 mg/ml, or at least 100 mg/ml.

The amount of an anti-VISTA therapeutic agent (e.g., an anti-VISTA antibody) or a composition provided herein that will be effective in the prevention, treatment and/or alleviation of one or more symptoms of a VISTA-mediated disease, disorder or condition can be determined by standard clinical techniques.

Accordingly, a dosage of an anti-VISTA therapeutic agent (e.g., an anti-VISTA antibody) or a composition that results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention, treatment and/or alleviation of one or more symptoms of a VISTA-mediated disease, disorder or condition. In addition, in vitro assays may optionally be employed to help identify optimal dosage ranges. The precise dose to be employed in the formulation will also depend on the route of administration, and the seriousness of a VISTA-mediated disease, disorder or condition, and should be decided according to the judgment of the practitioner and each patient's circumstances.

Effective doses may be extrapolated from dose-response curves derived from in vitro or animal model test systems.

For the antibodies provided herein, the dosage administered to a patient can be, in some embodiments, 0.1 mg/kg to 100 mg/kg of the patient's body weight. In some embodiments, the dosage administered to the patient is about 1 mg/kg to about 75 mg/kg of the patient's body weight. In some embodiments, the dosage administered to a patient is between 1 mg/kg and 20 mg/kg of the patient's body weight, such as 1 mg/kg to 5 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of the antibodies provided herein may be reduced by enhancing uptake and tissue penetration of the antibodies by modifications such as, for example, lipidation.

In some embodiment, approximately 100 mg/kg or less, approximately 75 mg/kg or less, approximately 50 mg/kg or less, approximately 25 mg/kg or less, approximately 10 mg/kg or less, approximately 5 mg/kg or less, approximately 1 mg/kg or less, approximately 0.5 mg/kg or less, or approximately 0.1 mg/kg or less of an antibody provided herein is administered 5 times, 4 times, 3 times, 2 times or 1 time to prevent, treat or alleviate one or more symptoms of a VISTA-mediated disease, disorder or condition. In some embodiments, an antibody provided herein is administered about 1-12 times, wherein the doses may be administered as necessary, e.g., weekly, biweekly, monthly, bimonthly, trimonthly, etc., as determined by a physician. In some embodiments, a lower dose (e.g., 1-15 mg/kg) can be administered more frequently (e.g., 3-6 times). In other embodiments, a higher dose (e.g., 25-100 mg/kg) can be administered less frequently (e.g., 1-3 times). However, as will be apparent to those in the art, other dosing amounts and schedules are easily determinable and within the scope of the disclosure.

In some embodiments, approximately 100 mg/kg, approximately 75 mg/kg or less, approximately 50 mg/kg or less, approximately 25 mg/kg or less, approximately 10 mg/kg or less, approximately 5 mg/kg or less, approximately 1 mg/kg or less, approximately 0.5 mg/kg or less, approximately 0.1 mg/kg or less of an antibody provided herein in a sustained release formulation is administered to a subject, such as a human, to prevent, treat and/or alleviate one or more symptoms of a VISTA-mediated disease. In another some embodiment, an approximately 100 mg/kg, approximately 75 mg/kg or less, approximately 50 mg/kg or less, approximately 25 mg/kg or less, approximately 10 mg/kg or less, approximately 5 mg/kg or less, approximately 1 mg/kg or less, approximately 0.5 mg/kg or less, or approximately 0.1 mg/kg or less bolus of an antibody provided herein not in a sustained release formulation is administered to a subject, such as a human, to prevent, treat and/or alleviate one or more symptoms of a VISTA-mediated disease, disorder or condition, and after a certain period of time, approximately 100 mg/kg, approximately 75 mg/kg or less, approximately 50 mg/kg or less, approximately 25 mg/kg or less, approximately 10 mg/kg or less, approximately 5 mg/kg or less, approximately 1 mg/kg or less, approximately 0.5 mg/kg or less, or approximately 5 mg/kg or less of an antibody provided herein in a sustained release is administered to the subject (e.g., intranasally or intramuscularly) one, two, three or four times. In accordance with this embodiment, a certain period of time can be 1 to 5 days, a week, two weeks, or a month.

In some embodiments, a single dose of an antibody provided herein is administered to a patient to prevent, treat and/or alleviate one or more symptoms of a VISTA-mediated disease, disorder or condition, including one or more doses, such as two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty five, or twenty six, including at bi-weekly (e.g., about 14 day) intervals over the course of a year, wherein the dose is selected from the group consisting of about 0.1 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (e.g., each dose monthly dose may or may not be identical).

In some embodiments, a single dose of an antibody provided herein is administered to patient to prevent, treat and/or alleviate one or more symptoms of a VISTA-mediated disease, disorder or condition, including at one or more times, such as two, three, four, five, six, seven, eight, nine, ten, eleven, or twelve times, including at about monthly (e.g., about 30 day) intervals over the course of a year, wherein the dose is selected from the group consisting of about 0.1 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (e.g., each dose monthly dose may or may not be identical).

In some embodiments, a single dose of an antibody provided herein is administered to a patient to treat, prevent and/or alleviate a symptom of a VISTA-mediated disease, disorder or condition, including at one or more times, such as two, three, four, five, or six times, including at about bi-monthly (e.g., about 60 day) intervals over the course of a year, wherein the dose is selected from the group consisting of about 0.1 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (e.g., each bi-monthly dose may or may not be identical).

In some embodiments, a single dose of an antibody provided herein is administered to a patient to treat, prevent and/or alleviate one or more symptoms of a VISTA-mediated disease disorder or condition, including at one or more times, such as two, three, or four times at about tri-monthly (e.g., about 120 day) intervals over the course of a year, wherein the dose is selected from the group consisting of about 0.1 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (e.g., each tri-monthly dose may or may not be identical).

In some embodiments, the route of administration for a dose of an antibody provided herein to a patient is intranasal, intramuscular, intravenous, or a combination thereof, but other routes described herein are also acceptable. Each dose may or may not be administered by an identical route of administration. In some embodiments, an antibody provided herein may be administered via multiple routes of administration simultaneously or subsequently to other doses of the same or a different antibody provided herein.

In some embodiments, the anti-VISTA therapeutic agents (e.g., anti-VISTA antibodies) provided herein are administered prophylactically or therapeutically to a subject. Anti-VISTA therapeutic agents (e.g., an anti-VISTA antibodies) can be prophylactically or therapeutically administered to a subject so as to prevent, lessen or alleviate a VISTA-mediated disease, disorder or condition, or symptom thereof.

### Kits

Also provided herein is a pharmaceutical pack or kit comprising one or more containers filled with one or more of the ingredients of the pharmaceutical compositions provided herein, such as one or more antibodies (e.g., an anti-PSGL-1 and/or an anti-VISTA antibody) provided herein. Optionally associated with such container(s) can be a notice in the form prescribed by a governmental agency regulating the manufacture, use or sale of pharmaceuticals or biological products, which notice reflects approval by the agency of manufacture, use or sale for human administration. In some embodiments, the kit comprises a package insert. The term “package insert” is used to refer to instructions customarily included in commercial packages of therapeutic products, that contain information about the indications, usage, dosage, administration, contraindications and/or warnings concerning the use of such therapeutic products, as well as instructions for use.

Also provided herein are kits that can be used in the above methods. In some embodiment, a kit comprises an antibody (e.g., an anti-PSGL-1 and/or an anti-VISTA antibody) provided herein, such as an isolated antibody (e.g., an anti-PSGL-1 and/or an anti-VISTA antibody), in one or more containers. In some embodiments, the kits provided herein contain a substantially isolated PSGL-1 or VISTA as a control. In some embodiments, the kits provided herein further comprise a control antibody which does not react with the PSGL-1 and/or VISTA. In some embodiments, the kits provided herein contain a means for detecting the binding of a modified antibody to PSGL-1 and/or VISTA (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate). In some embodiments, the kit may include a recombinantly produced or chemically synthesized PSGL-1 and/or VISTA. The PSGL-1 and/or VISTA provided in the kit may also be attached to a solid support. In some embodiments, the detecting means of the above described kit includes a solid support to which PSGL-1 and/or VISTA is attached. Such a kit may also include a non-attached reporter-labeled anti-human antibody. In some embodiments, binding of the antibody to the PSGL-1 and/or VISTA can be detected by binding of the reporter-labeled antibody.

It is understood that modifications which do not substantially affect the activity of the various embodiments of this disclosure are also provided herein. Accordingly, the following examples are intended to illustrate but not limit the present disclosure.

### EXAMPLES

### Example I

### Identification of the Receptor to VISTA

This example describes for the first time the identification of a receptor of VISTA using CAPTIREC™, a TRICEPS™-based ligand-receptor capture system (Dualsystems Biotech AG).

The CAPTIREC™ method, with a VISTA-Fc fusion protein as the ligand of interest and anti-CD28 antibody as a control ligand, was performed on naïve T cells isolated from human primary T cells. The nucleotide and amino acid sequences of the VISTA-Fc fusion protein construct used in the below experiments are shown below:

VISTA-Fc Fusion Protein Nucleotide Sequence (underlined sequence codes for VISTA; bold sequence codes for the Fc-fragment of a human IgG1 antibody)

(SEQ ID NO: 37)
ATGGGCGTGCCCACAGCCCTGGAAGCTGGCAGCTG
GAGGTGGGGAAGCCTGCTGTTCGCCCTGTTTCTGG
CCGCCTCCCTGGGACCTGTGGCCGCCTTTAAGGTC
GCCACCCCTTACAGCCTGTACGTGTGCCCCGAGGG
CCAGAACGTGACCCTGACCTGCAGACTGCTGGGCC
CTGTGGACAAGGGCCACGACGTGACCTTCTACAAG
ACCTGGTACAGGAGCAGCAGGGGCGAGGTCCAGAC
CTGCAGCGAGAGGAGGCCCATCAGGAACCTGACCT
TCCAGGACCTGCACCTGCACCACGGAGGCCATCAG
GCCGCCAACACCTCCCACGACCTGGCTCAGAGGCA
CGGACTGGAGAGCGCCAGCGATCACCACGGCAACT
TCAGCATCACCATGAGGAACCTCACCCTGCTGGAC
AGCGGCCTGTACTGTTGCCTGGTGGTGGAGATCAG
GCACCACCACAGCGAGCACAGAGTGCACGGCGCCA
TGGAACTGCAGGTGCAGACCGGAAAGGACGCCCCC
AGCAACTGCGTGGTGTACCCCAGCAGCTCCCAGGA
CAGCGAGAACATCACCGCCGCCAGATCTGTGGAGT
GCCCACCTTGCCCAGCACCACCTGTGGCAGGACCT
TCAGTCTTCCTCTTCCCCCCAAAACCCAAGGACAC
CCTCATGATCTCCCGGACCCCTGAGGTCACATGCG
TGGTGGTGGACGTGAGCCACGAAGACCCTGAGGTC
AAGTTCAACTGGTACGTGGACGGCGTGGAGGTGCA
TAATGCCAAGACAAAGCCGCGGGAGGAGCAGTACA
ACAGCACGTACCGTGTGGTCAGCGTCCTCACCGTC
CTGCACCAGGACTGGCTGAATGGCAAGGAGTACAA
GTGCAAGGTCTCCAACAAAGGCCTCCCATCCTCCA
TCGAGAAAACCATCTCCAAAGCCAAAGGGCAGCCC
CGAGAACCACAGGTGTACACCCTGCCCCCATCCCG
GGAGGAGATGACCAAGAACCAGGTCAGCCTGACCT
GCCTGGTCAAAGGCTTCTATCCCAGCGACATCGCC
GTGGAGTGGGAGAGCAATGGGCAGCCGGAGAACAA
CTACAAGACCACGCCTCCCGTGCTGGACTCCGACG
GCTCCTTCTTCCTCTACAGCAAGCTCACCGTGGAC
AAGAGCAGGTGGCAGCAGGGGAACGTCTTCTCATG
CTCCGTGATGCATGAGGCTCTGCACAACCACTACA
CGCAGAAGAGCCTCTCCCTGTCTCCGGGTAAATGA

VISTA-Fc Fusion Protein Amino Acid Sequence (underlined sequence is VISTA; bold sequence is the Fc-fragment of a human IgG1 antibody)

(SEQ ID NO: 38)
MGVPTALEAGSWRWGSLLFALFLAASLGPVAAFKV
ATPYSLYVCPEGQNVTLTCRLLGPVDKGHDVTFYK
TWYRSSRGEVQTCSERRPIRNLTFQDLHLHHGGHQ
AANTSHDLAQRHGLESASDHHGNFSITMRNLTLLD
SGLYCCLVVEIRHHHSEHRVHGAMELQVQTGKDAP
SNCVVYPSSSQDSENIRSVECPPCPAPPVAGPSVF
LFPPKPKDTLMISRTPEVTCVVVDVSHEDPEVKFN
WYVDGVEVHNAKTKPREEQYNSTYRVVSVLTVLHQ
DWLNGKEYKCKVSNKGLPSSIEKTISKAKGQPREP
QVYTLPPSREEMTKNQVSLTCLVKGFYPSDIAVEW
ESNGQPENNYKTTPPVLDSDGSFFLYSKLTVDKSR
WQQGNVFSCSVMHEALHNHYTQKSLSLSPGK

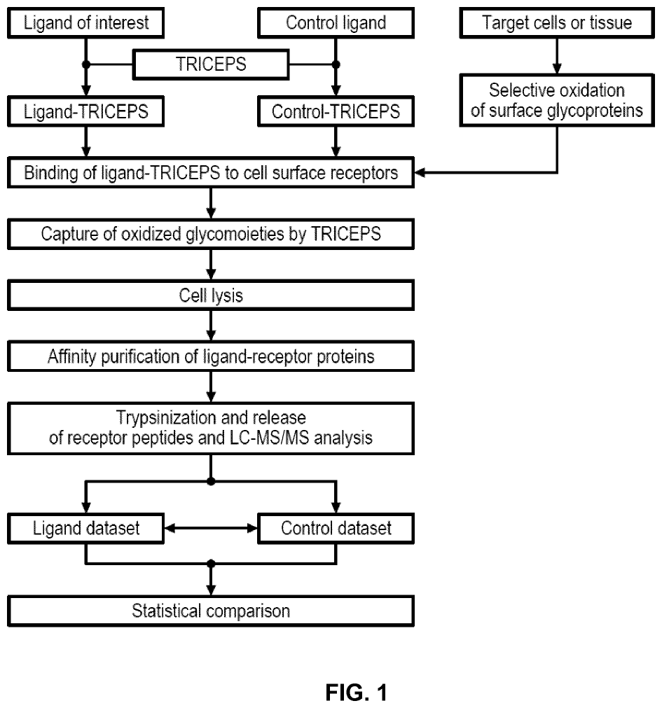

An overview of the CAPTIREC™ procedure is outlined in FIG. 1. Briefly, the VISTA-Fc fusion protein and the anti-CD28 antibody were separately coupled with TRICEPS™. Naïve human T cells were isolated from commercially available primary human T cells. The surface glycoproteins of the naïve T cells were selectively oxidized. Ligand binding and receptor coupling to the cell surface of the oxidized naïve T cells was performed. The reacted T cells were then lysed and the resulting lysate was affinity purified for the ligand-receptor protein complexes. The purified proteins where then cleaved by trypsin digestion, thereby releasing receptor peptides. The resulting receptor peptides were analyzed by liquid chromatography-tandem mass spectrometry (LC-MS/MS). To allow for statistical analysis, the experiments were done in biochemical triplicates.

The isolation of naïve T cells was performed by negatively selecting the naïve T cells from human primary T cells of healthy donors, which were purchased from ALLCELLS (Alameda, Calif.). In order to negatively select the desired cells, a Miltenyi's Naïve Pan T-cell Isolation Kit (#130-097-095) was used according to the manufacturer's protocol. Briefly, peripheral blood mononuclear cells (PBMCs) were resuspended in MACS Running Buffer and incubated for 5 min with a mixture of biotin-conjugated monoclonal anti-human antibodies against HLA-DR, CD14, CD15, CD16, CD19, CD25, CD36, CD56, CD57, CD45RO, CD123, CD244, CD235a and anti-TCR γ/δ, followed by a 10-min incubation with anti-biotin magnetic beads conjugated to monoclonal anti-CD61 and anti-biotin antibodies. Naïve T cells were then negatively selected using the autoMACS Separator (Miltenyi Biotech, San Diego Calif.). 100×10⁶ naïve T cells were used per TRICEPS' ligand capture reaction.

The remaining steps of the CAPTIREC™ procedure, which included coupling of the VISTA-Fc fusion protein or the anti-CD28 antibody to TRICEPS′, selectively oxidizing surface glycoproteins of the naïve T cells, ligand binding and receptor coupling to the cell surface of the oxidized T cells, lysing of the T cells, affinity purifying the cell lysate, and digesting with trypsin, were performed according to modified procedures described in Frei et al., Nat. Protoc., 8(7):1321-1336 (2013) and Frei et al., Nat. Biotechnol., 30(10):997-1001 (2012).

The resulting receptor peptides were analyzed by LC-MS/MS on a Thermo LTQ Orbitrap XL spectrometer fitted with an electrospray ion source. The samples were measured in data dependent acquisition mode in a 120 min gradient using a 10 cm C18 packed column. Six individual samples in the CAPTIREC™ dataset were analyzed with a statistical ANOVA model. This model assumes that the measurement error follows Gaussian distribution and views individual features as replicates of a protein's abundance and explicitly accounts for this redundancy. It tests each protein for differential abundance in all pairwise comparisons of ligand and control samples and reports the p-values. Next, p-values are adjusted for multiple comparisons to control the experiment-wide false discovery rate (FDR).

Peptide identifications were filtered to an FDR of <=1% and quantified using an MS1-based label-free approach. For the MS1 quantification, the nonlinear DYNAMICS Progenesis QI for proteomics software was used, set to consider all unique peptides. Identified proteins were filtered for the association with the terms membrane, secreted, glycosylation, using the information provided by Uniprot. Protein identifications relaying upon only one peptide were not considered for the analysis.

The processed CAPTIREC™ data was graphed in the form of a volcano plot on the protein level, which plots fold change versus statistical significance. The adjusted p-value obtained for every protein is plotted against the magnitude of the fold enrichment between the two experimental conditions. The receptor candidate space is defined by an enrichment factor of >4 fold and statistical significance (FDR-adjusted p-value<0.01).

Of the observed glycoproteins, CD28 was identified with 5 peptides in the control dataset. This indicated a successful CAPTIREC™ workflow. PSGL-1 was identified with 6 peptides and VISTA itself was also identified with 12 peptides in the VISTA-Fc fusion protein dataset (see Table 6).

TABLE 6

| | Protein | Log2 | AdJ. p- |
|---|---|---|---|
| Gene Name | Name | FC | value |
| SELPLG | PSGL-1 | 2.33 | 1.83E−13 |
| Chromosome 10 Open | VISTA | 7.38 | 0 |
| Reading Frame 54 | | | |

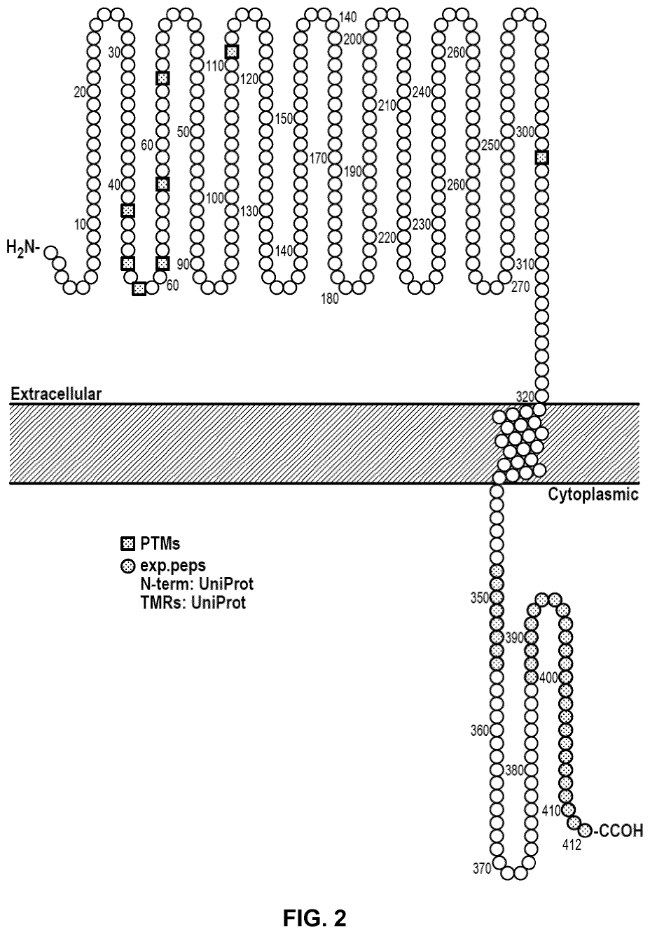

For the identified binding partner, PSGL-1, a Protter illustration was generated (FIG. 2), which annotates N-glycosylation sites (residues surrounded by squares) and the experimentally observed peptides (filled in circles) (Omasits et al., Bioinformatics: advance online pub., October, 2013). This mapping shows that all six of the peptides detected localize to the intracellular domain of PSGL-1. Analysis of the extracellular domain of PSGL-1 reveals that there are few tryptic peptide cleavage sites in the extracellular domain despite the size of the domain. PSGL-1 contains three N-glycosylation sites and peptides with these sites would have been lost from the LC-MS/MS analysis described above. The remaining potential peptides are either too large, too small or become processed during protein sorting, providing a rationale for the finding that only peptides mapping to the intracellular domain of PSGL-1 were detected.

In view of the above analysis, PSGL-1 was determined to be the heterophilic binding partner to VISTA. Because VISTA has been previously shown to be a broad-spectrum negative checkpoint regulator that is expressed on hematopoietic cells (Lines et al., Cancer Res., 74(7)1924-1932 (2014)), the above results show that the VISTA interaction with PSGL-1 likely leads to suppression of T cells. Therefore, interfering with (e.g., inhibiting or blocking) that interaction with agents that target PSGL-1 and/or VISTA, such as anti-PSGL-1 and/or anti-VISTA antibodies, can lead to reinvigoration or de novo activation of the immune system through activated T cells. This activation of the immune system will result in a highly beneficial physiological response in the treatment of a VISTA mediated disease, disorder or condition, including in the context of cancer treatment (e.g., hematological cancer treatment).

### Example II

### Binding of VISTA to PSGL-1

This example describes the binding properties of VISTA to PSGL-1.

A VISTA-Fc fusion protein (e.g., described in Example I) was immobilized to a solid surface and assayed for its binding to the extracellular domain of PSGL-1. For these experiments, two different constructs were generated containing the extracellular domain of PSGL-1. Both constructs were fused to IgG kappa signal sequence and an Fc fragment. Additionally, a propeptide sequence or tandem repeat unit, which can be important for proper functioning, was added (see, e.g., Cummings R. D., Brazilian J Med Biol Res, 32:519-28 (1999)). The cells were co-transfected with the constructs expressing GCNT1 and FUT3 glycosyltransferases to ensure proper post-translational modification of the protein, which was known to be important for high-affinity binding of PSGL-1 to P-selectin (Sako et al., Cell, 75(6):1179-86 (1993); Yang 5 et al., Thrombosis and Haemostasis, 81(1):1-7 (1999); Carlow et al., Immunol Rev 230(1):75-96 (2009); Kumar et al., Blood, 88(10):3872-9 (1996); Cummings R. D., Brazilian J Med Biol Res, 32:519-28 (1999)). The amino acid sequences of the PSGL-1 constructs are shown below:

PSGL-1 construct A—Amino acid sequence (Fc-fused PSGL-1 with IgG kappa signal sequence and propeptide sequence). IgG kappa signal sequence=italic; Propeptide sequence=bold; Fc sequence=underline.

(SEQ ID NO: 39)
MPLQLLLLLILLGPGNSLQLWDTWADEAEKALGPL
LARDRRQATEYEYLDYDFLPETEPPEMLRNSTDTT
PLTGPGTPESTTVEPAARRSTGLDAGGAVTELTTE
LANMGNLSTDSAAMEIQTTQPAATEAQTTPLAATE
AQTTRLTATEAQTTPLAATEAQTTPPAATEAQTTQ
PTGLEAQTTAPAAMEAQTTAPAAMEAQTTPPAAME
AQTTQTTAMEAQTTAPEATEAQTTQPTATEAQTTP
LAAMEALSTEPSATEALSMEPTTKRGLFIPFSVSS
VTHKGIPMAASNLSVARSVECPPCPAPPVAGPSVF
LFPPKPKDTLMISRTPEVTCVVVDVSHEDPEVKFN
WYVDGVEVHNAKTKPREEQYNSTYRVVSVLTVLHQ
DWLNGKEYKCKVSNKGLPSSIEKTISKAKGQPREP
QVYTLPPSREEMTKNQVSLTCLVKGFYPSDIAVEW
ESNGQPENNYKTTPPVLDSDGSFFLYSKLTVDKSR
WQQGNVFSCSVMHEALHNHYTQKSLSLSPGK

PSGL-1 construct B—Amino acid sequence (Fc-fused PSGL-1 with IgG kappa signal sequence and tandem repeat unit). IgG kappa signal sequence=italic; Tandem repeat unit=bold; Fc sequence=underline.

(SEQ ID NO: 40)
METDTLLLWVLLLWVPGSTGDQATEYEYLDYDFLP
ETEPPEMLRNSTDTTPLTGPGTPESTTVEPAARRS
TGLDAGGAVTELTTELANMGNLSTDSAAMEIQTTQ
PAATEAQTTQPVPTEAQTTPLAATEAQTTRLTATE
AQTTPLAATEAQTTPPAATEAQTTQPTGLEAQTTA
PAAMEAQTTAPAAMEAQTTPPAAMEAQTTQTTAME
AQTTAPEATEAQTTQPTATEAQTTPLAAMEALSTE
PSATEALSMEPTTKRGLFIPFSVSSVTHKGIPMAA
SNLSVNYPVGAPDHISV ARSVECPPCPAPPVAGP
SVFLFPPKPKDTLMISRTPEVTCVVVDVSHEDPEV
KFNWYVDGVEVHNAKTKPREEQYNSTYRVVSVLTV
LHQDWLNGKEYKCKVSNKGLPSSIEKTISKAKGQP
REPQVYTLPPSREEMTKNQVSLTCLVKGFYPSDIA
VEWESNGQPENNYKTTPPVLDSDGSFFLYSKLTVD
KSRWQQGNVFSCSVMHEALHNHYTQKSLSLSPGK

For these experiments, PSGL-1 construct A (PSGL-1A) and construct B (PSGL-1B) were tested against immobilized VISTA-Fc.

The immobilized VISTA-Fc sample was equilibrated in HEPES buffered saline (HBS) containing calcium (1.5 mM calcium chloride) and magnesium (1.0 mM magnesium chloride). The same buffer was used as running buffer. Samples containing the PSGL-1A or PSGL-1B, which also contained calcium and magnesium, were flowed across a solid surface containing the immobilized VISTA-Fc at a flow rate of 60 μl/min, with 120 seconds of contact time and 300 seconds for dissociation after the surface was regenerated with a 3 second pulse of glycine (pH 1.5). Six difference concentrations of PSGL-1A and PSGL-1B were assayed (0.3 μM, 0.60 μM, 1.20 μM, 2.4 μM, 4.8 μM and 9.6 μM).

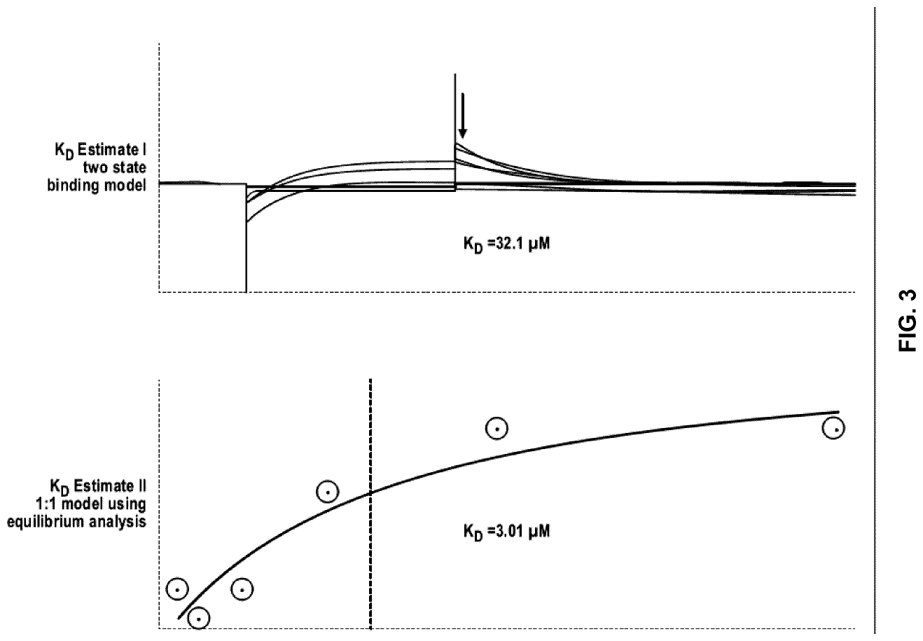

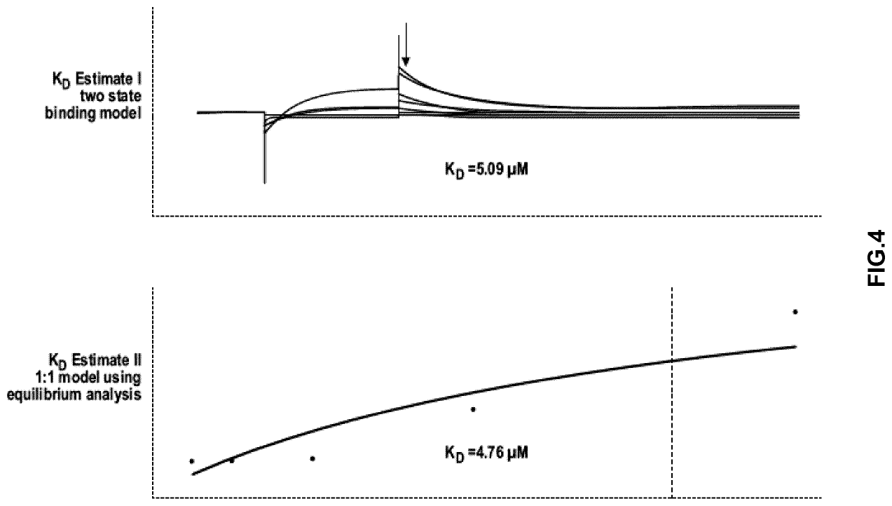

Two different analyses of the experiments were performed: (1) a two-state binding model; and (2) an equilibrium affinity analysis (1:1 model). For PSGL-1A, the two-state binding model showed a binding affinity (K_{D}) of 32.1 μM, whereas the 1:1 model showed a K_{D} of 3.01 μM (FIG. 3). For PSGL-1B, the two-state binding model showed K_{D} of 5.09 μM, whereas the 1:1 model showed a K_{D} of 4.76 μM (FIG. 4).

The above analyses showed that there was a net signal response for both PSGL-1A and PSGL-1B constructions. The resulting sensograms show features associated with binding and dissociation. It is noted that the affinity estimations were qualitative. A quantitative estimate of binding may be possible when the surface activity is great than the activity of the present experiments (e.g., >0.5% with a purified PSGL-1 construct). Additionally, in these experiments using injections of PSGL-1A resulted in sample carryover in subsequent runs. In these experiments, the estimated affinities between VISTA and PSGL-1 were comparably similar, PSGL-1A (about 3 μM) and PSGL-1B (about 5 μM).

### Example III

### Binding of VISTA to PSGL-1 on Cells

This example describes the binding of VISTA to PSGL-1 expressed by a promyelocytoic cell line (HL-60; ATCC, CCL-240) using a bifunctional crosslinking approach.

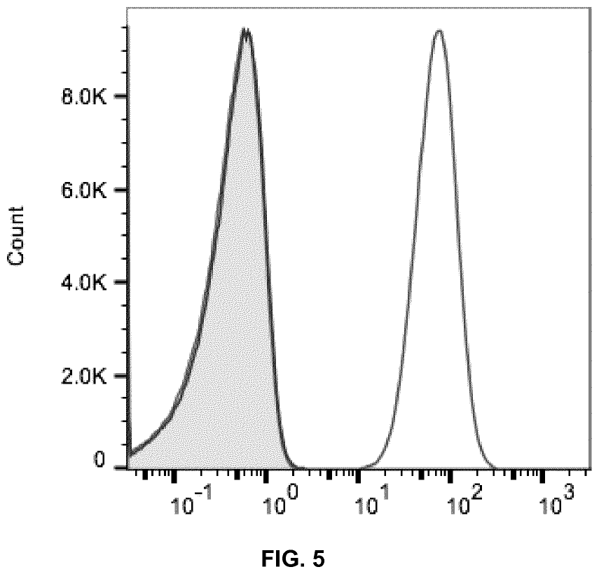

In these experiments, expression of PSGL-1 was assessed using a PE-conjugated anti-PSGL-1 monoclonal antibody (Abcam; ab78188) designated KPL-1. Expression of PSGL-1 by HL-60 cells was detected by flow cytometry using standard methods (FIG. 5). The copy number of the PSGL-1 protein was estimated to be approximately 263,000±2,800 per cell.

Also in these experiments, samples of VISTA-Fc fusion protein (described in Example I) and negative controls of an anti-CD28 antibody (BioXcell, BE0248) and IgG1-Fc (RED Systems; 110-HG-100) were covalently coupled to a bifunctional linker (Sulfo-SBED—ThermoFisher Scientific; 33073) using the manufacturing recommended conditions. The resulting samples were incubated with HL-60 cells for 30 min. at room temperature in a dark place. Crosslinking was photoactivated with a UV light source for 20 min. Cells were then lysed and a Protein A Sepharose pull-down was performed. A Western Blot using an anti-PSGL-1 polyclonal antibody (RED Systems; AF3345) or Streptavidin-HRP was performed on the samples.

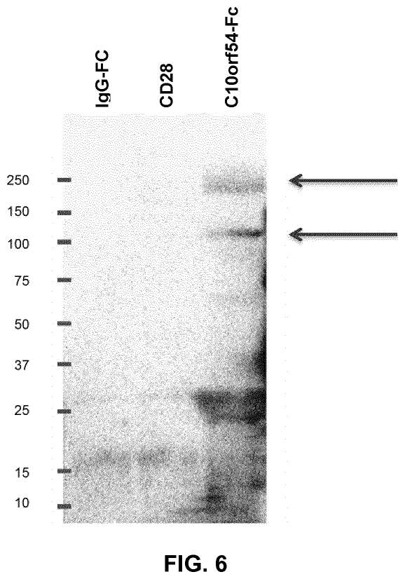

As show in FIG. 6, the VISTA-Fc interacts with PSGL-1, but not the negative isotype control IgG-Fc or the anti-CD28 antibody.

Additional experiments were performed confirming the specificity of this interaction. The above experiment was repeated, except prior to incubation of the Sulfo-SBED labeled proteins with the HL-60 cells, an anti-VISTA monoclonal antibody was added to the cells. Analysis was conducted using ImageQuant.

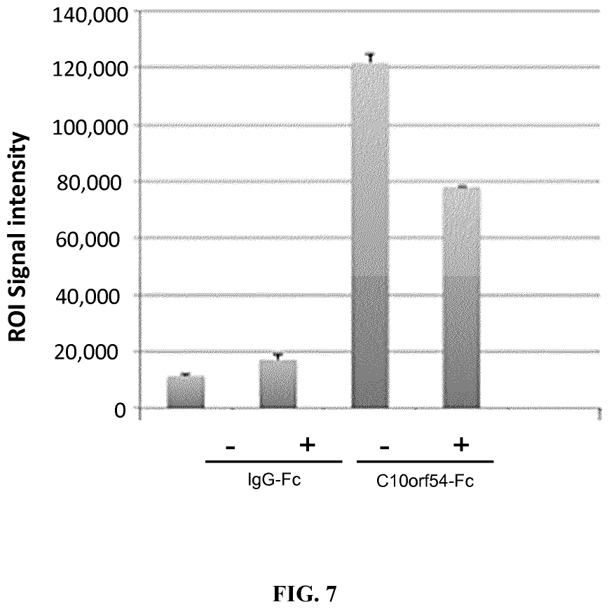

As shown in FIG. 7, inoculation with an anti-VISTA antibody resulted in attenuation of the interaction between VISTA and PSGL-1.

These experiments show that PSGL-1 expressed on HL-60 cells is a binding partner for VISTA. These experiments also show that this interaction is specific and attenuated by anti-VISTA blocking antibodies.

### Example IV

### Binding of VISTA to PSGL-1 on PBMCs

This example describes the binding of VISTA to PSGL-1 expressed by peripheral blood mononuclear cells (PBMCs) using a crosslinking approach.

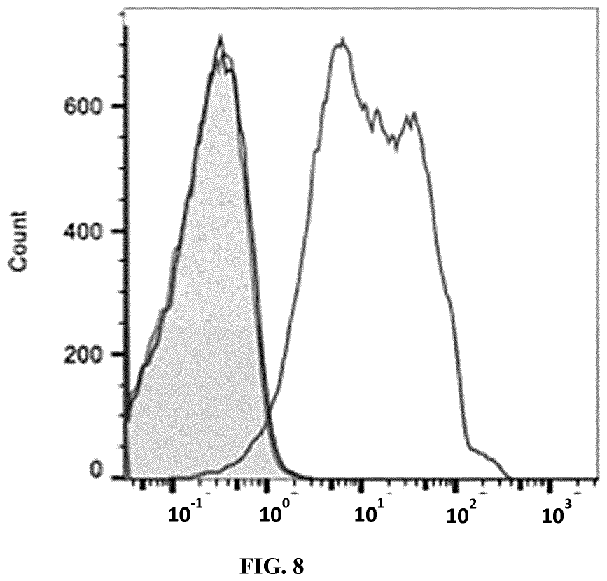

In these experiments, expression of PSGL-1 was assessed using a PE-conjugated anti-PSGL-1 monoclonal antibody (Abcam; ab78188) designated KPL-1. Expression of PSGL-1 by PBMCs was detected by flow cytometry using standard methods (FIG. 8). The copy number of the PSGL-1 protein was estimated to be approximately 38,000 per cell.

Additionally, PBMCs were either left untreated or incubated with a crosslinker (10 mM BS³; ThermoFisher Scientific; 21580) for 90 min. on ice. Following quenching of the crosslinking reaction where needed, cells were lysed and the resulting lysates were precleared with Herceptin and GammaBind Plus Sepharose (GE Healthcare; 17-0886-01). The resulting samples were either immunoprecipitated with anti-VISTA antibody or an anti-PSGL-1 antibody (KPL-1) overnight. The immunoprecipitated samples were assayed by Western Blot using an anti-PSGL-1 polyclonal antibody (RED Systems; AF3345).

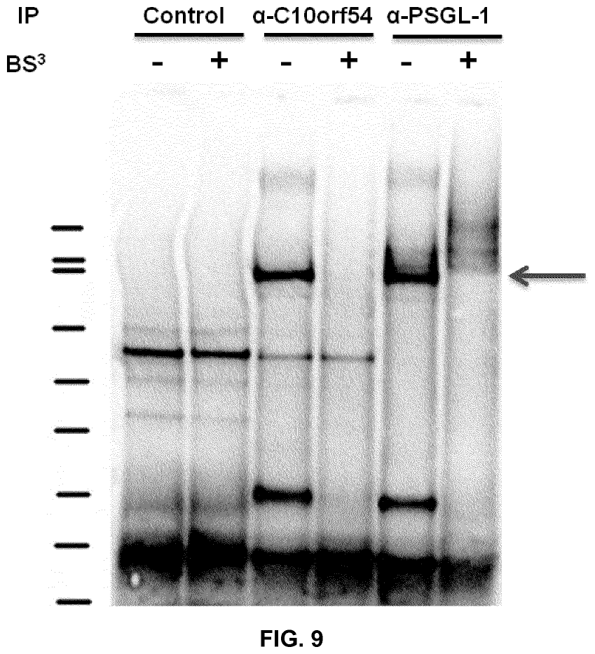

As shown in FIG. 9, line 4, after crosslinking, no PSGL-1-specific bands were detected after immunoprecipitation with an anti-VISTA antibody. For example, this might be due to blocking specific epitopes upon VISTA-PSGL-1 complex formation, and hence preventing immunoprecipitation. The anti-PSGL-1 antibody precipitated several higher molecular weight complexes (˜250-450 kDa) in BS³-treated PBMCs (FIG. 9, last lane), which were also PSGL-1-positive.

In these experiments, both anti-VISTA and anti-PSGL-1 antibodies precipitated a protein of −240 kDa from PBMCs not treated with either of the crosslinkers. This complex was PSGL-1-positive, demonstrating that PSGL-1 interacts with VISTA (FIG. 9, lanes 3 and 5). Immunoprecipitation with isotype control antibody did not produce such a band (FIG. 9, lanes 1 and 2).

These experiments show that PSGL-1 expressed on PBMCs is a binding partner for VISTA.

### Example V

### Expression of PSGL-1

This example describes the expression of PSGL-1 in various T cell subsets.

In these experiments, expression of PSGL-1 was assessed using a PE-conjugated anti-human CD162 antibody. The following T cell subsets were assessed: naïve Et resting cells (e.g., reported phenotype: CD45RO⁻/CD45RA⁺/CCR7⁺/CD62L⁺/CD27⁺/CD28⁺/CD127⁺), effector cells (e.g., reported phenotype: CD45RO⁺/CD57⁺/CD279⁻/CD95⁺/CCR7⁻/CD62L⁻), exhausted effector cells (e.g., reported phenotype: CD45RO⁺/CD57⁺/CD279⁺/CD95⁺/CD45RA⁻/CCR7⁻/CD62L⁻) and circulating memory cells (e.g., reported phenotype: Central: CD45RO⁺/CD45RA⁻/CCR7⁺/CD62L⁺, or Effector: CD45RO⁺/CD45RA⁻/CCR7⁻/CD62L⁺).

In these experiments, human PBMC samples were obtained from ALLCELLS (Emeryville, Calif.). Two T cell marker panels were prepared as follows:

a. Panel 1: T cell markers+Effector/Exhausted effector specific markers
i. CD45RA-FITC
ii. CD45RO-PerCP-eFluor 710
iii. CD197 (CCR7)-Brilliant Violet 510
iv. CD62L-APC-eFluor 780
v. CD57-Pacific Blue
vi. CD95 (Fas)-PE-Cy7
vii. CD279-APC
viii. CD162-PE

b. Panel 2: T cell markers+Naïve/resting specific markers
ix. CD45RA-FITC
x. CD45RO-PerCP-eFluor 710
xi. CD197 (CCR7)-Brilliant Violet 510
xii. CD62L-APC-eFluor 780
xiii. CD27-Pacific Blue
xiv. CD28-PE-Cy7
xv. CD127-APC
xvi. CD162-PE

In these experiments, approximately 1E6 cells and FcR Blocking Reagent (Miltenyl-Biotec, 130-092-247) were added to each panel along with the appropriate isotype controls for each antibody and incubated for 30 min. at 4° C. in the dark. Cells were then washed and the Median Fluorescence Intensities (MFI) were calculated for each sample on a MACSQuant Analyzer. MFI values were quantified using the Quantum Simply Cellular anti-Mouse IgG Kit (Bangs Laboratories, 815B). 1E5 events per fluorophore were collected from the viable population (DAPI negative) and exported for analysis in FlowJo.

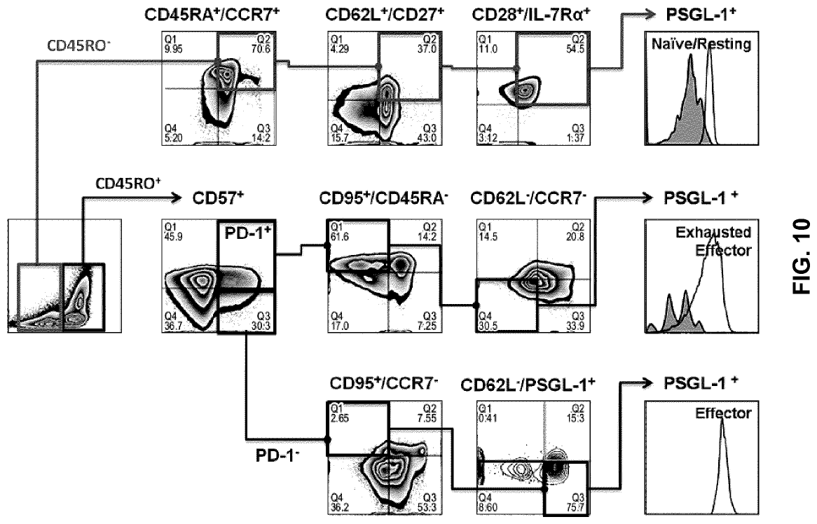

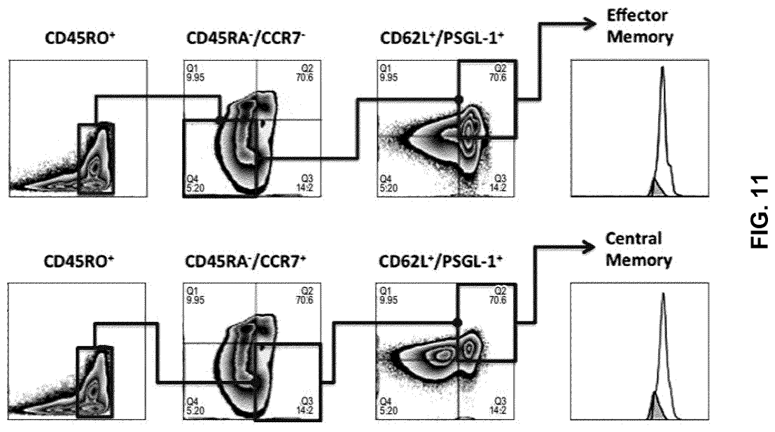

As shown in FIGS. 10 and 11, PSGL-1 was present in the naïve/resting, effector, exhausted effector, as well as both circulating central and effector T cell subsets. Also shown in FIGS. 10 and 11, PSGL-1 expression was elevated in effector subtypes relative to naïve and exhausted T cells. The highest level of expression was in the effector subset, whereas the lowest level of expression was in the naïve/resting subset. Table 7 shows copy numbers for the PSGL-1 expression in each subset.

TABLE 7

| | | Copy Number |
|---|---|---|
| T cell Subset | | [Mean ± SD] |
| Naïve/Resting | | 30005 ± 24564 |
| Effector | | 221660 ± 44470 |
| Exhausted effector | | 43544 ± 18769 |
| Circulating | Central | 82528 ± 32176 |
| memory | Effector | 82041 ± 16489 |

These experiments show that PSGL-1 is differentially expressed across various T cell subsets in human PBMCs.

### Example VI

### In Silico Analysis of VISTA and PSGL-1 Expression

This example shows that VISTA expression is correlated with PSGL1 in several indications.

The Cancer Genome Atlas (TCGA) provides comprehensive analysis of cancer genome profiles with high-throughput technologies, including next-generation sequencing and microarray-based methods. The TCGA deposited data contains information about both nucleotide sequence and gene expression. The cBioportal site for Cancer Genomics (http://www.cbioportal.org/) thus provides visualization, analysis and download of large-scale cancer genomics data sets. It includes genomics, transcriptomics studies from TCGA.

For each TCGA indication, the cBioportal site was queried to identify the mRNAs whose expression correlates most with VISTA's. This correlation analysis was performed using Spearman test. Statistical results (p value<0.05) are reported in Table 8; mRNAs are ranked based on their correlation with VISTA.

TABLE 8

| | PSGL1 (SEPLG) | | VSIG3 (IGSF11) | | VSIG8 | |
|---|---|---|---|---|---|---|
| | Spearman | | Spearman | | Spearman | |
| INDICATION | corr coeff | Rank* | corr coeff | Rank* | corr coeff | Rank* |
| adNSCLC | 0.72 | 1 | −0.02 | 8812 | 0.07 | 14042 |
| (LUAD) | | | | | | |
| Endometrial | 0.72 | 2 | −0.02 | 9150 | 0.01 | 8031 |
| (UCEC) | | | | | | |
| Melanoma | 0.8 | 7 | −0.31 | 19751 | 0.05 | 7401 |
| (SKCM) | | | | | | |
| sqNSCLC | 0.68 | 9 | −0.24 | 17909 | 0.03 | 7886 |
| (LUSC) | | | | | | |
| Liver | 0.62 | 10 | 0.13 | 4558 | 0.15 | 5208 |
| (LIHC) | | | | | | |
| Kidney | 0.49 | 34 | −0.11 | 16030 | −0.17 | 9336 |
| (KIRC) | | | | | | |
| Colorectal | 0.66 | 35 | 0.17 | 3931 | −0.04 | 13252 |
| (COAD) | | | | | | |
| Bladder | 0.41 | 35 | −0.22 | 18573 | −0.12 | 14214 |
| (BLCA) | | | | | | |

Table 8 shows that PSGL1 expression correlates highly with VISTA expression in several cancers. The correlation is highest in NSCCLC. By comparison, other putative receptors (i.e., VSIG3 and VSIG8) showed only poor correlation.

### Example VII

Evaluation of VISTA and PSGL1 mRNA Expression with RNA Scope

This example shows the pattern of mRNA expression and colocalization of VISTA and PSGL1 in lung squamous cell carcinoma (SCC) and adenocarcinoma (ADK) tissue microarrays (TMA).

### Materials and Methods

Paraffin-embedded lung SCC and ADK TMA blocks (3 blocks each) were freshly sectioned and processed to glass slides before the in situ hybridization (ISH) technical steps were carried out. Dissected tissue samples were placed in fresh 10% neutral buffered formalin (NBF) for 16-32 hours at room temperature (RT). The sample were then dehydrated, embedded in paraffin, and cut into 5±1 μm sections which were then mounted on Superfrost® Plus slides. The slides were baked in a dry oven for 1 hour at 60° C.

Tissue sections in 5-μm thickness were deparaffinized in xylene, followed by dehydration in an ethanol series. Tissue sections were then incubated in citrate buffer (10 nmol/L, pH 6) maintained at a boiling temperature (100° C. to 103° C.) using a hot plate for 15 minutes, rinsed in deionized water, and immediately treated with 10 μg/mL protease (Sigma-Aldrich, St. Louis, Mo.) at 40° C. for 30 minutes in a HybEZ hybridization oven (Advanced Cell Diagnostics, Hayward, Calif.).

The treated tissue sections were then hybridized with the PSGL-1 or VISTA probes using the RNAscope® 2.5 Assay (Advanced Cell Diagnostics, Hayward, Calif.), following the manufacturer's instructions.

The slides were stained with haematoxylin and eosin to perform a quality check and a microscopic evaluation of each core. The examination was performed using a standard light microscope at 20-40× magnification. Excel sheet was used for data acquisition.

Negative and positive control checks were generated using 2 specific probes. These scores were assessed to confirm the absence of contamination (negative probes) and presence of ubiquitous mRNA (positive probes). The protocol was run manually.

The labeled tissues were evaluated using a semi-quantitative method with a double scoring system for each target.

The tissue distribution scoring system ranged from 0 to 3 and represented the extent of positive cells within the population (immune infiltrate) and was considered as an immunoscore.

Immunoscore grading system: ranged from 0 to 3 as follows:
0: absent
1: low
2: moderate
3: high

The ACD grading system, i.e., the grading system recommended by the RNAscope® 2.5 Assay manufacturer (Advanced Cell Diagnostics, Hayward, Calif.), was used to estimate the quantity of RNA dots within cells. Each dot represents a single RNA molecule, as RNAscope detects individual RNA molecules. This system ranged from 0 to 4 related to the number of dots and/or clusters (0: no dots; 4: numerous dots and clusters) in the cytoplasm and could be considered as an intracellular scoring system.

### Results

### Lung SCC TMA

Three lung SCC TMAs were analyzed. A triplicate of cores was present for each patient.

TABLE 9

| Analyzed samples | Before QC | Following QC |
|---|---|---|
| Number of patients | 55 | 37 |
| Number of cores | 165 | 68 |

Labeling of both VISTA and PSGL-1 mRNAs was mostly observed in the tumor microenvironment (immune cell infiltrates). Positive-labeled cells showed a myeloid morphology (related to macrophages). However, some positive dots were occasionally noted in lymphocytes (both mRNAs) and neutrophils (VISTA only).

VISTA mRNA was predominant within tumor microenvironment infiltrates. All lung SCC cores expressed this target in some extent. Dots appeared small and numerous within the cytoplasm. Occasionally, endothelial cells displayed VISTA dots. Positive mRNA VISTA tumor cells were observed in more than 80% of cores.

Compared to VISTA, PSGL-1 mRNA expression was lower within tumor microenvironment infiltrates. Dots appeared larger but were fewer within cytoplasm than VISTA dots. Tumor cells occasionally expressed PSGL-1 mRNA (30% of cores); most of the time, the ACD grade (i.e., number of dots within cytoplasm) was quite low.

Almost all the cores displayed a moderate-to-high positive VISTA-mRNA-labeling compared to 35% for PSGL-1 mRNA. None of the cores appeared negative for both targets, i.e., each of the cores was positive for either VISTA, PSGL-1, or both. The results are summarized in Table 10.

TABLE 10

| Lung SCC | High | Moderate | Low | Absent |
|---|---|---|---|---|
| VISTA mRNA | 65% | 29% | 6% | 0% |
| immunoscore (% of cores) | | | | |
| PSGL-1 mRNA | 13% | 52% | 35% | 0% |
| immunoscore (% of cores) | | | | |

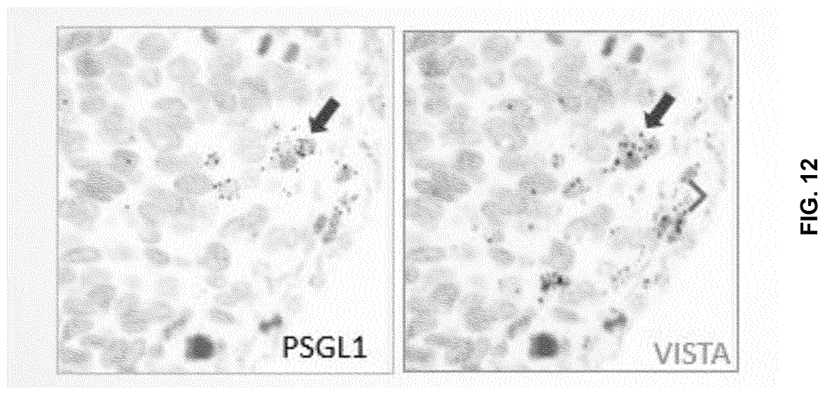

In conclusion:
VISTA and PSGL-1 mRNA colocalization was observed within tumor microenvironment, an example of which is shown in FIG. 12;
almost all the PSGL1-positive cells were adjacent to VISTA-positive cells;
in each core, at least some cells expressing both PSGL-1 and VISTA were observed;
VISTA-positive cells could be observed with no adjacent PSGL-1-positive cells. Some VISTA-positive cells could be observed with no apparent adjacent PSGL-1 positive cells. However, no PSGL-1-positive cell with no adjacent VISTA-positive cell was observed, meaning that PSGL-1-positive cells were always adjacent to VISTA-positive cells. This was partly due to the fact that VISTA-positive cells were predominant in the immune infiltrate.

### Lung ADK TMA

Three lung ADK TMA were analyzed. A quadruplicate of cores was present for each patient.

TABLE 11

| Analysed samples | Before QC | Following QC |
|---|---|---|
| Number of patients | 31 | 25 |
| Number of cores | 124 | 61 |

As for lung SCC, VISTA and PSGL-1 mRNA labeling were noted in the tumor microenvironment (immune cell infiltrate). Positive labeled cells showed a myeloid morphology (related to macrophages). However, some positive dots were occasionally noted in lymphocytes (both targets) and neutrophils (VISTA only).

The pattern of expression for both VISTA and PSGL-1 was very similar in lung ADK to what was observed in lung SCC.

More than 50% of the cores displayed a high-positive labeling for VISTA mRNA while approximately 50% of cores showed positive PSGL1 mRNA labeling. For PSGL-1, a few cores appeared negative (7%). The results are summarized in Table 12.

TABLE 12

| Lung SCC | High | Moderate | Low | Absent |
|---|---|---|---|---|
| VISTA mRNA immunoscore | 58% | 21% | 21% | 0% |
| (% of cores) | | | | |
| PSGL-1 mRNA | 18% | 47% | 28% | 7% |
| immunoscore (% of cores) | | | | |

Similar colocalization or relationship patterns between VISTA and PSGL-1 mRNA were observed in lung ADK as in lung SCC.

Semi-quantitative analysis of VISTA and PSGL-1 mRNA expression patterns by dual RNAscope revealed that the targets were frequently colocalized or expressed within adjacent cells in tumor microenvironment. VISTA mRNA appeared to be more expressed than PSGL1. However, all lung SCC cores and 83% of lung ADK cores expressed both targets.

RNAscope reveals that PSGL-1 can be expressed in the same cells or in vicinity of VISTA expressing cells

### Example VIII

IL-2 Release from CD4⁺ T Cells in Presence of PSGL-1Fc+/− Anti VISTA or Anti-PSGL1 Antibodies (72 h)

This example describes the PSGL-1-mediated inhibition of T cell activation.

### Methods:

The experiments were done in triplicates.

CD4⁺ T cells were isolated from two human healthy donors by negative selection using Milenyi Kits.

An anti-CD3 antibody (commercialized by eBiosciences, BioxCell ref 13E0001-2 clone OKT3 lot 640417J1 (mIgG2a)) was coated in 96 wells plates at concentration of 2.5 μg/ml in 100 μl for 4 h at 37° C. Then the plates were washed 2 times with PBS. The C9G4 antibody and the PSGL-1-Fc fusion protein were coated overnight at 4° C. at a concentration 224 nM in 100 μl in triplicates.

The plates were washed 4 times with PBS. 100.000 CD4⁺ T cells we added to each well in 200 μl medium containing an anti-CD28 antibody (2.5 μg/ml) and with or without the anti-VISTA antibody 26A (10 μg/ml) which is described in WO 2014/197849 (more specifically, the antibody used was a humanized antibody having the following CDR sequences, as described in WO 2014/197849: CDRH1: SEQ ID No 1297, CDRH2: SEQ ID No 1559, CDRH3: SEQ ID No 1394, CDRL1: SEQ ID No 1432, CDRL2: SEQ ID No 1477, and CDRL3: SEQ ID No 1499, or the control antibody C9G4 (described in WO 2015/162292A).

After a 72 h-incubation, the supernatants were removed and spun for 5 minutes at 1200 rpm. After centrifugation, the supernatants were transferred to a new 96 well plates and frozen at −80° C. until IL-2 was assayed.

IL-2 concentration in the supernatants was measured using a commercial kit (BD™CBA Human IL2 Flex Set, Ref #558270).

### Results:

In order to test the immunosuppressive properties of PSGL-1, the activation status of T cells was examined following stimulation in the presence or absence of the protein. To this end, a PSGL-1-Fc fusion protein was first engineered, consisting of the extracellular domain of PSGL-1 and the Fc region of human IgG. CD4⁺ T cells were then activated by anti-CD3 antibodies and CD28 in the presence PSGL-1-Fc or a control IgG. IL-2 release was monitored, as a marker of the activation of these cells.

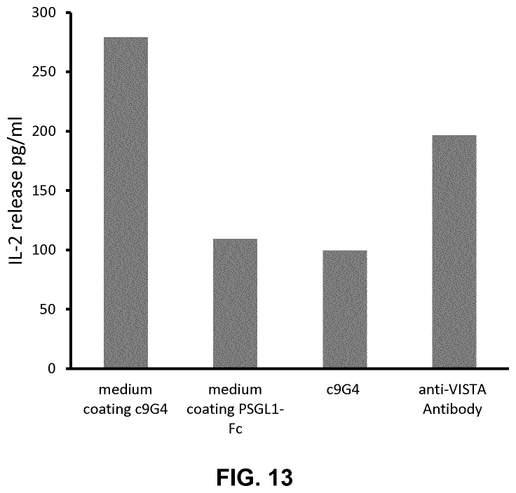

As shown in FIG. 13, incubation of T cells in the presence of PSGL-1 triggers a 2-fold reduction of IL-2 release, a marker of T cell activation, when compared with the control, i.e. an irrelevant protein coated at the same concentration (c9G4). The results obtained were similar for the two different donors. Thus PSGL-1 inhibits T-cell activation.

Addition of anti-VISTA antibodies partially reverts this inhibition (see FIG. 13). Indeed, more than 50% of the inhibition was relieved by the anti-VISTA antibodies. The addition of the control antibody (c9G4) did not affect the inhibition of IL-2 release by PSGL1-Fc, emphasizing the specificity of the effect observed with the anti-VISTA antibodies. This specific reversion upon addition of anti-VISTA antibodies demonstrates that the PSGL-1-dependent inhibition of T-cell activation is at least partially mediated by VISTA.

These results confirm that VISTA and PSGL1 interact both physically and functionally. The disruption of this interaction (here with an anti-VISTA antibodies) enhance the IL-2 release and thus the T cells activation.

1. An in vitro method for diagnosing and treating a VISTA-mediated tumor in a subject, the method comprising the steps of:
a) obtaining a biological sample from a subject,
b) contacting the biological sample of the subject with a reagent-capable of binding specifically to PSGL-1 nucleic acid or protein,
c) quantifying the binding of the reagent to PSGL-1 nucleic acid or protein in the biological sample,
d) diagnosing a VISTA-mediated tumor in the subject when binding of the reagent to PSGL-1 nucleic acid or protein is quantified, and
e) administering an effective amount of at least one anti-VISTA therapeutic agent to the diagnosed subject.

2. The method of claim 1, wherein the reagent is selected from the group consisting of a DNA probe, an RNA probe, and an anti-PSGL-1 antibody.

3. The method of claim 1, wherein the binding of PSGL-1 in immune infiltrates of a tumor microenvironment is quantified.

4. The method of claim 1, further comprising a step of scoring the tumor by comparing the level of binding quantified in step c) to an appropriate scale based on two parameters which are the intensity of staining and the percentage of positive cells.

5. The method of claim 1, further comprising a step of comparing the level of expression of step c) with a reference level, wherein an increase in the assayed level of PSGL-1 in step c) compared to the reference level is indicative of a VISTA-mediated tumor.

6. The method of claim 5, wherein the reference level is the level of expression of PSGL-1 in normal tissue samples.

7. The method of claim 5, wherein:
step b) further comprises measuring the level of expression of at least one of VISTA, CD11b, CD33, CD4, and CD8 by the immune infiltrates in the biological sample; and
step c) comprises comparing the level of expression of step b) with a control level,
whereby an increase in the assayed level of PSGL-1 and/or VISTA, CD11b, CD33, CD4, or CD8 compared to the control level of the PSGL-1 and/or VISTA, CD11b, CD33, CD4, or CD8, is indicative of a VISTA-mediated cancer.

8. The method of claim 5, wherein the VISTA-mediated tumor is selected from the group consisting of hematological cancers, bladder, breast, colon, connective tissue, rectal, gastric, esophageal, lung, larynx, kidney, oral, ovarian, or prostate cancers, or sarcomas, melanomas, or gliomas, or metastases of any of these cancers.

9. (canceled)

10. The method of claim 1, wherein the anti-VISTA therapeutic agent is an anti-VISTA antibody.

11. The method of claim 10, wherein the anti-VISTA antibody is selected in the group consisting of:
a) an anti-VISTA antibody, the antibody comprising a heavy chain comprising 3 CDRs of sequences SEQ ID NOs: 1296, 1354, and 1393, as defined by Kabat; and a light chain comprising 3 CDRs of sequences SEQ ID NOs: 1432, 1477, and 1499, as defined by Kabat; and
b) an anti-VISTA antibody, the antibody comprising a heavy chain comprising 3 CDRs of sequences SEQ ID NOs: 1296, 1559, and 1393, as defined by Kabat; and a light chain comprising 3 CDRs of sequences SEQ ID NOs: 1432, 1633, and 1499, as defined by Kabat.

12. The method of claim 10, wherein the anti-VISTA antibody is a humanized antibody.

13. The method of claim 1, further comprising a step of adapting the treatment with the anti-VISTA therapeutic agent, wherein the adaptation of treatment is:
a reduction or suppression of the anti-VISTA therapeutic agent treatment if the patient has been diagnosed as non-responding to the anti-VISTA therapeutic agent, or
the continuation of the anti-VISTA therapeutic agent treatment if the patient has been diagnosed as responding to the anti-VISTA therapeutic agent.

14.-26. (canceled)

27. The method of claim 8, wherein the hematological cancer is a leukemia, a lymphoma, or a myeloma.
